(19) United States
(12) Patent Application Publication
(10) Pub. No.: US 2026/0265282 A1
(43) Pub. Date: Sep. 10, 2026

(54) SUBSTITUTED 2,3-DIHYDROBENZO[D][1,3]OXAPHOSPHOLE LIGAND, PREPARATION METHOD AND USE THEREOF

(71) Applicants: SHENZHEN CATALYS TECHNOLOGY CO., LTD, Shenzhen (CN); Shenzhen Greencat Pharmaceutical Technology Co., Ltd, Shenzhen (CN)

(72) Inventors: Genqiang CHEN, Shenzhen (CN); Longsheng ZHENG, Shenzhen (CN); Shuo Li, Shenzhen (CN); Jianchao YU, Shenzhen (CN); Bin Lu, Shenzhen (CN)

(73) Assignees: SHENZHEN CATALYS TECHNOLOGY CO., LTD, Shenzhen (CN); Shenzhen Greencat Pharmaceutical Technology Co., Ltd, Shenzhen (CN)

(21) Appl. No.: 19/660,298

(22) Filed: Apr. 28, 2026

Related U.S. Application Data
(63) Continuation of application No. PCT/CN2024/132312, filed on Nov. 15, 2024.

(30) Foreign Application Priority Data
Nov. 17, 2023 (CN) 2023115448005
May 15, 2024 (CN) 2024106081222

(51) Int. Cl. C07F 9/6571; B01J 31/28; C07F 15/00; C07F 17/02

(52) U.S. Cl. CPC C07F 9/657163; B01J 31/28; C07F 15/0066; C07F 17/02

(57) ABSTRACT

The present invention relates to the technical field of catalysts, in particular to a 2,3-dihydrobenzo[d][1,3]oxaphosphole derivative and a preparation method thereof; compound I was constructed in the present invention utilizing tandem nucleophilic addition (or nucleophilic substitution) and intramolecular S_{N}Ar reaction, the method has the characteristics of simple operation steps, high yield, and mild reaction conditions, the technical problems of complex and harsh conditions in the synthesis of oxaphosphinine compounds in a conventional prior art were solved.

### CROSS-REFERENCE TO RELATED APPLICATIONS

This application claims the benefit of Chinese patent applications CN202311544800.5, filed on Nov. 17, 2023, and CN202410608122.2, filed on May 15, 2024, which are incorporated herein by reference in its entirety as a part of this application.

### TECHNICAL FIELD

The invention relates to the technical field of catalysts, to substituted 2,3-dihydrobenzo[d][1,3]oxaphosphole ligand, preparation method and use thereof.

### BACKGROUND

The frameworks of substituted 2,3-dihydrobenzo[d][1,3]oxaphosphole compounds and 3,4-dihydrobenzo[d][1,4]oxaphosphole compounds have electron rich aromatic structures, making them a class of dominant organic phosphine ligand frameworks. The rigid and electron rich phosphine containing cyclic structure can provide a robust coordination environment in catalytic processes, enabling more efficient and specific enantioselective conversion of substrates to products. In fact, such electron rich organic phosphine ligands have been widely developed and applied in cross coupling reactions catalyzed by transition metals, asymmetric hydrogenation reactions catalyzed by transition metals, and asymmetric substitution reactions catalyzed by phosphine catalysts. For example, Tang's research group has developed a series of chiral oxaphosphorene ligands that can achieve cross coupling reactions between aryl bromide with high steric hindrance and boric acid. In addition, they further utilized this type of electron rich chiral ligand to achieve enantioselective cyclization of N-acetylketones. These electron rich phosphine compounds have attracted widespread attention from scientists in recent years due to their excellent coordination activity. However, there are few reports about synthesis of rigid phosphine containing cyclic structural fragments at present. The preparation routes of most electron rich chiral phosphine ligands are quite long, and the preparation process of some chiral ligands also requires complex protection and deprotection processes. Therefore, the development of short and efficient strategies for synthesizing rigid and electron rich phosphine compounds is the research objective of many organic synthesis works. In prior experimental techniques, 2,3-dihydrobenzo[d][1,3]oxaphosphole compounds is obtained mainly through nucleophilic substitution of phosphine α-carbon by oxygen nucleophiles on the aromatic ring. Currently, there is no direct synthetic method for such important benzo[d][1,3]oxaphosphole compounds through tandem nucleophilic addition/aromatic nucleophilic substitution.

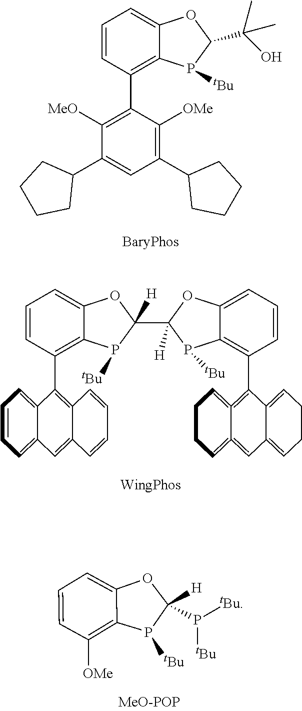

### SUMMARY

In view of the problems that need to be improved in the prior art, the present invention solves the problems by providing a 2,3-dihydrobenzo[d][1,3]oxaphosphole ligand, which exhibits excellent activity in the coupling reaction of C—C bonds between aryl halides and aryl boronic acids or C—N bonds between aromatic amines and palladium complexes with high steric hindrance.

To achieve the above objectives, the present invention provides the follows:

In one aspect, provided herein is a substituted 2,3-dihydrobenzo[d][1,3]oxaphosphole ligand, it is represented by the structure of formula (I),

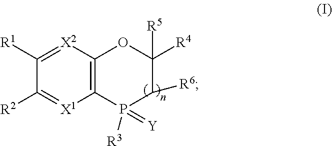

wherein R⁴ is hydrogen, alkyl, alkenyl, cycloalkyl, heterocyclyl, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)—, dicycloalkyloxyphospho(═O)—, ferrocene, aryl, heteroaryl, alkyloxy, cycloalkyloxy, heterocyclyloxy, aryloxy, heteroaryloxy, cycloalkylalkyl, heterocyclylalkyl, arylalkyl or heteroarylalkyl; alternatively, R⁴ and R⁵, together with the carbon atom to which they are attached, form a cycloalkyl or heterocyclyl group; alternatively, R⁴ and the adjacent R⁶, together with the carbon atoms to which they are attached, form a cycloalkyl or heterocyclyl group;
X¹ is N or CR^{X1};
X² is N or CR^{X2}:
each of R^{X1} and R^{X2} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy, SO₂NMe₂ or halo;

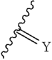

is ═O or ═S; or

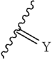

is absent;
R¹ is hydrogen, alkyl, aryl, heteroaryl, alkyloxy, —SO₂NMe₂ or halo;
R² is hydrogen, alkyl, aryl, heteroaryl, alkyloxy, —SO₂NMe₂ or halo;
or R¹ and R², together with the carbon atoms to which they are attached, form an aryl or heteroaryl group;
R³ is hydrogen, alkyl, cycloalkyl, aryl, heteroaryl or alkyloxy;
R⁵ is hydrogen, alkyl, aryl, heteroaryl or alkyloxy;
R⁶ is hydrogen;
n is 0 or 1;
or R⁴ is

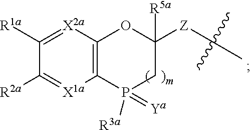

wherein X^{1a} is N or CRX^{1a}, X^{2a} is N or CR^{X2a};
each of R^{X1a} and R^{X2a} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy, fluoro, chloro, bromo or iodo;

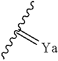

is ═O or ═S; or

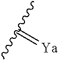

is absent;
Z is a bond, cycloalkylene, heterocyclylene, arylene, heteroarylene, alkylene or

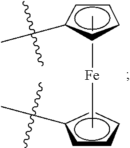

R^{1a} is hydrogen, alkyl, aryl, heteroaryl, alkyloxy or halo;
R^{2a} is hydrogen, alkyl, aryl, heteroaryl, alkyloxy or halo;
or R^{1a} and R^{2a}, together with the carbon atoms to which they are attached, they form an aryl or heteroaryl group;
R^{3a} is hydrogen, alkyl, cycloalkyl, aryl, heteroaryl or alkyloxy;
R^{5a} is hydrogen, alkyl, aryl, heteroaryl or alkyloxy;
m is 0 or 1;
the alkyl, alkenyl, cycloalkyl, heterocyclyl, aryl, heteroaryl, cycloalkylene, heterocyclylene, arylene, heteroarylene and alkylene mentioned in the single groups or complex groups hereinabove are each independently optionally substituted with 1, 2, 3, 4 or 5 substituents selected from hydroxy, amino, halo, carboxyl, sulfonyl, alkyl, haloalkyl, cycloalkyl, heterocyclyl, aryl, heteroaryl, alkyloxy, alkylthio, cycloalkyloxy, heterocyclyloxy, aryloxy, heteroaryloxy, alkyl-NH—, (alkyl)₂N—, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)— and dicycloalkyloxyphospho(═O)—.

In some embodiments, the substituted 2,3-dihydrobenzo[d][1,3]oxaphosphole ligand is represented by the structure of formula I-A or II-A:

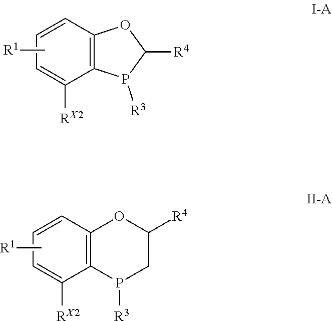

wherein
R¹ is H, F or SO₂NMe₂;
R^{X2} is F, Cl, Br or I;
R³ is aryl or alkyl;
R⁴ is hydrogen, aryl, heteroaryl or alkyl;
the aryl or heteroaryl is unsubstituted or substituted with 1, 2, 3, 4 or 5 substituents selected from halo, PPh₂, —N(alkyl)₂, C₁₋₆alkyl, C₁₋₆haloalkyl, C₁₋₆alkyloxy and C₁₋₆alkylthio.

In some embodiments,
R¹ is H or F;
R^{X2} is F;
R³ is C₆₋₁₀aryl or C₁₋₆alkyl;
R⁴ is C₆₋₁₀aryl, 3-6 membered heteroaryl or C₁₋₆alkyl;
the C₆₋₁₀aryl, 3-6 membered heteroaryl or C₁₋₆alkyl is unsubstituted or substituted with 1, 2, 3, 4 or 5 substituents selected from halo, —PPh₂, —NMe₂, SMe₂, —SMe, C₁₋₆haloalkyl and C₁₋₆alkyloxy.

In some embodiments, the substituted 2,3-dihydrobenzo[d][1,3]oxaphosphole ligand is selected from one of the following structures:

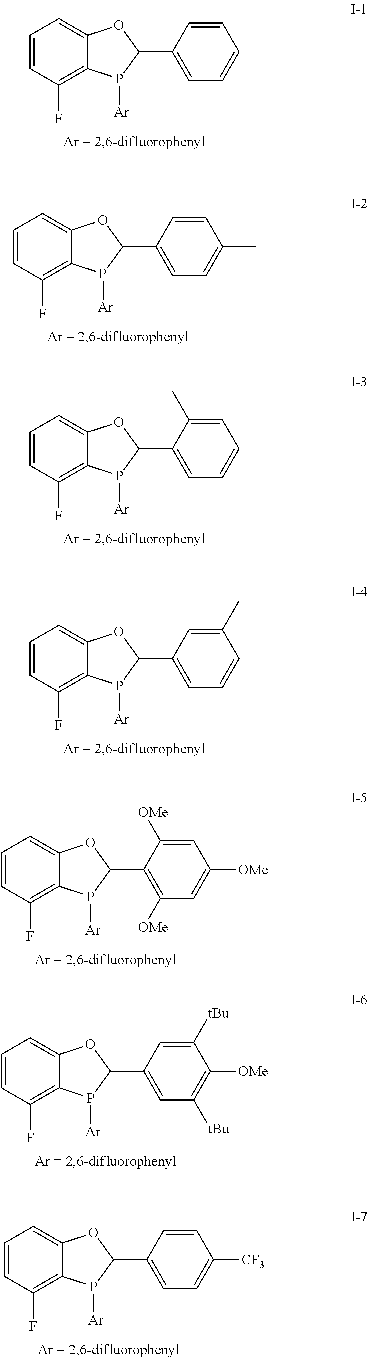

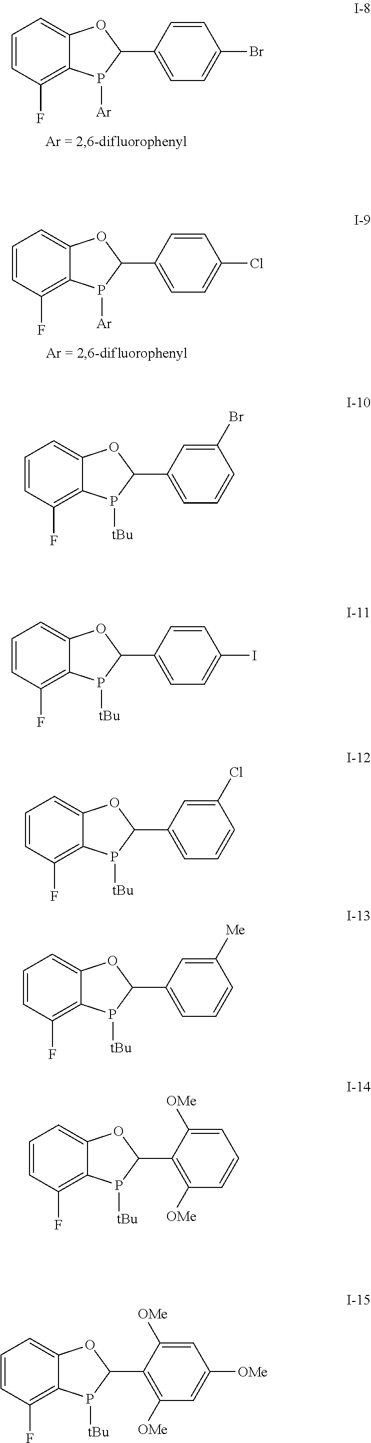

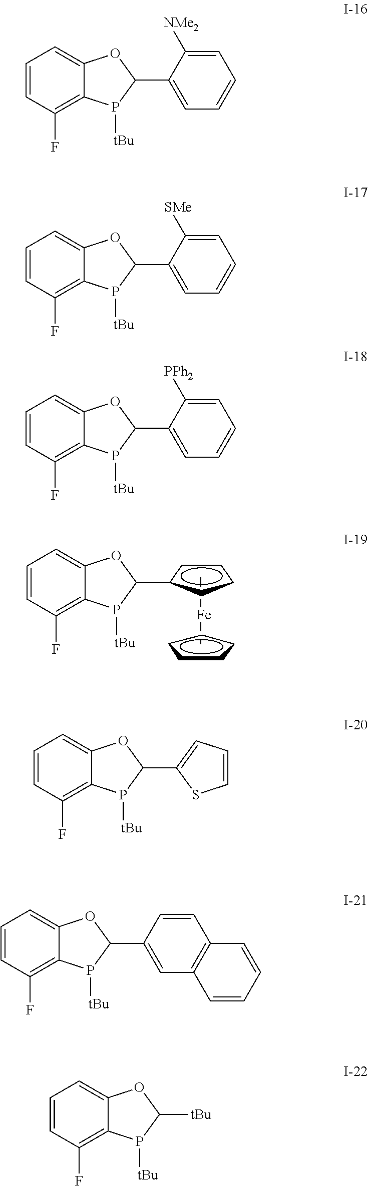

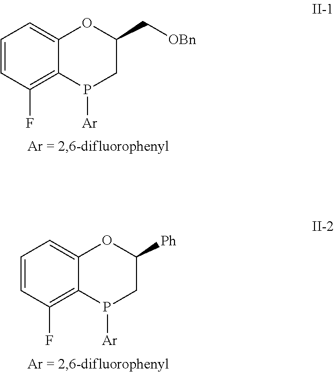

In some embodiments, provided herein is a substituted 2,3-dihydrobenzo[d][1,3]oxaphosphole ligand, it is represented by the structure of formula (I-B),

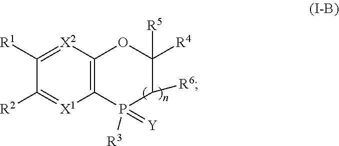

wherein R⁴ is hydrogen, alkyl, alkenyl, cycloalkyl, heterocyclyl, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)—, dicycloalkyloxyphospho(═O)—, ferrocene, aryl, heteroaryl, alkyloxy, cycloalkyloxy, heterocyclyloxy, aryloxy, heteroaryloxy, cycloalkylalkyl, heterocyclylalkyl, arylalkyl or heteroarylalkyl; alternatively, R⁴ and R⁵, together with the carbon atom to which they are attached, form a cycloalkyl or heterocyclyl group; alternatively, R⁴ and the adjacent R⁶, together with the carbon atoms to which they are attached, form a cycloalkyl or heterocyclyl group;
X¹ is N or CR^{X1};
X² is N or CR^{X2};
each of R^{X1} and R^{X2} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy or halo;

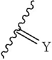

is ═O or ═S; or

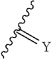

is absent;
R¹ is hydrogen, alkyl, aryl, heteroaryl, alkyloxy or halo;
R² is hydrogen, alkyl, aryl, heteroaryl, alkyloxy or halo;
or R¹ and R², together with the carbon atoms to which they are attached, form an aryl or heteroaryl group;
R³ is hydrogen, alkyl, cycloalkyl, aryl, heteroaryl or alkyloxy;
R⁵ is hydrogen, alkyl, aryl, heteroaryl or alkyloxy;
R⁶ is hydrogen;
n is 0 or 1;
or R⁴ is

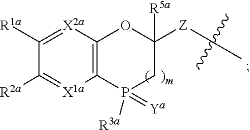

wherein X^{1a} is N or CRX^{1a}, X^{2a} is N or CR^{X2a};
each of R^{X1a} and R^{X2a} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy, fluoro, chloro, bromo or iodo;

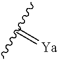

is ═O or ═S; or

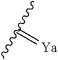

is absent;
Z is a bond, cycloalkylene, heterocyclylene, arylene, heteroarylene, alkylene or

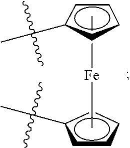

R^{1a} is hydrogen, alkyl, aryl, heteroaryl, alkyloxy or halo;
R^{2a} is hydrogen, alkyl, aryl, heteroaryl, alkyloxy or halo;
or R^{1a} and R^{2a}, together with the carbon atoms to which they are attached, form an aryl or heteroaryl group;
R^{3a} is hydrogen, alkyl, cycloalkyl, aryl, heteroaryl or alkyloxy;
R^{5a} is hydrogen, alkyl, aryl, heteroaryl or alkyloxy;
m is 0 or 1;
the alkyl, alkenyl, cycloalkyl, heterocyclyl, aryl, heteroaryl, cycloalkylene, heterocyclylene, arylene, heteroarylene and alkylene mentioned in the single groups or complex groups hereinabove are each independently optionally substituted with 1, 2, 3, 4 or 5 substituents selected from hydroxy, amino, halo, carboxyl, sulfonyl, alkyl, haloalkyl, cycloalkyl, heterocyclyl, aryl, heteroaryl, alkyloxy, alkylthio, cycloalkyloxy, heterocyclyloxy, aryloxy, heteroaryloxy, alkyl-NH—, (alkyl)₂N—, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)— and dicycloalkyloxyphospho(═O)—;
with the proviso that the structures of formula (I-B) do not include the following structures:

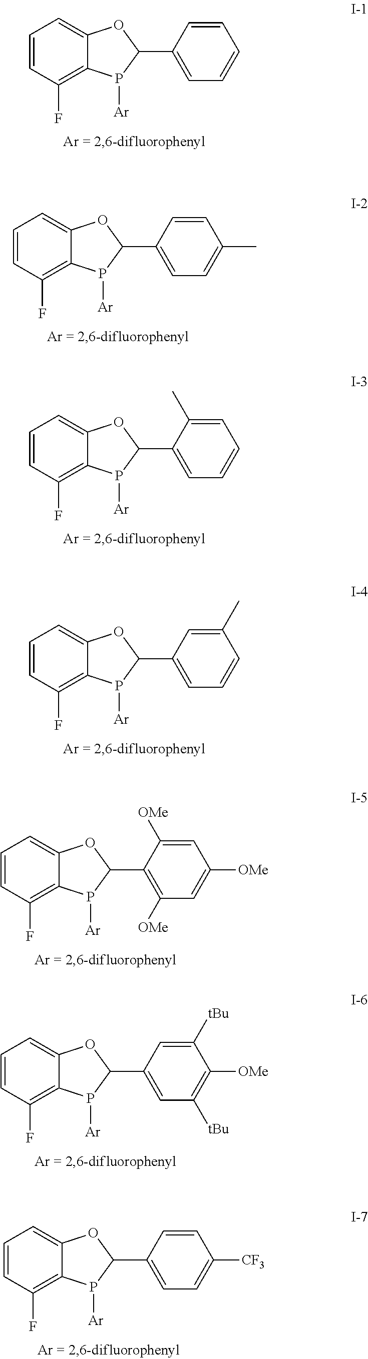

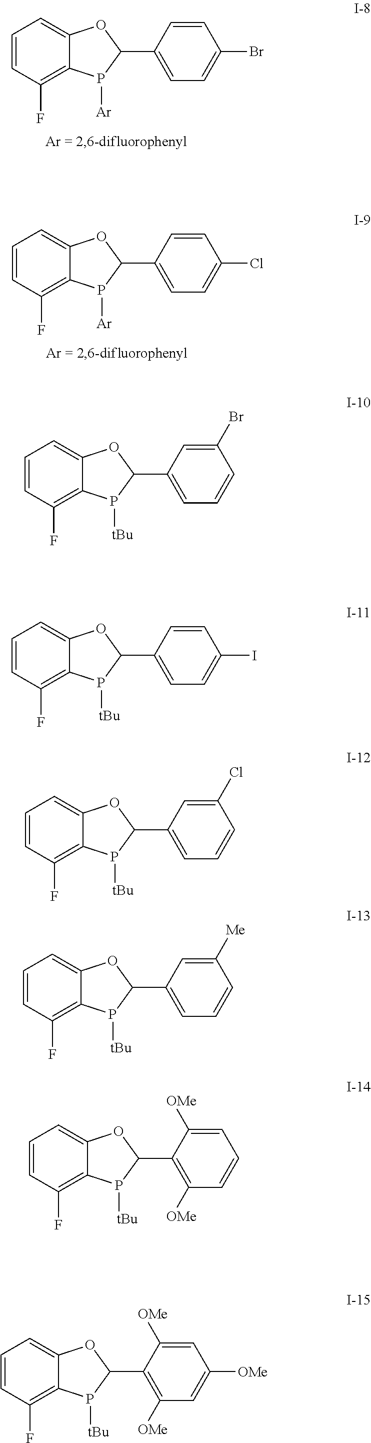

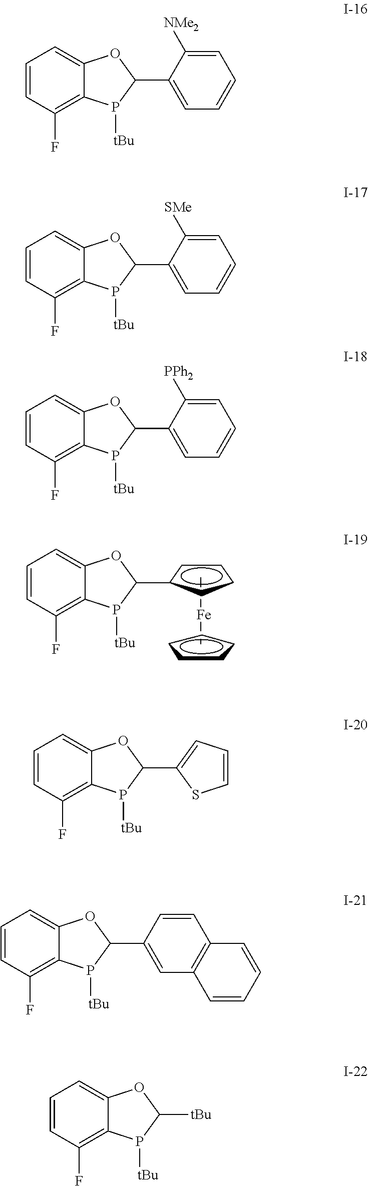

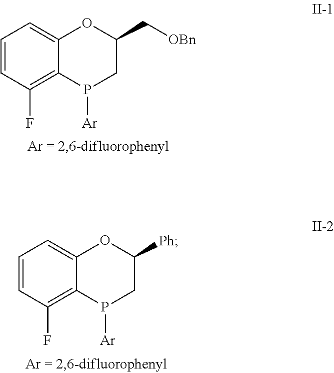

or with the proviso that in the structure of formula (I-B), when R^{X1a} is F,

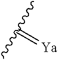

is ═O or ═S;
or with the proviso that in the structure of formula (I-B), when R^{X1a} is F, R⁴ is hydrogen, alkyl, alkenyl, cycloalkyl, heterocyclyl, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)—, dicycloalkyloxyphospho(═O)—, ferrocene, alkyloxy, cycloalkyloxy, heterocyclyloxy, aryloxy, heteroaryloxy, cycloalkylalkyl, heterocyclylalkyl, arylalkyl or heteroarylalkyl; alternatively, R⁴ and R⁵, together with the carbon atom to which they are attached, form a cycloalkyl or heterocyclyl group; alternatively, R⁴ and the adjacent R⁶, together with the carbon atoms to which they are attached, form a cycloalkyl or heterocyclyl group; or R⁴ is

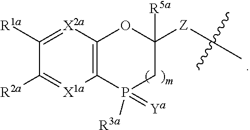

In some embodiments, each of

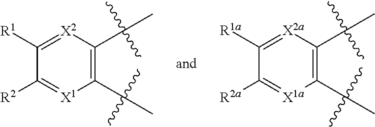

is independently selected from:

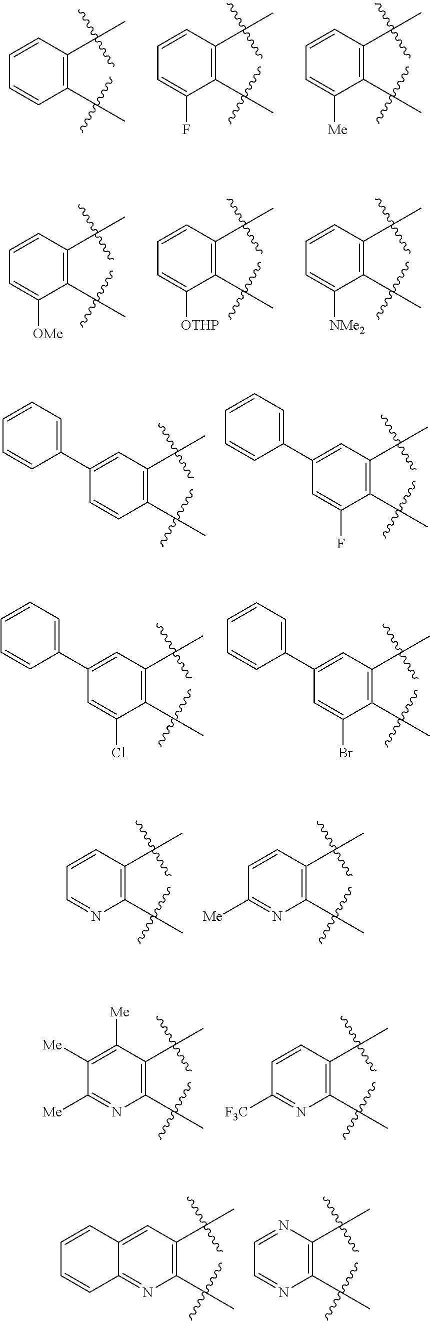

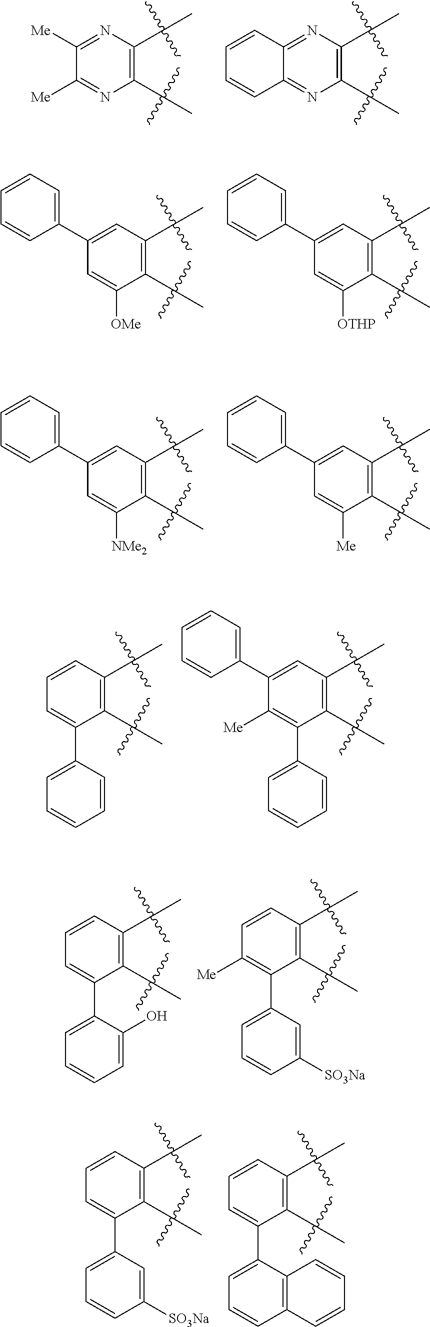

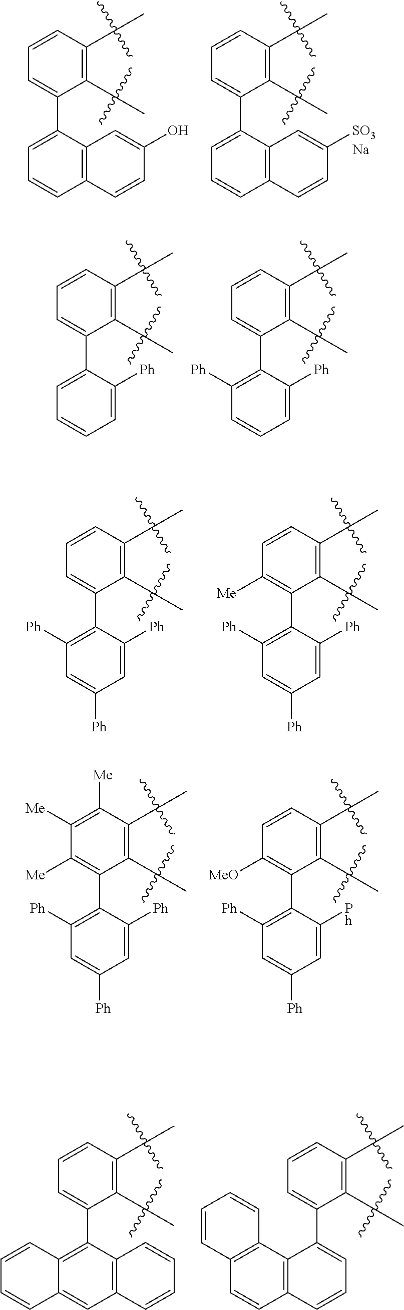

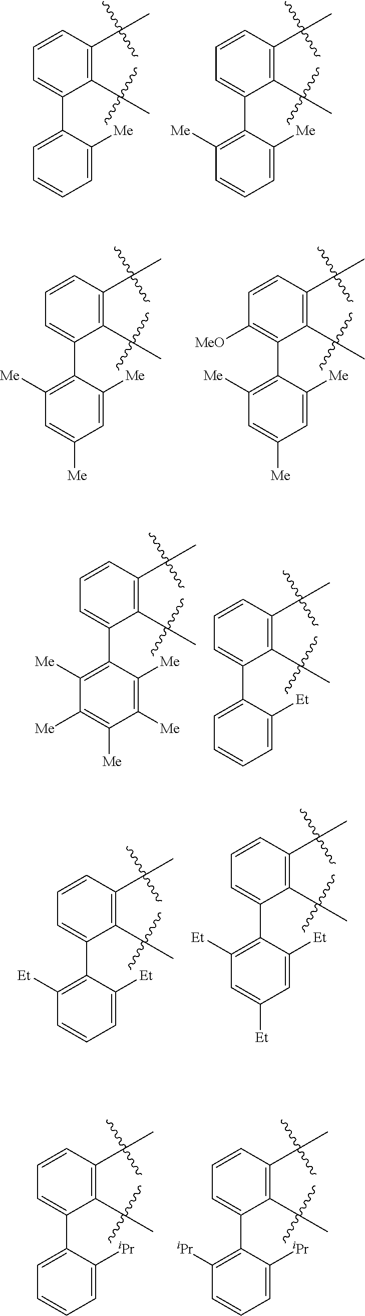

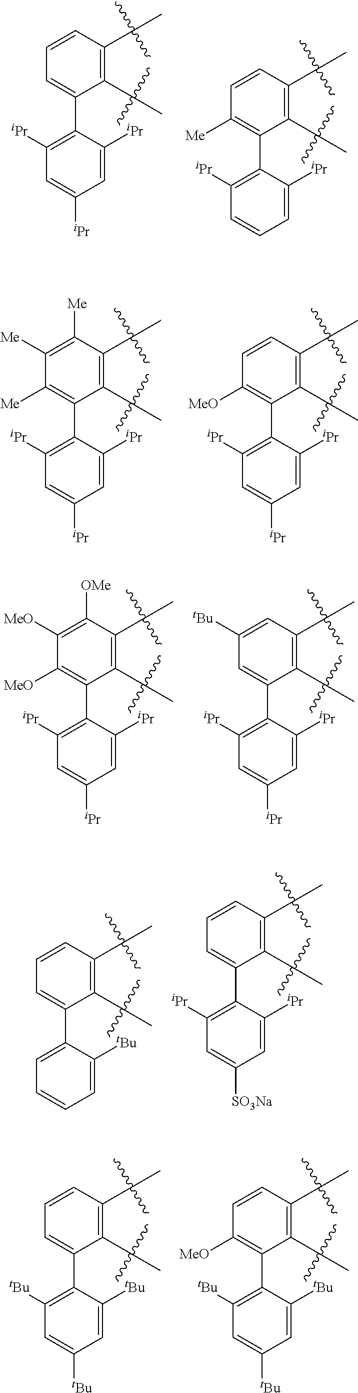

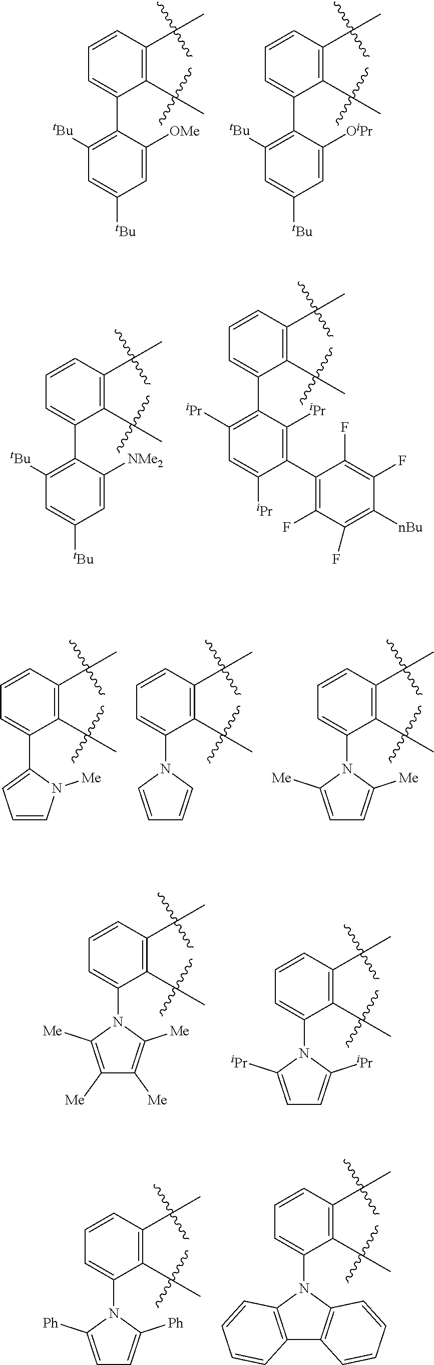

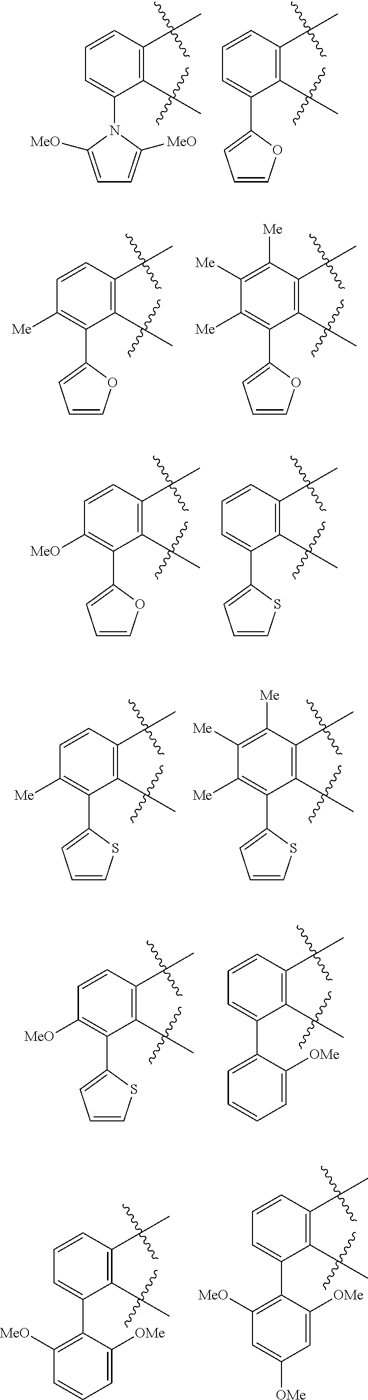

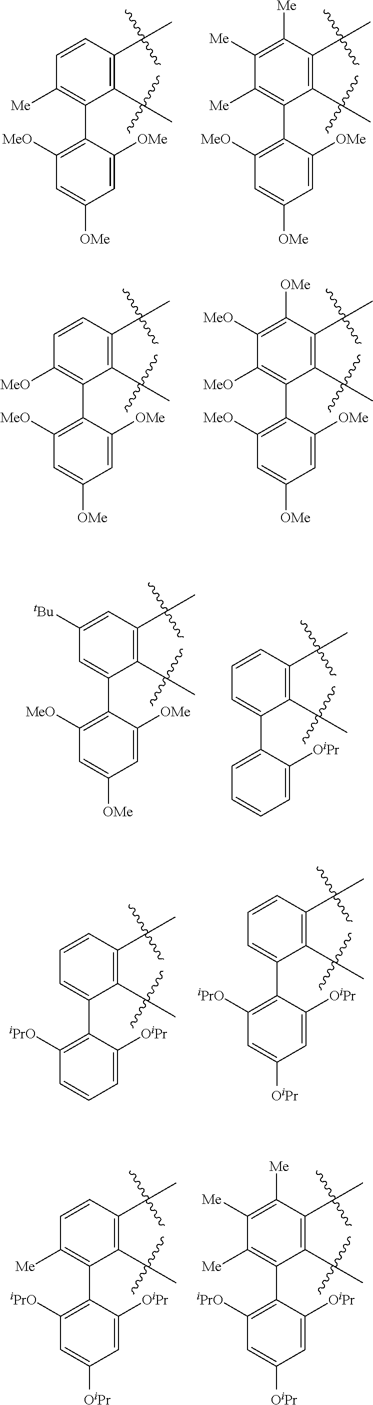

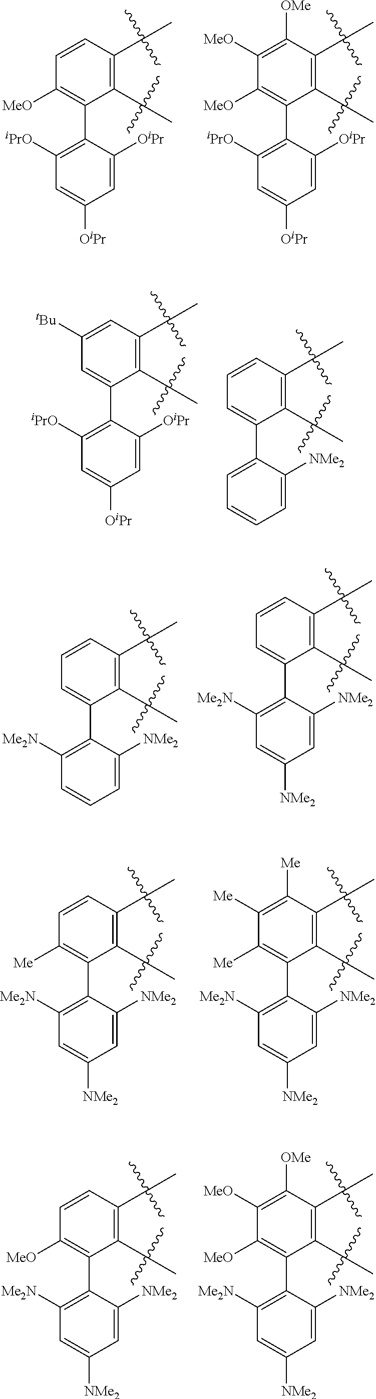

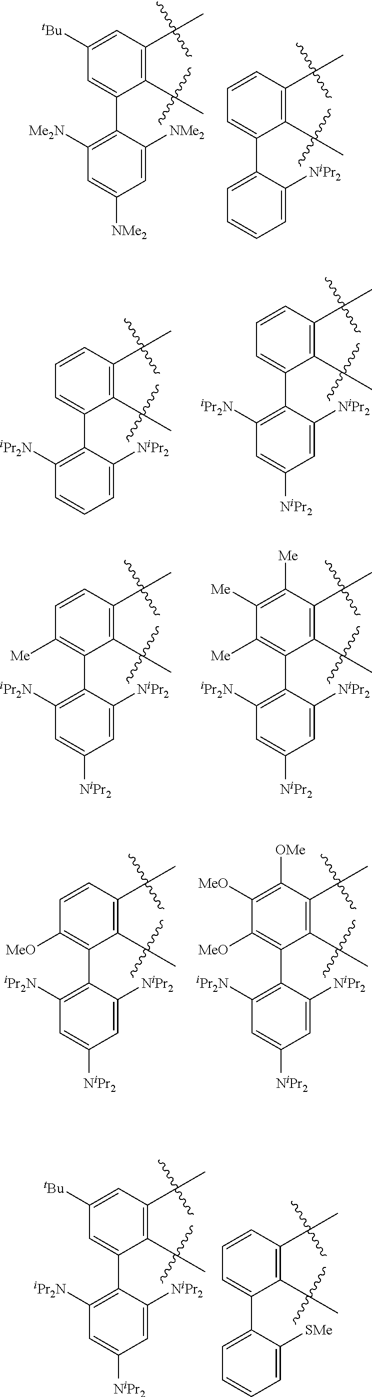

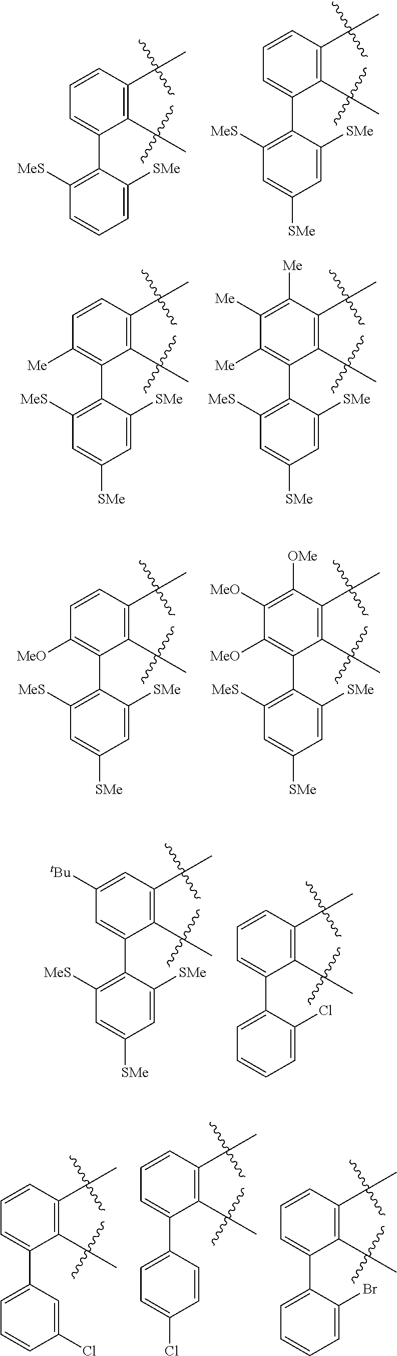

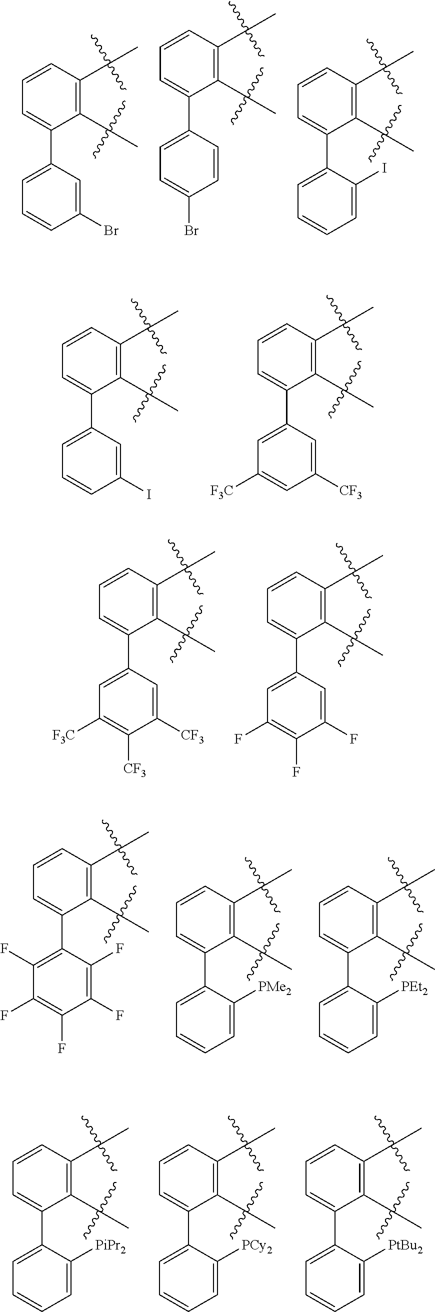

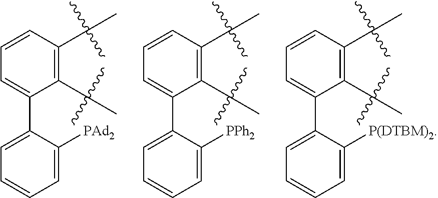

In some embodiments, R⁴ is selected from:

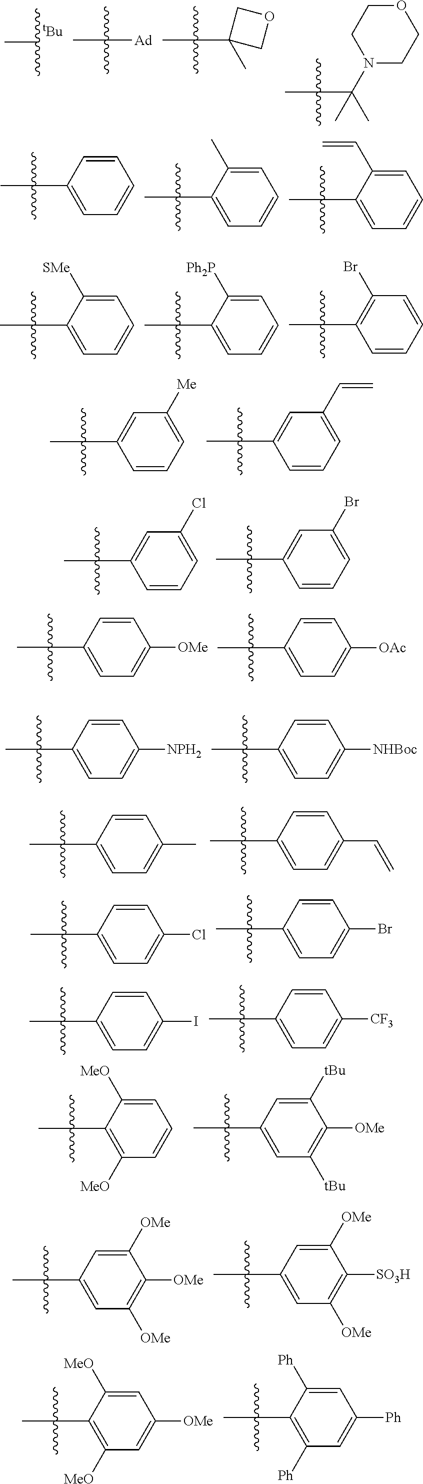

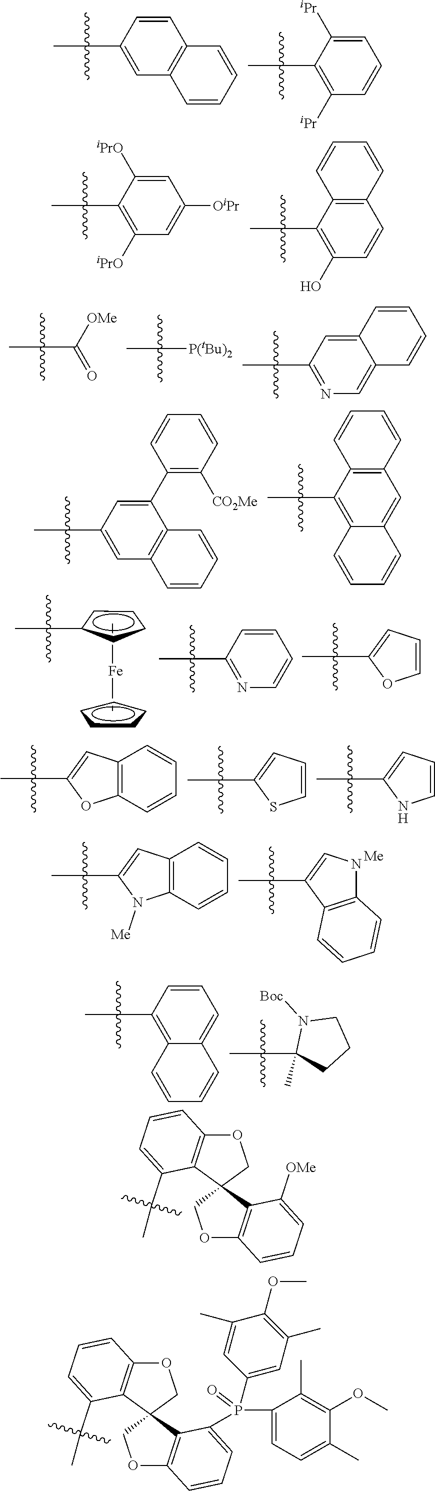

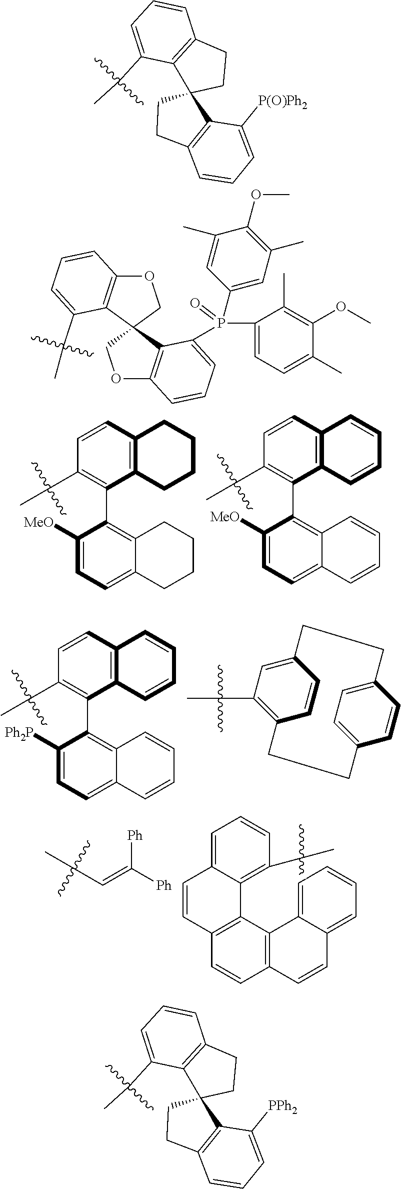

In some embodiments, each of R³ and R^{3a} is independently selected from:

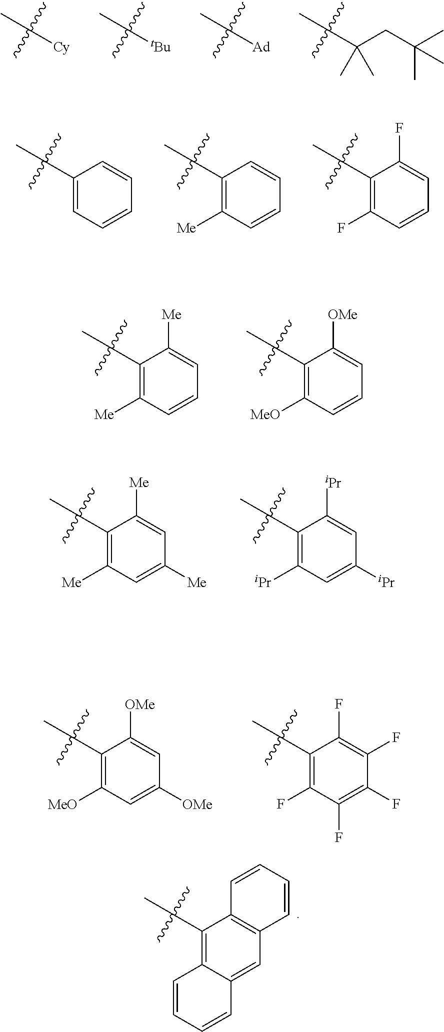

In some embodiments, Z is selected from:

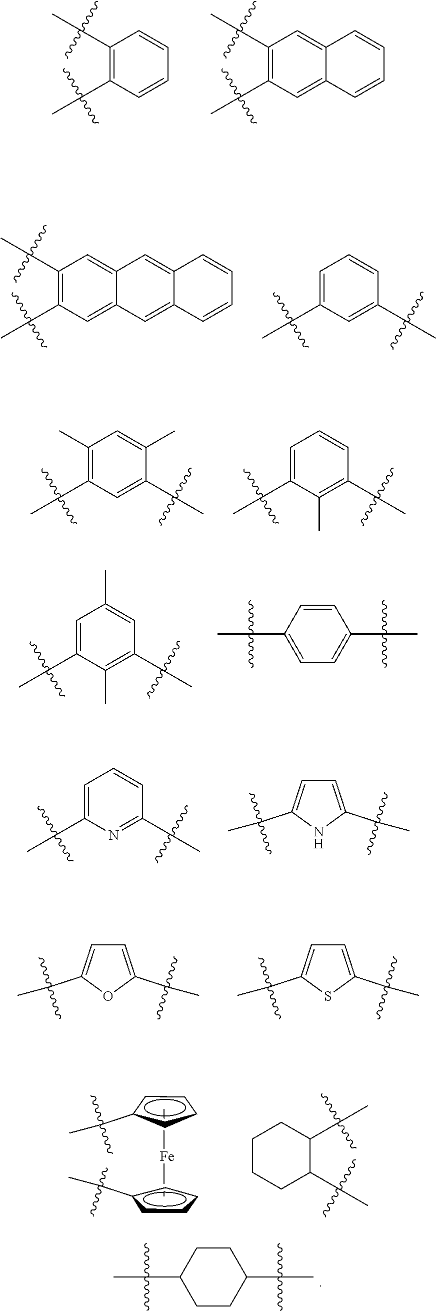

In some embodiments, the compound of formula I-B is selected from:

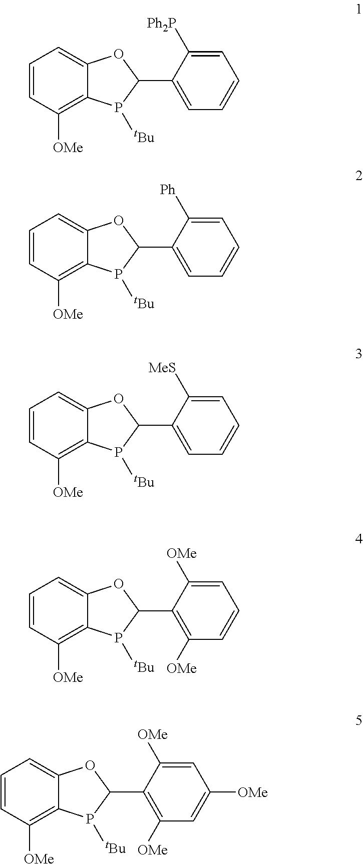

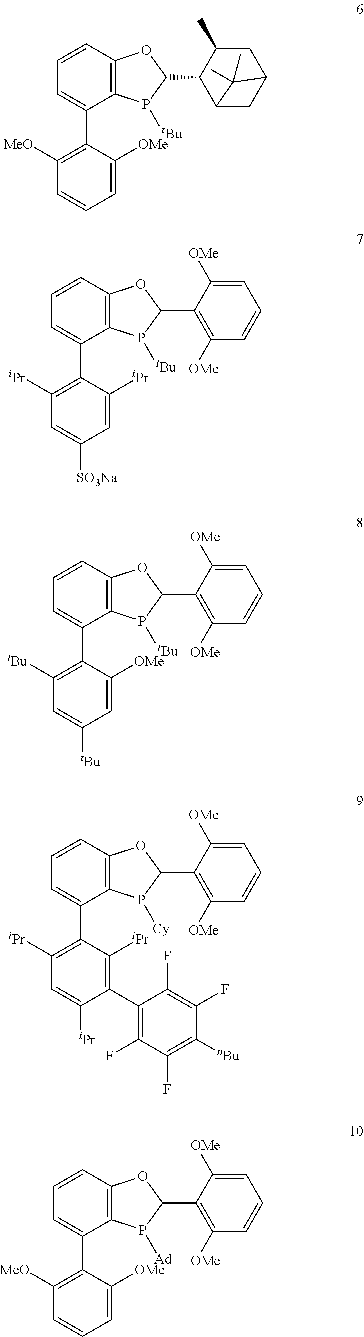

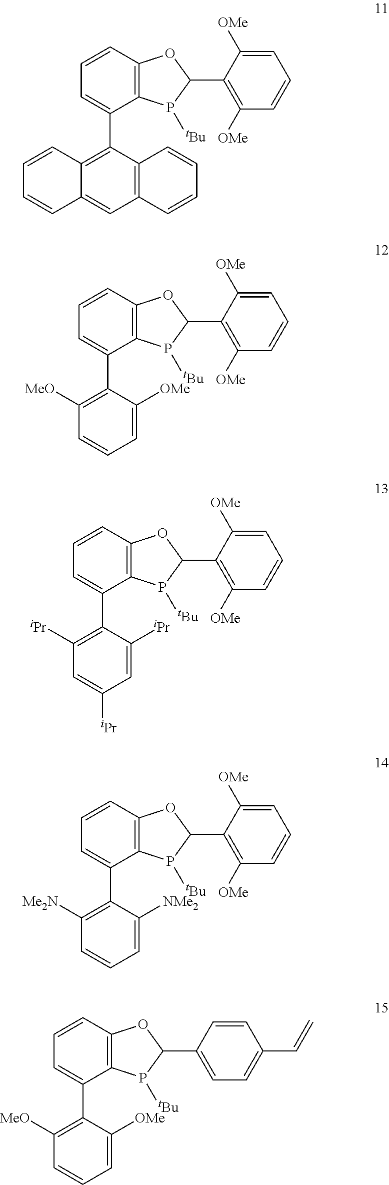 [SMILES: COc1cccc(OC)c1C1Oc2cccc(-c3c4ccccc4cc4ccccc34)c2P1C(C)(C)C ; COc1cccc(OC)c1-c1cccc2c1P(C(C)(C)C)C(c1c(OC)cccc1OC)O2 ; COc1cccc(OC)c1C1Oc2cccc(-c3c(C(C)C)cc(C(C)C)cc3C(C)C)c2P1C(C)(C)C ; COc1cccc(OC)c1C1Oc2cccc(-c3c(N(C)C)cccc3N(C)C)c2P1C(C)(C)C ; C=Cc1ccc(C2Oc3cccc(-c4c(OC)cccc4OC)c3P2C(C)(C)C)cc1]

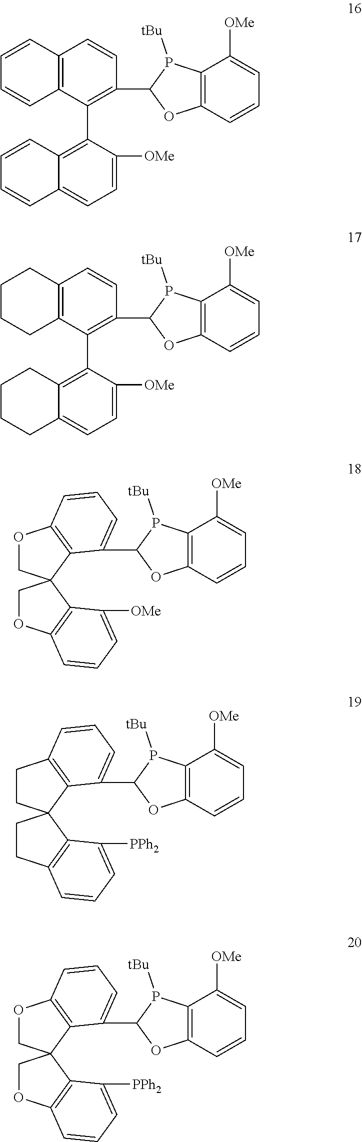

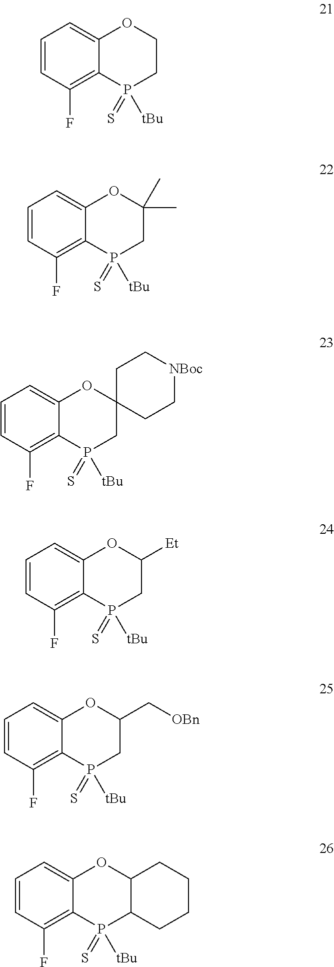 [SMILES: CC(C)(C)P1(=S)CCOc2cccc(F)c21 ; CC1(C)CP(=S)(C(C)(C)C)c2c(F)cccc2O1 ; CC(C)(C)OC(=O)N1CCC2(CC1)CP(=S)(C(C)(C)C)c1c(F)cccc1O2 ; CCC1CP(=S)(C(C)(C)C)c2c(F)cccc2O1 ; CC(C)(C)P1(=S)CC(COCc2ccccc2)Oc2cccc(F)c21 ; CC(C)(C)P1(=S)c2c(F)cccc2OC2CCCCC21]

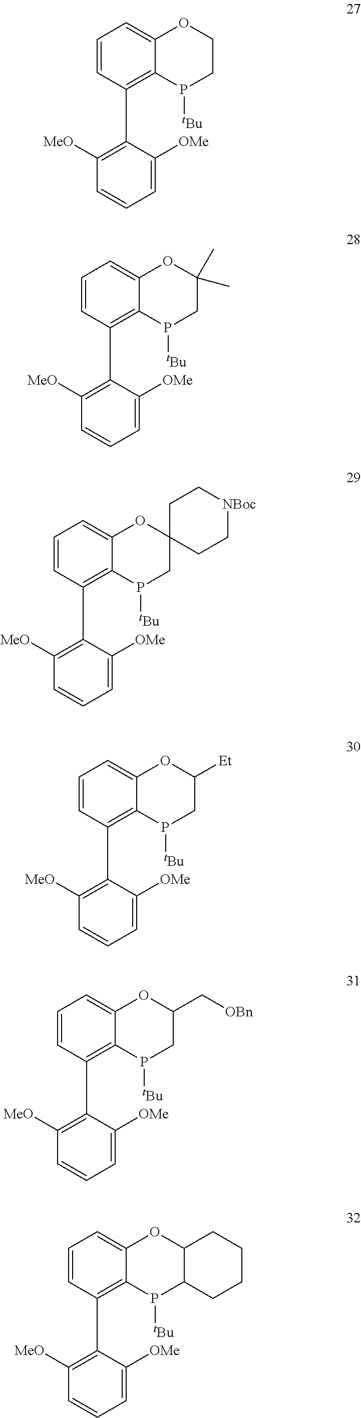 [SMILES: COc1cccc(OC)c1-c1cccc2c1P(C(C)(C)C)CCO2 ; COc1cccc(OC)c1-c1cccc2c1P(C(C)(C)C)CC(C)(C)O2 ; COc1cccc(OC)c1-c1cccc2c1P(C(C)(C)C)CC1(CCN(C(=O)OC(C)(C)C)CC1)O2 ; CCC1CP(C(C)(C)C)c2c(cccc2-c2c(OC)cccc2OC)O1 ; COc1cccc(OC)c1-c1cccc2c1P(C(C)(C)C)CC(COCc1ccccc1)O2 ; COc1cccc(OC)c1-c1cccc2c1P(C(C)(C)C)C1CCCCC1O2]

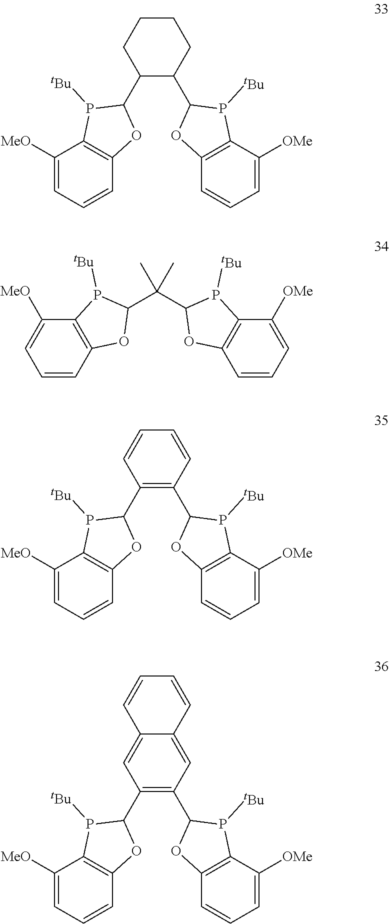 [SMILES: COc1cccc2c1P(C(C)(C)C)C(C1CCCCC1C1Oc3cccc(OC)c3P1C(C)(C)C)O2 ; COc1cccc2c1P(C(C)(C)C)C(C(C)(C)C1Oc3cccc(OC)c3P1C(C)(C)C)O2 ; COc1cccc2c1P(C(C)(C)C)C(c1ccccc1C1Oc3cccc(OC)c3P1C(C)(C)C)O2 ; COc1cccc2c1P(C(C)(C)C)C(c1cc3ccccc3cc1C1Oc3cccc(OC)c3P1C(C)(C)C)O2]

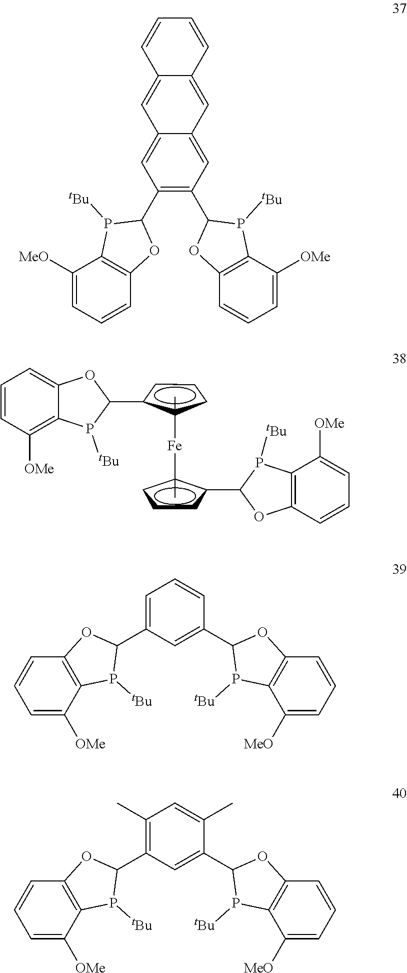

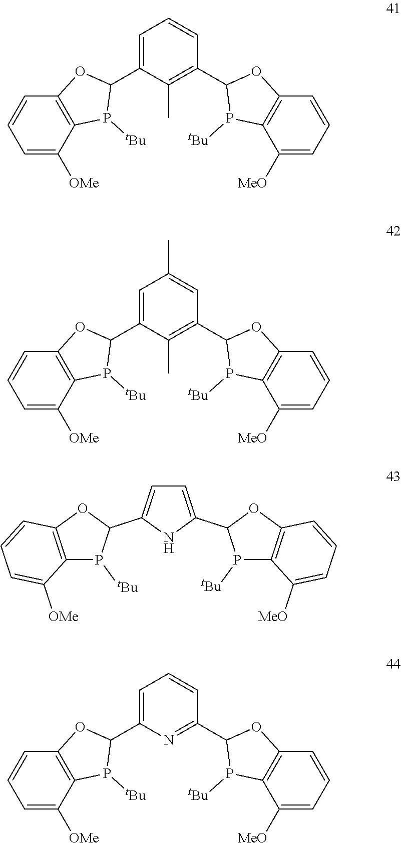 [SMILES: COc1cccc2c1P(C(C)(C)C)C(c1cccc(C3Oc4cccc(OC)c4P3C(C)(C)C)c1C)O2 ; COc1cccc2c1P(C(C)(C)C)C(c1cc(C)cc(C3Oc4cccc(OC)c4P3C(C)(C)C)c1C)O2 ; COc1cccc2c1P(C(C)(C)C)C(c1ccc(C3Oc4cccc(OC)c4P3C(C)(C)C)[nH]1)O2 ; COc1cccc2c1P(C(C)(C)C)C(c1cccc(C3Oc4cccc(OC)c4P3C(C)(C)C)n1)O2]

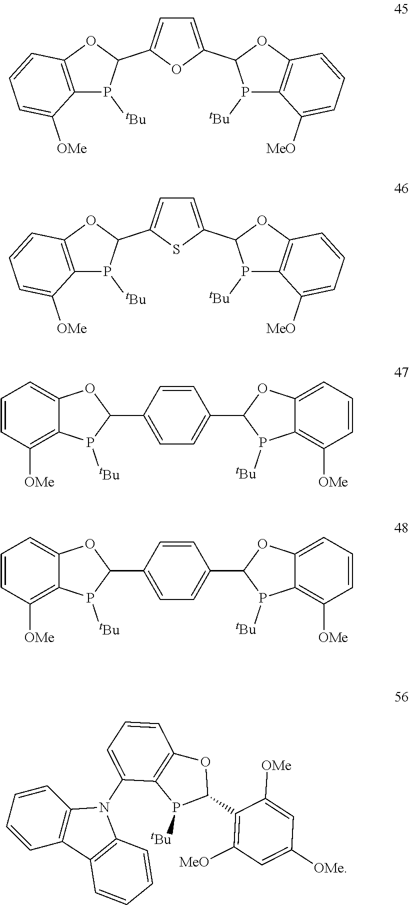 [SMILES: COc1cccc2c1P(C(C)(C)C)C(c1ccc(C3Oc4cccc(OC)c4P3C(C)(C)C)o1)O2 ; COc1cccc2c1P(C(C)(C)C)C(c1ccc(C3Oc4cccc(OC)c4P3C(C)(C)C)s1)O2 ; COc1cccc2c1P(C(C)(C)C)C(c1ccc(C3Oc4cccc(OC)c4P3C(C)(C)C)cc1)O2 ; COc1cccc2c1P(C(C)(C)C)C(c1ccc(C3Oc4cccc(OC)c4P3C(C)(C)C)cc1)O2 ; COc1cc(OC)c([C@H]2Oc3cccc(-n4c5ccccc5c5ccccc54)c3[P@]2C(C)(C)C)c(OC)c1]

Me: methyl; ^{t}Bu: tert butyl; Et: ethyl; Cy: cyclohexyl; ⁱPr: isopropyl; Ph: phenyl; Ad: adamantly.

In other aspect, provided herein is a diphosphine acetate palladium complex, it is represented by the structure of formula (II-B),

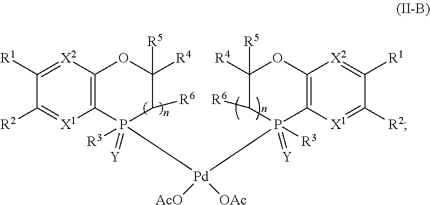

wherein R¹, R², R³, R⁴, R⁵, R⁶, X¹, X³, Y and n have the definition described in the present invention.

In some embodiments, the diphosphine acetate palladium complex has

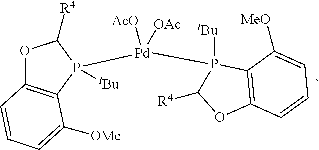

R⁴ has the definition described in the present invention.

In some embodiments, the diphosphine acetate palladium complex has

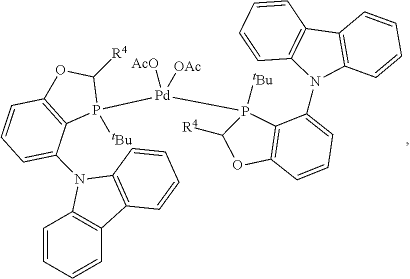

R⁴ has the definition described in the present invention.

In other aspect, provided herein is a coupling reaction method, the method uses a biphosphine acetate palladium complex prepared from a substituted benzophosphine ligand described herein or a biphosphine acetate palladium complex described herein as a catalyst.

In some embodiments, the coupling reaction method is C—C coupling reaction, which comprises reacting aryl halides and aryl boronic acid compounds in an organic solvent under alkaline conditions in the presence of the bisphosphonate palladium acetate complex; or the coupling reaction method is C—N coupling reaction, which comprises reacting aryl halides and aromatic amine compounds in an organic solvent under alkaline conditions in the presence of the bisphosphonate palladium acetate complex;
the solvent is one or more of N,N-dimethylformamide, toluene, benzotrifluoride, carbon tetrachloride, dioxane, hexafluoroisopropanol or ethyl acetate; the base is one or more of potassium tert-butoxide, sodium tert-butoxide, lithium methoxide, cesium carbonate, sodium acetate, sodium hydroxide, potassium hydroxide or sodium hydroxide.

In some embodiments, the aryl boronic acid compounds include aryl boronic acid or aryl borate esters; the aryl halides include aryl chlorides and aryl bromides; the aromatic amine compounds include aromatic primary amine or aromatic secondary amine.

In some embodiments, the use of bisphosphopalladium acetate in the C—C bond formation reaction between aryl halides (such as aryl bromides) and aryl boronic acids:

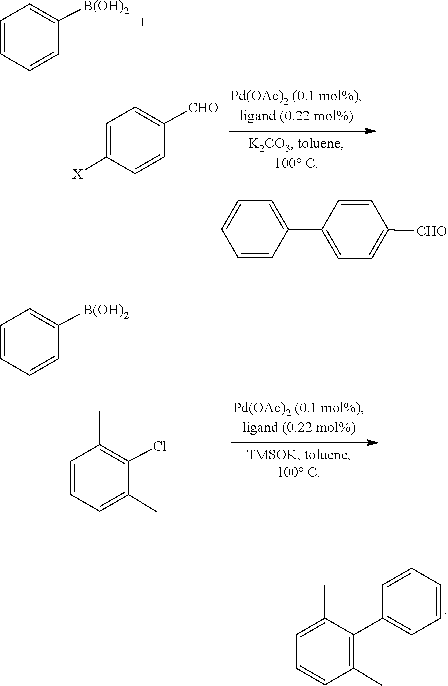

wherein X is chloro or bromo. The used ligand is

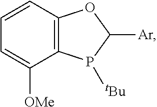

preferable structure is

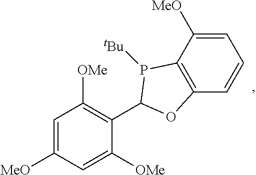 [SMILES: COc1cc(OC)c(C2Oc3cccc(OC)c3P2C(C)(C)C)c(OC)c1]

wherein the amount of catalyst is 0.1%.

In some embodiments, the use of bisphosphopalladium acetate in the C—N bond formation reaction between aryl halides (such as aryl chlorides) and aromatic amine compounds:

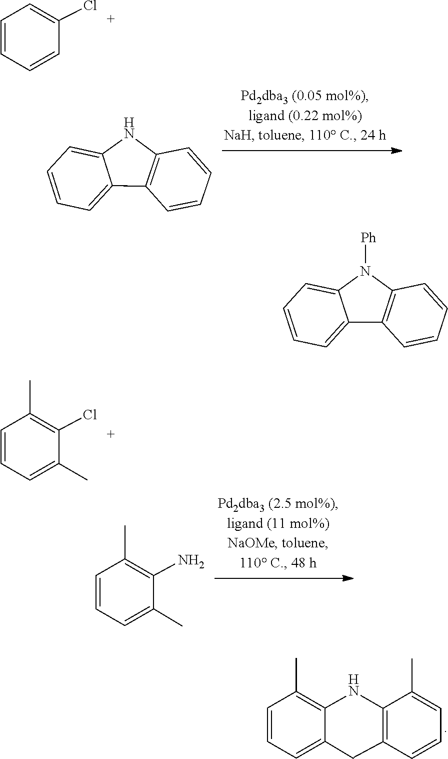

wherein X is chloro or bromo. The used ligand is

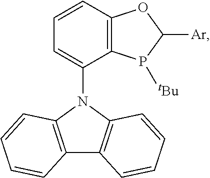

preferable structure is

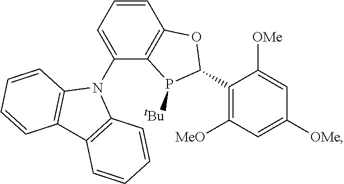 [SMILES: COc1cc(OC)c([C@H]2Oc3cccc(-n4c5ccccc5c5ccccc54)c3[P@]2C(C)(C)C)c(OC)c1]

wherein the amount of catalyst is 0.05-2.5%.

In other aspect, provided herein is a preparation method of a substituted 2,3-dihydrobenzo[d][1,3]oxaphosphole ligand comprising:
S11. stirring formula III with a base in an organic solvent to obtain a salt solution of formula III under inert gas protection,
S12. adding the aldehyde of formula IV or epoxide derivative V to the obtained salt solution of formula III, and reacting at 0-50° C. to obtain the 2,3-dihydrobenzo[d][1,3]oxaphosphole derivative of formula I;

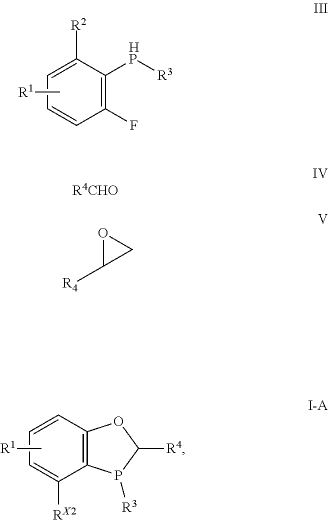

wherein R¹ is H, F or SO₂NMe₂;
R^{X2} is F, Cl, Br or I;
R³ is aryl or alkyl;
R⁴ is aryl, heteroaryl or alkyl;
the aryl or heteroaryl is unsubstituted or substituted with 1, 2, 3, 4 or 5 substituents selected from halo, PPh₂, —NH(alkyl), —N(alkyl)₂, C₁₋₆alkyl, C₁₋₆haloalkyl, C₁₋₆alkyloxy and C₁₋₆alkylthio.

The solvent described in the present invention is not particularly limited, if it can dissolve the reaction raw materials represented by formulas (III), (IV), and (V) to a certain extent and does not inhibit the reaction. In some embodiments, the organic solvent is selected from one or more of 1,4-dioxane, tetrahydrofuran, ether, dichloromethane, 1,2-dichloroethane, toluene, n-hexane, or dimethyl sulfoxide; the base is selected from one or more of cesium carbonate, potassium carbonate, sodium carbonate, potassium tert butoxide, sodium tert butoxide, lithium tert butoxide, sodium ethoxide, sodium acetate or sodium hydroxide.

In some embodiments, the organic solvent is selected from one or more of 1,4-dioxane, tetrahydrofuran, ether, dichloromethane, 1,2-dichloroethane, toluene, n-hexane, or dimethyl sulfoxide.

In some embodiments, the base is selected from one or more of cesium carbonate, potassium carbonate, sodium carbonate, potassium tert butoxide, sodium tert butoxide, lithium tert butoxide, sodium ethoxide, sodium acetate or sodium hydroxide.

In some embodiments, in the mixed system in step S12, the molar ratio of arylphosphine hydrogen of formula III, aldehyde of formula IV of epoxy compound of formula V, and base is 1:1.5:1.5.

In some embodiments, in the mixed system in step S12, the molar ratio of arylphosphine hydrogen of formula III, epoxy compound of formula V, and base is 1:1.5:1.5.

The method described herein may also include suitable reaction conditions for use, some non limiting examples of which include the use of other inert solvents, reagents such as bases, and catalysts. The methods described herein may also include known purification methods such as crystallization, chromatography (liquid chromatography and gas chromatograph), extraction, distillation, development, and reverse phase HPLC. The reaction conditions such as temperature, reaction time, pressure, and gas (such as inert gas, air) may be adjusted appropriately according to the reaction.

The technical solution of the present invention has the following beneficial effects:

The present invention provides a series of 2-halo2,3-dihydrobenzo[d][1,3]oxaphosphole compounds, which have electron rich phosphine atoms. In addition, the product molecules have active halogen groups such as fluorine atoms, which facilitate further functionalization to prepare complex phosphine ligands. More importantly, this strategy is based on tandem nucleophilic addition (nucleophilic substitution)/aromatic nucleophilic substitution reactions, greatly shortening the synthetic route for constructing 2,3-dihydrobenzo[d][1,3]oxaphosphole compounds. The applicant developed a highly efficient tandem nucleophilic addition/aromatic nucleophilic substitution reaction without the involvement of metals, starting from easily obtainable phosphine hydrogen and aldehydes, and efficiently synthesized 2,3-dihydrobenzo[d][1,3]oxaphosphole derivatives with simple operation steps and mild conditions.

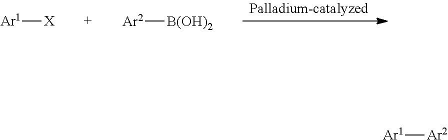

Ar¹, Ar²=aryl or heteroaryl.

### Beneficial Effects

The present invention provides a series of 2,3-dihydrobenzo[d][1,3]oxaphosphole ligands and their applications in C—C and C—N bond coupling of aryl halides. The beneficial effects compared to the prior art include:
(1) The operation steps of such ligands are brief and can start from easily obtainable aldehydes. Secondly, these ligands have electron rich, rigid structures, and air stability.
(2) The present invention can provide a phosphine ligand for palladium catalyzed coupling reactions of aryl halides, and has good reaction results.

Additional aspects and advantages of the present invention will be given in part in the following description, and will become apparent in part from the following description, or will be learned through the practice of the present invention.

### Definitions and Terms

Certain embodiments of the present invention are now described in detail, and examples thereof are illustrated by the accompanying structural Formulas and chemical Formula. The invention is intended to cover all alternatives, modifications, and equivalents, which are included within the scope of the invention as defined by the claims. Those skilled in the art will recognize that many methods and materials similar or equivalent to those described herein can be used to practice the present invention. The present invention is in no way limited to the methods and materials described herein. If one or more of the incorporated literature, patents, and similar materials differs from or contradicts this application (including but not limited to defined terms, terminology applications, described technologies, etc.), it is based on this application.

It will be further appreciated that certain features of the invention have been described in various independent embodiments for clarity of the disclosure, but may also be provided in combination in a single embodiment. Conversely, various features of the invention are described in a single embodiment for the sake of brevity, but may also be provided separately or in any suitable subcombination.

Unless otherwise illustrated, all technical and scientific terms used herein have the same meaning as commonly understood by the person skilled in the art to which this invention belongs. All patents and publications referred to in this application are incorporated herein by reference in their entirety.

Reference throughout this specification to “an embodiment,” “some embodiments,” “an example,” “a specific example,” or “some examples,” means that a particular feature, structure, material, or characteristic described in connection with the embodiment or example is included in at least one embodiment or example of the present disclosure. Thus, the appearances of the above terms throughout this specification are not necessarily referring to the same embodiment or example of the present disclosure. Furthermore, the features, structures, materials, or characteristics may be combined in any suitable manner in one or more embodiments or examples. In addition, those skilled in the art can integrate and combine different embodiments, examples or the features of them if they are not contradictory to one another.

In the following content, regardless of whether the words “approximately” or “about” are used, all numbers disclosed here are approximate values. The value of each number may vary by 1%, 2%, 5%, 7%, 8%, 10%, 15%, or 20%. Whenever a number with a value of N is publicly disclosed, any number with a value of N+/−1%, N+/−2%, N+/−3%, N+/−5%, N+/−7%, N+/−8%, N+/−10%, N+/−15%, or N+/−20% will be explicitly disclosed, where “+/−” refers to addition or subtraction.

A single group refers to a group formed from one type of functional group, such as alkyl, aryl, heteroaromatic, etc; a complex group refers to a group formed by the combination of two or more groups, such as arylalkyl, alkyloxy, alkylthio, cycloalkoxyoxy, heterocyclyloxy, bicycloalkylphospho, etc; The complex groups mentioned herein are uniformly connected to the rest of the molecule by the right side group, such as arylalkyl, the aryl group is connected to the alkyl group, and the complex group is connected to the rest of the molecule with the alkyl group as the connecting point.

In this article, the alkyl group referred to, whether as a single group or as a part of a complex group, represents a saturated linear or branched alkyl group containing carbon atoms. In one embodiment, the alkyl group contains 1-6 carbon atoms, i.e., a C₁₋₆alkyl group; In another embodiment, the alkyl group contains 1~4 carbon atoms, i.e., a C₁₋₄ alkyl group; In another implementation scheme, the alkyl group contains 1-3 carbon atoms, namely a C₁₋₃ alkyl group. Examples of alkyl groups include but are not limited to methyl, ethyl, n-propyl, isopropyl, n-butyl, tert-butyl, isobutyl, sec-butyl, n-pentyl, n-hexyl, and similar alkyl groups.

In this article, the alkenyl group referred to, whether as a single group or as a part of a complex group, represents a linear or branched monovalent hydrocarbon group containing carbon atoms, which has at least one unsaturated site, i.e a carbon carbon sp2 double bond, including the positioning of “cis” and “trans”, or the positioning of “E” and “Z”. In one embodiment, the alkenyl group contains 2-6 carbon atoms, i.e. C₂₋₆ alkenyl; In another implementation, the alkenyl group contains 2-4 carbon atoms, i.e. C₂₋₄ alkenyl. Examples of alkenyl groups include, but are not limited to, vinyl (—CH═CH₂), allyl (—CH₂CH═CH₂), and so on.

In this article, the cycloalkyl group referred to, whether as a single group or as a part of a complex group, represents a monovalent or multivalent monocyclic, bicyclic or tricyclic system containing carbon atoms (e.g. monocyclic, such as cyclopropyl, cyclobutyl, cyclopentyl, cyclohexyl, cycloheptyl, cyclooctyl, cyclononyl, or bicyclic, including spirocyclic, fused or bridged systems (such as bicyclic [1.1.1] pentyl, bicyclic [2.2.1] heptyl, bicyclic [3.2.1] octyl or bicyclic [5.2.0] nonyl, decahydronaphthyl, etc.), which may be fully saturated or contain one or more unsaturation degrees, but no aromatic ring can be present. In one implementation, the cycloalkyl group contains 3-6 carbon atoms, such as C₃₋₆ saturated or partially unsaturated cycloalkyl. Examples of cycloalkyl groups include, but are not limited to, cyclopropyl, cyclobutyl, cyclopentyl, cyclohexyl, cyclopentenyl, cyclohexenyl, and so on. In one embodiment, the saturated or partially unsaturated cycloalkyl group is selected from the group consisting of saturated monocyclic alkyl, saturated bicyclic alkyl, saturated tricyclic alkyl, partially unsaturated monocyclic alkyl, partially unsaturated bicyclic alkyl, and partially unsaturated tricyclic alkyl, C₄₋₇ cycloalkyl represents a cycloalkyl group with 4-7 ring atoms. C₃₋₆ cycloalkyl represents a cycloalkyl group with 3-6 ring atoms.

In this article, the heterocyclyl group referred to, whether as a single group or a part of a complex group, refers to a saturated (i.e., “heterocycloalkyl”) or partially unsaturated monovalent monocyclic or bicyclic group, which has 2, 3, 4, 5, 6, 7, 8, or 9 carbon atoms and one or more (e.g. one, two, three, or four) heteroatoms selected from C(═O), O, S, S(═O), S(═O)₂, and NR′ in the ring, where R′ represents a hydrogen atom or a C₁₋₆alkyl or halo-C₁₋₆alkyl group. The heterocyclyl group can be connected to the rest of the molecule through any one of the carbon atoms or nitrogen atoms (if any). Specifically, a 3-10 membered heterocyclyl group refers to a group with 3-10 (e.g. 3-7, 4-6, or 5-6) carbon atoms and heteroatoms in the ring, such as but not limited to epoxyethane, aziridinyl, azetidine, oxetanyl, tetrahydrofuran, dioxolinyl, pyrrolidinyl, pyrrolidone, imidazolyl, pyrazolyl, pyrrolidinyl, tetrahydropyranyl, piperidinyl, morpholinyl, dithianyl, thiomorpholinyl, piperazinyl, or trithianyl.

In this article, whether as a single group or a part of a complex group, the aryl group refers to an optional group or substituent derived from an aromatic carbon ring. The aryl group can be a monocyclic aryl group (such as a phenyl group) or a polycyclic aryl group. In other words, the aryl group can be a monocyclic aryl group, a fused ring aryl group, two or more monocyclic aryl groups conjugated by a carbon carbon single bond, a monocyclic aryl group and a fused ring aryl group conjugated by a carbon carbon single bond, or two or more fused ring aryl groups conjugated by a carbon single bond, where at least one ring is an aromatic ring. That is, unless otherwise specified, two or more aromatic groups conjugated by carbon carbon single bonds can also be considered as aromatic groups in this application. Wherein fused aromatic groups can include bicyclic fused aromatic groups (such as naphthyl), tricyclic fused aromatic groups (such as phenanthrene, fluorenyl, anthracene), and so on. The aryl group does not contain heteroatoms such as B, N, O, S, P, Se, and Si. Examples of aryl groups may include, but are not limited to, phenyl, naphthyl, fluorenyl, phenylnaphthyl, spirobifluorenyl, anthracene, phenanthrene, biphenyl, terphenyl, triphenylenyl, perylenyl, benzo[9,10]phenanthrenyl, pyrenyl, benzofluoranthenyl, chrysenyl, etc. Aryl groups may represent monocyclic, bicyclic, and tricyclic carbon ring systems containing 6-14 ring atoms, or 6-12 ring atoms, or 6-10 ring atoms, where at least one ring is aromatic and has one or more attachment points connected to the rest of the molecule. Unless otherwise specified, the “C₆₋₁₄ aryl” group represents an aryl group containing 6-14 ring atoms.

In this article, the heteroaryl group, whether as a single group or as a part of a complex group, refers to a monovalent aromatic ring or its derivative containing 1, 2, 3, 4, 5, or 6 heteroatoms in a ring. The heteroatoms can be one or more of B, O, N, P, Si, Se, and S. The heteroaryl group may be a monocyclic or polycyclic heteroaryl group. In other words, the heteroaryl group can be a single aromatic ring system or multiple aromatic ring system conjugated by carbon carbon single bonds, and any aromatic ring system can be an aromatic monocyclic or an aromatic fused ring, with at least one ring being a heteroaromatic ring. For example, heteroaryl groups include, but are not limited to, thienyl, furyl, pyrrolyl, imidazolyl, thiazolyl, oxazolyl, oxadiazole, triazolyl, pyridinyl, bipyridinyl, pyrimidinyl, triazinyl, acridinyl, pyridazinyl, pyrazinyl, quinolinyl, quinazolinyl, quinoxalinyl, phenoxazinyl, phthalocyaninyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothiophenyl, dibenzothiophenyl, thienothienyl, benzofuryl, phenanthrolinyl, isoxazolyl, thiadiazolyl, phenothiazinyl, silicon fluorenyl, dibenzofuryl, as well as N-phenylcarbazolyl, N-pyridylcarbazolyl, N-methylcarbazolyl, etc. Heteroaryl groups may represent monocyclic, bicyclic, and tricyclic ring systems containing 5-14 ring atoms, or 5-12 ring atoms, or 5-10 ring atoms, where at least one ring is aromatic and has one or more attachment points connected to the rest of the molecule. Unless otherwise specified, the 5-14 membered heteroaryl group represents a heteroaryl group containing 5-14 ring atoms.

Cycloalkylene, heterocyclylene, arylene, heteroarylene and alkylene respectively are cycloalkyl, heterocyclyl, aryl, heteroaryl and alkyl groups used as connecting groups in the present invention, they are divalent groups that are connected to the rest of the molecule at two junctions.

The term “halo” refers to fluorine (F), chlorine (Cl), bromine (Br), or iodine (I).

When describing the rest of the molecule, it refers to the rest parts that do not include the substituents of the group itself.

In this article, “multiple” or “more” refers to two or more, such as 2, 3, 4, 5, 6, etc.

The hydrogen atoms in the compound structure of this application include various isotopic atoms of hydrogen element, such as hydrogen (H), deuterium (D), or tritium (T).

Unless otherwise explicitly stated, the description used in the present invention of “each of . . . independently” and “ . . . each independently” and “ . . . independently” can be interchangeable and should be broadly understood. It can refer to specific options expressed by the same symbols in different groups that do not affect each other, or it can refer to specific options expressed by the same symbols in the same group that do not affect each other.

The terms “optional”, “optionally”, or “arbitrary” refer to events or situations that may or may not occur subsequently, and the description includes situations where the event or situation occurs and situations where it does not occur. For example, “optionally substituted by . . . ” refers to whether the substitution may exist or not.

When the terms “independently” and “optionally” are used in combination, for example, “independently and optionally substituted with . . . ” means that specific options are not affected by each other and are substituted . . . or unsubstituted.

The term “unsaturated” refers to a portion containing one or more degree of unsaturation.

In various parts of the present disclosure, linking substituents are described. When the structure clearly requires a linking group, the Markush variables listed for that group should be understood to be the linking group. For example, if the structure requires a linking group, and “alkyl” or “aryl” is listed for the definition of Markush group of this variable, it should be understood that “alkyl” or “aryl” respectively represents the linker: alkylene group or arylene group.

The term “two substituents connected to form a ring” refers to connecting the positions of two substituents through a bond or a long chain of one or more atoms, forming a ring together with the atoms between the positions of the two substituents in the parent molecule.

The term “substituted” refers to one or more hydrogen atoms on a specific group are replaced by specific substituents. The specific substituents are those described in the preceding text or those that appear in various embodiments. Unless otherwise specified, a substituted group may have a substituent selected from a specific group at any substitutable site of the group, which may be the same or different at each position, i.e., the substituents are independent of each other. Skills in the art should understand that the expected combinations of substituents in the present invention are those that are stable or chemically achievable.

The term “room temperature” refers to the temperature of the reaction system being the same as the temperature of the environment, without the need for cooling or heating. In some embodiments, room temperature refers to a temperature of about 10 degrees Celsius to 40 degrees Celsius, and in some embodiments, room temperature refers to a temperature of about 25 degrees Celsius to 35 degrees Celsius.

### DETAILED DESCRIPTION OF THE EMBODIMENTS

To make the objects, technical solutions, and advantages of the disclosure more apparent, the present invention will be described in further detail below with reference to an example. The specific embodiment described hereby is only to explain this disclosure, not intended to limit this disclosure. Additionally, in the following description, depiction on well-known structures and technologies are omitted to avoid unnecessarily confusing the concepts of the present invention. This structure and technology have also been described in many publications.

The reagents used in the present invention can be purchased from the market or prepared by the method described in the present invention.

### Example 1: Preparation of Substrate Phosphine III-1

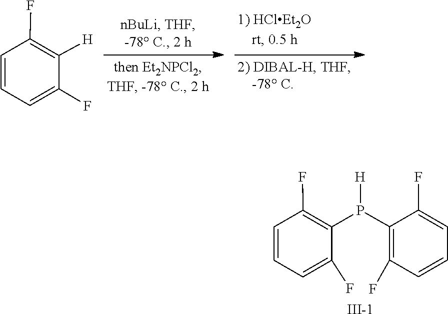

Preparation of III-1: To a 250 mL reaction flask were added 1,3-difluorobenzene (4.78 g, 42 mmol) and 50 mL of tetrahydrofuran solution, then butyl lithium (42 mmol, 2.0 M, 21 mL) was added. After reacting at low temperature for 2 hours, Et₂NPCl (3.48 g, 20 mmol) was added. After 2 hours, the solvent was removed under reduced pressure, and a solution of hydrogen chloride in ether was added. Finally, it was reduced with DIBAL-H. After filtration, the solvent was removed to obtain product III-1, which does not require further purification.

### Example 2: Preparation of Substrate Phosphine III-2

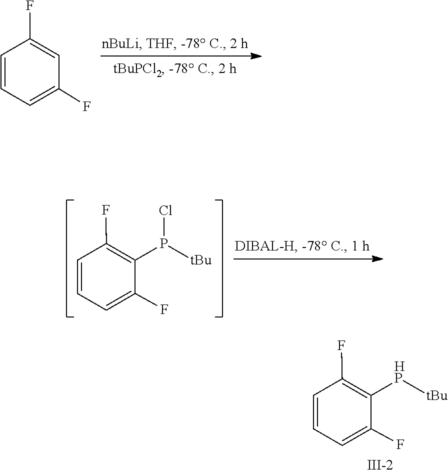

Preparation of III-2: To a 250 mL reaction flask were added 1,3-difluorobenzene (4.78 g, 42 mmol) and 50 mL of tetrahydrofuran solution, then butyl lithium (42 mmol, 2.0 M, 21 mL) was added. After reacting at low temperature for 2 hours, tert-butyldichlorophosphine (3.18 g, 20 mmol) was added. After 2 hours, it was reduced by adding DIBAL-H. After filtration, the solvent was removed to obtain product III-2, which does not require further purification.

### Example 3: Preparation of Compound I-1

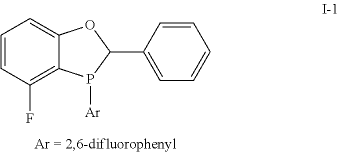

The corresponding substrate phosphine III-1 (0.2 mmol) was dissolved in 2 mL of dry tetrahydrofuran under an argon atmosphere at room temperature. Then, potassium tert butoxide solid (0.3 mmol, 1.5 equiv.) and the corresponding IV-1 benzaldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the target product of formula I-1 was obtained with a yield of 93% and a non enantioselectivity greater than 10:1 as a colorless oily fluid.

### Structural Characterization Data of Product I-1:

¹H NMR (400 MHz, CDCl₃) δ 7.45-7.30 (m, 7H), 6.95-6.83 (m, 3H), 6.69 (td, J=8.0, 3.9 Hz, 1H), 6.48 (s, 1H). ³¹P NMR (162 MHz, CDCl₃) δ −32.30. ¹⁹F NMR (377 MHz, CDCl₃) δ −102.37, −102.61. ¹³C NMR (151 MHz, CDCl₃) δ 166.22, 166.17, 165.65, 165.56, 164.94, 164.89, 164.83, 163.99, 163.90, 163.29, 163.24, 163.18, 140.12, 139.96, 133.74, 133.68, 132.26, 132.22, 132.15, 132.07, 128.67, 127.85, 127.83, 125.15, 125.10, 112.01, 111.98, 111.85, 111.82, 111.78, 111.61, 108.30, 108.15, 107.16, 107.14, 88.17, 88.02, 77.21, 77.00, 76.79. High Resolution Mass Spectro: calculated value: [M+H]⁺: 345.0651; Found value: 345.0649.

### Example 4: Preparation of Compound I-2

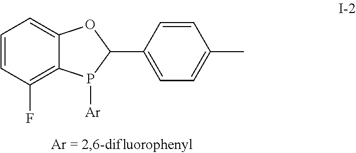

The operation steps are the same as Example 1, except that p-methylbenzaldehyde is used as the substrate to obtain the target product of formula I-2 with a yield of 73%, >20:1 d.r., as a colorless oil.

### Structural Characterization Data of Product I-2:

¹H NMR (600 MHz, CDCl₃) δ 7.37-7.29 (m, 1H), 7.24 (dd, J=8.0, 1.5 Hz, 1H), 7.14 (d, J=8.0 Hz, 1H), 6.90 (d, J=8.2 Hz, 1H), 6.86 (td, J=8.1, 1.5 Hz, 1H), 6.68 (td, J=8.0, 3.8 Hz, 1H), 6.45 (s, 1H), 2.32 (s, 1H). ³¹P NMR (162 MHz, CDCl₃) δ −32.90. ¹⁹F NMR (565 MHz, CDCl₃) δ −102.39-102.65 (m). ¹³C NMR (151 MHz, CDCl₃) δ 166.21, 165.57, 164.91, 164.01, 163.26, 137.68, 137.08, 136.92, 133.66, 133.60, 132.15, 132.08, 132.01, 129.36, 129.35, 125.17, 125.12, 111.99, 111.96, 111.83, 111.80, 108.20, 108.06, 107.16, 107.14, 88.15, 88.01, 77.21, 77.00, 76.79, 21.11. High Resolution Mass Spectro: calculated value: [M+H]⁺: 359.0807; Found value: 359.0803.

### Example 5: Preparation of Compound I-3

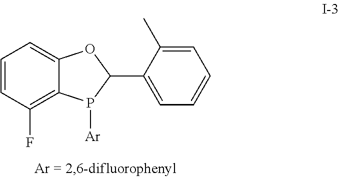

The operation steps are the same as Example 1, except that o-methylbenzaldehyde is used as the substrate to obtain the target product of formula I-3 with a yield of 88%, >20:1 d.r., as a colorless oil.

### Structural Characterization Data of Product I-3:

¹H NMR (600 MHz, CDCl₃) δ 7.41-7.32 (m, 1H), 7.23-7.18 (m, 1H), 7.14 (t, J=7.1 Hz, 1H), 6.95 (d, J=8.2 Hz, 1H), 6.90 (t, J=8.1 Hz, 1H), 6.68 (td, J=8.0, 3.8 Hz, 1H), 6.58 (s, 1H), 2.38 (s, 1H). ³¹P NMR (243 MHz, CDCl₃) δ −37.48. ¹⁹F NMR (565 MHz, CDCl₃) δ −101.47, −103.10. ¹³C NMR (151 MHz, CDCl₃) δ 166.91, 166.86, 165.54, 165.48, 165.42, 165.26, 165.17, 163.89, 163.83, 163.77, 163.61, 163.52, 137.93, 137.79, 134.11, 134.08, 133.69, 133.63, 132.86, 132.79, 132.71, 130.80, 130.79, 127.72, 127.70, 126.24, 126.22, 124.23, 124.19, 112.05, 112.02, 111.89, 111.86, 108.16, 108.02, 106.96, 106.94, 85.61, 85.59, 85.57, 85.46, 85.44, 77.21, 77.00, 76.79, 19.88, 19.84. High Resolution Mass Spectro: calculated value: [M+H]⁺: 359.0807; Found value: 359.0805.

### Example 6: Preparation of Compound I-4

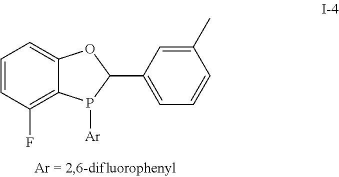

The operation steps are the same as Example 1, except that m-methylbenzaldehyde is used as the substrate to obtain the target product of formula I-4 with a yield of 69%, >20:1 d.r., as a colorless oil.

### Structural Characterization Data of Product I-4:

¹H NMR (600 MHz, CDCl₃) δ 7.40-7.29 (m, 2H), 7.23 (t, J=7.6 Hz, 1H), 7.18-7.13 (m, 2H), 7.09 (d, J=7.5 Hz, 1H), 6.92 (d, J=8.2 Hz, 1H), 6.88 (td, J=8.0, 1.7 Hz, 2H), 6.69 (td, J=8.0, 3.9 Hz, 1H), 6.45 (s, 1H), 2.34 (s, 3H). ³¹P NMR (243 MHz, CDCl₃) δ −32.52 (m). ¹⁹F NMR (565 MHz, CDCl₃) δ −102.48 (m), −102.55 (m). ¹³C NMR (151 MHz, CDCl₃) δ 166.30, 166.25, 165.65, 165.56, 165.02, 164.96, 164.91, 163.99, 163.90, 163.37, 163.31, 163.26, 140.11, 139.96, 138.49, 133.73, 133.66, 132.25, 132.18, 132.10, 128.72, 128.70, 128.63, 125.83, 125.78, 122.28, 122.23, 112.03, 112.00, 111.88, 111.85, 108.26, 108.11, 107.19, 107.16, 88.26, 88.23, 88.21, 88.11, 88.09, 88.07, 77.25, 77.04, 76.83, 21.53. High Resolution Mass Spectro: calculated value: [M+H]⁺: 359.0807; Found value: 359.0805.

### Example 7: Preparation of Compound I-5

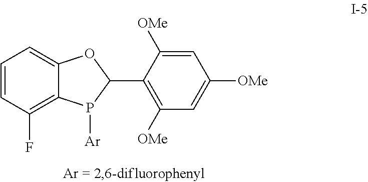

The operation steps are the same as Example 1, except that 2,4,6-trimethoxybenzaldehyde is used as the substrate to obtain the target product of formula I-5 with a yield of 46%, >20:1 d.r., as a white solid.

### Structural Characterization Data of Product I-5:

¹H NMR (600 MHz, CDCl₃) δ 7.35-7.27 (m, 1H), 7.21 (dd, J=14.5, 8.1 Hz, 1H), 6.88-6.80 (m, 3H), 6.65 (d, J=8.1 Hz, 1H), 6.57 (td, J=8.0, 3.9 Hz, 1H), 6.11 (s, 2H), 3.80 (s, 3H), 3.66 (s, 6H). ³¹P NMR (243 MHz, CDCl₃) δ −32.48-47.61 (m). ¹⁹F NMR (565 MHz, CDCl₃) δ −101.89 (m), −105.10-−107.50 (m). ¹³C NMR (151 MHz, CDCl₃) δ 166.94, 165.73, 164.07, 161.87, 159.47, 132.15, 132.09, 111.80, 111.65, 111.62, 109.41, 109.27, 106.49, 106.35, 106.00, 105.99, 90.91, 79.15, 79.02, 77.21, 77.00, 76.79, 55.70, 55.39. High Resolution Mass Spectro: calculated value: [M+H]⁺: 435.0968; Found value: 435.0966.

### Example 8: Preparation of Compound I-6

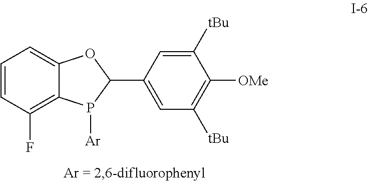

The operation steps are the same as Example 1, except that 3,5-di-tert-butyl-4-methoxybenzaldehyde is used as the substrate to obtain the target product of formula I-6 with a yield of 78%, >20:1 d.r., as a white solid.

### Structural Characterization Data of Product I-6:

¹H NMR (400 MHz, CDCl₃) δ 7.33 (qd, J=8.2, 6.9 Hz, 1H), 7.22 (d, J=1.7 Hz, 1H), 6.94-6.82 (m, 1H), 6.68 (td, J=8.0, 3.9 Hz, 1H), 6.42 (s, 1H), 3.67 (s, 1H), 1.38 (s, 9H). ³¹P NMR (162 MHz, CDCl₃) δ −33.97 (m). ¹⁹F NMR (377 MHz, CDCl₃) δ −102.57 (m). ¹³C NMR (151 MHz, CDCl₃) δ 166.23, 165.55, 164.92, 163.99, 163.27, 159.18, 159.15, 143.90, 133.59, 133.54, 133.40, 132.05, 131.98, 131.91, 123.62, 123.56, 111.98, 111.95, 111.83, 111.80, 108.13, 107.99, 107.06, 107.04, 88.57, 88.43, 77.21, 77.00, 76.79, 64.22, 35.80, 31.93. High Resolution Mass Spectro: calculated value: [M+H]⁺: 487.2008; Found value: 487.2010.

### Example 9: Preparation of Compound I-7

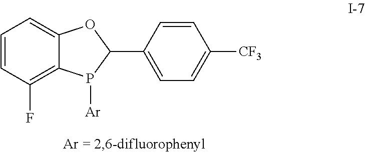

The operation steps are the same as Example 1, except that 4-trifluoromethylbenzaldehyde is used as the substrate to obtain the target product of formula I-7 with a yield of 33%, >5:1 d.r., as a colorless oil.

### Structural Characterization Data of Product I-7:

¹H NMR (600 MHz, CDCl₃) δ 7.60 (d, J=8.2 Hz, 2H), 7.48 (d, J=7.8 Hz, 2H), 7.37 (dq, J=23.3, 7.4, 6.7 Hz, 2H), 6.95 (d, J=8.2 Hz, 1H), 6.89 (t, J=7.5 Hz, 2H), 6.72 (td, J=8.0, 3.9 Hz, 1H), 6.51 (s, 1H). ³¹P NMR (243 MHz, CDCl₃) δ −30.37 (m). ¹⁹F NMR (565 MHz, CDCl₃) δ −62.60, −101.71-−102.20 (m), −102.37-−102.95 (m). ¹³C NMR (151 MHz, CDCl₃) δ 165.95, 164.86, 163.20, 144.29, 134.07, 134.01, 132.52, 132.44, 132.37, 125.70, 125.50, 125.45, 112.13, 112.11, 111.98, 111.95, 108.72, 108.58, 107.26, 87.58, 87.42, 77.24, 77.02, 76.81, 14.21. High Resolution Mass Spectro: calculated value: [M+H]⁺: 413.0524; Found value: 413.0522.

### Example 10: Preparation of Compound I-8

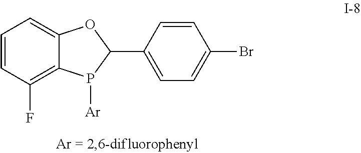

The operation steps are the same as Example 1, except that 4-bromobenzaldehyde is used as the substrate to obtain the target product of formula I-8 with a yield of 79%, >10:1 d.r., as a colorless oil.

### Structural Characterization Data of Product I-8:

¹H NMR (600 MHz, CDCl₃) δ 7.46 (d, J=8.3 Hz, 1H), 7.41-7.29 (m, 1H), 7.24 (d, J=7.6 Hz, 1H), 6.91 (d, J=8.2 Hz, 1H), 6.88 (t, J=8.1 Hz, 1H), 6.70 (td, J=8.0, 3.9 Hz, 1H), 6.42 (s, 1H). ³¹P NMR (162 MHz, CDCl₃) δ −31.54. ¹⁹F NMR (565 MHz, CDCl₃) δ −102.12, −102.6. ¹³C NMR (151 MHz, CDCl₃) δ 165.89, 165.55, 164.84, 163.98, 163.19, 139.20, 139.05, 133.91, 133.85, 132.37, 132.29, 132.23, 131.77, 126.92, 126.86, 121.72, 121.70, 112.06, 112.03, 111.91, 111.88, 108.53, 108.39, 107.21, 107.19, 87.59, 87.44, 77.21, 77.00, 76.79. High Resolution Mass Spectro: calculated value: [M+H]⁺: 422.9756; Found value: 422.9753.

### Example 11: Preparation of Compound I-9

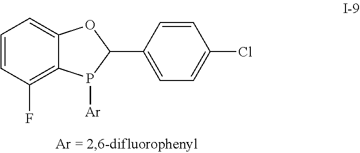

The operation steps are the same as Example 1, except that 4-chlorobenzaldehyde is used as the substrate to obtain the target product of formula I-9 with a yield of 83%, >20:1 d.r., as a colorless oil.

### Structural Characterization Data of Product I-9:

¹H NMR (600 MHz, CDCl₃) δ 7.40-7.28 (m, 1H), 6.92 (d, J=8.1 Hz, 1H), 6.88 (td, J=8.2, 1.6 Hz, 1H), 6.71 (td, J=8.0, 3.9 Hz, 1H), 6.45 (s, 1H). ³¹P NMR (243 MHz, CDCl₃) δ −26.68-−34.98 (m). ¹⁹F NMR (565 MHz, CDCl₃) δ −101.96-−102.78 (m). ¹³C NMR (151 MHz, CDCl₃) δ 165.96, 165.91, 165.64, 165.55, 164.89, 164.84, 163.98, 163.89, 163.25, 163.19, 163.13, 138.66, 138.50, 133.90, 133.84, 133.63, 133.60, 132.36, 132.29, 132.21, 128.84, 128.83, 126.60, 126.54, 112.06, 112.03, 111.90, 111.87, 108.52, 108.37, 107.21, 107.19, 87.56, 87.41, 77.21, 77.00, 76.79. High Resolution Mass Spectro: calculated value: [M+H]⁺: 379.0261; Found value: 379.0259.

### Example 12 Preparation of Compounds I-10 to I-22

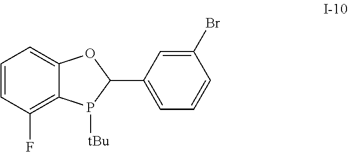 [SMILES: CC(C)(C)P1c2c(F)cccc2OC1c1cccc(Br)c1]

The corresponding substrate phosphine III-2 (0.2 mmol) was dissolved in 2 mL of dry tetrahydrofuran under an argon atmosphere at room temperature. Then, potassium tert butoxide solid (0.3 mmol, 1.5 equiv.) and the corresponding 3-bromobenzaldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-50:1) as the eluent, and the target product of formula I-10 was obtained with a yield of 84% and a non enantioselectivity greater than 20:1 as a colorless oily fluid.

### Structural Characterization Data of Product I-10:

¹H NMR (600 MHz, CDCl₃) δ 7.38 (s, 1H), 7.37-7.28 (m, 2H), 7.20-7.12 (m, 2H), 6.89 (d, J=8.1 Hz, 1H), 6.70 (td, J=8.0, 3.2 Hz, 1H), 5.95 (s, 1H), 1.11 (d, J=12.7 Hz, 9H). ³¹P NMR (162 MHz, CDCl₃) δ 13.53. ¹⁹F NMR (376 MHz, CDCl₃) δ −102.78. ¹³C NMR (101 MHz, CDCl₃) δ 165.47 (d, J=8.6 Hz), 164.44 (dd, J=247.8, 12.4 Hz), 143.56 (d, J=17.2 Hz), 133.02 (d, J=9.2 Hz), 130.41 (d, J=2.4 Hz), 130.11 (d, J=1.3 Hz), 127.88 (d, J=7.1 Hz), 123.53 (d, J=7.6 Hz), 122.67 (d, J=1.5 Hz), 108.92 (dd, J=27.6, 20.6 Hz), 108.29 (dd, J=22.8, 1.8 Hz), 107.03 (d, J=3.4 Hz), 84.08 (d, J=28.5 Hz), 32.10 (d, J=20.5 Hz), 26.79 (d, J=14.6 Hz, 3C). High Resolution Mass Spectro: calculated value: [M+H]⁺: 367.0257; Found value: 367.0261.

### Preparation of Product I-11:

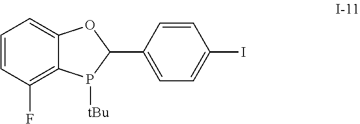 [SMILES: CC(C)(C)P1c2c(F)cccc2OC1c1ccc(I)cc1]

The operation steps are the same as Example 2, except that 4-iodobenzaldehyde is used as the substrate to obtain the target product of formula I-11 with a yield of 79%, >20:1 d.r., as a colorless oil.

¹H NMR (400 MHz, CDCl₃) δ 7.62 (d, J=8.4 Hz, 2H), 7.31 (td, J=8.1, 6.1 Hz, 1H), 7.03-6.97 (m, 2H), 6.87 (d, J=8.1 Hz, 1H), 6.69 (td, J=8.0, 3.2 Hz, 1H), 5.93 (s, 1H), 1.10 (d, J=12.8 Hz, 9H). ³¹P NMR (162 MHz, CDCl₃) δ 13.10. ¹⁹F NMR (376 MHz, CDCl₃) δ −102.76. ¹³C NMR (101 MHz, CDCl₃) δ 165.48 (d, J=8.7 Hz), 164.46 (dd, J=247.7, 12.2 Hz), 140.94 (d, J=17.1 Hz), 137.57 (d, J=1.4 Hz, 2C), 132.97 (d, J=9.2 Hz), 126.84 (d, J=7.3 Hz, 2C), 108.96 (dd, J=27.5, 20.6 Hz), 108.23 (dd, J=22.7, 1.8 Hz), 106.96 (d, J=3.4 Hz), 92.60 (d, J=3.5 Hz), 84.32 (d, J=28.1 Hz), 32.07 (d, J=20.8 Hz), 26.79 (d, J=14.5 Hz, 3C). High Resolution Mass Spectro: calculated value: [M+H]⁺: 415.0118; Found value: 415.0122.

### Preparation of Product I-12:

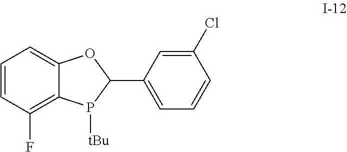 [SMILES: CC(C)(C)P1c2c(F)cccc2OC1c1cccc(Cl)c1]

The operation steps are the same as Example 2, except that 3-chlorobenzaldehyde is used as the substrate to obtain the target product of formula I-12 with a yield of 50%, >20:1 d.r., as a colorless oil.

### Structural Characterization Data of Product I-12:

¹H NMR (400 MHz, CDCl₃) δ 7.34 (td, J=8.3, 6.3 Hz, 1H), 7.23-7.10 (m, 1H), 7.07 (dt, J=7.5, 2.1 Hz, OH), 6.92 (dd, J=8.1, 0.8 Hz, OH), 6.75-6.65 (m, OH), 6.29 (s, OH), 1.14 (d, J=12.4 Hz, 5H). ³¹P NMR (162 MHz, CDCl₃) δ 10.73. ¹⁹F NMR (376 MHz, CDCl₃) δ −102.65. ¹³C NMR (101 MHz, CDCl₃) δ 166.19, 166.10, 166.03, 165.91, 163.57, 163.44, 138.06, 137.92, 133.00, 132.91, 130.27, 130.21, 129.75, 129.73, 128.45, 128.42, 127.00, 126.98, 125.92, 125.88, 109.68, 109.47, 109.40, 109.19, 108.47, 108.45, 108.24, 108.22, 106.81, 106.77, 82.71, 82.42, 33.31, 33.09, 27.19, 27.05. High Resolution Mass Spectro: calculated value: [M+H]⁺: 323.0762; Found value: 323.0761.

### Preparation of Product I-13:

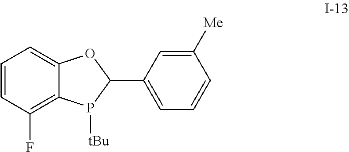 [SMILES: Cc1cccc(C2Oc3cccc(F)c3P2C(C)(C)C)c1]

The operation steps are the same as Example 2, except that 3-methylbenzaldehyde is used as the substrate to obtain the target product of formula I-13 with a yield of 86%, >20:1 d.r., as a colorless oil.

### Structural Characterization Data of Product I-13:

¹H NMR (400 MHz, CDCl₃) δ 7.31 (td, J=8.2, 6.1 Hz, 1H), 7.23-7.12 (m, 1H), 7.08-7.01 (m, 3H), 6.88 (dd, J=8.1, 0.8 Hz, 1H), 6.69 (tdd, J=8.0, 3.2, 0.8 Hz, 1H), 5.97 (s, 1H), 2.32 (s, 3H), 1.11 (d, J=12.6 Hz, 9H). ³¹P NMR (162 MHz, CDCl₃) δ 12.04. ¹⁹F NMR (376 MHz, CDCl₃) δ −102.97. ¹³C NMR (101 MHz, CDCl₃) δ 165.82 (d, J=8.4 Hz), 165.75-163.11 (m), 141.04 (d, J=16.7 Hz), 138.30 (d, J=1.4 Hz), 132.76 (d, J=9.1 Hz), 128.50 (d, J=1.4 Hz), 128.19 (d, J=2.5 Hz), 125.50 (d, J=7.4 Hz), 121.97 (d, J=7.0 Hz), 109.32 (dd, J=27.6, 20.7 Hz), 107.92 (dd, J=22.8, 1.8 Hz), 106.92 (d, J=3.3 Hz), 84.92 (d, J=27.6 Hz), 31.98 (d, J=20.9 Hz), 26.82 (d, J=14.5 Hz, 3C), 21.48. High Resolution Mass Spectro: calculated value: [M+H]⁺: 303.1309; Found value: 303.1312.

### Preparation of Product I-14:

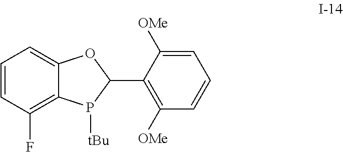 [SMILES: COc1cccc(OC)c1C1Oc2cccc(F)c2P1C(C)(C)C]

The operation steps are the same as Example 2, except that 2,6-dimethoxybenzaldehyde is used as the substrate to obtain the target product of formula I-14 with a yield of 91%, >20:1 d.r., as a white solid.

### Structural Characterization Data of Product I-14:

¹H NMR (400 MHz, CDCl₃) δ 7.30-7.11 (m, 2H), 6.67-6.56 (m, 3H), 6.53 (d, J=8.3 Hz, 2H), 3.65 (s, 5H), 1.08 (d, J=12.3 Hz, 9H). ³¹P NMR (162 MHz, CDCl₃) δ 11.51. ¹⁹F NMR (376 MHz, CDCl₃) δ −104.35. ¹³C NMR (101 MHz, CDCl₃) δ 166.72 (d, J=9.5 Hz), 163.29 (dd, J=244.5, 12.1 Hz), 158.45, 131.42 (d, J=9.3 Hz), 129.61 (d, J=1.8 Hz, 2C), 117.04 (d, J=16.0 Hz), 111.89 (dd, J=27.8, 18.1 Hz), 106.37 (dd, J=23.2, 1.9 Hz), 106.01 (d, J=3.0 Hz), 104.18, 75.99 (d, J=24.7 Hz, 2C), 55.66 (s, 2C), 31.49 (d, J=18.5 Hz), 26.77 (dd, J=14.7, 1.5 Hz, 3C). High Resolution Mass Spectro: calculated value: [M+H]⁺: 349.1363; Found value: 349.1366.

### Preparation of Product I-15:

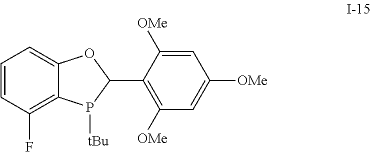 [SMILES: COc1cc(OC)c(C2Oc3cccc(F)c3P2C(C)(C)C)c(OC)c1]

The operation steps are the same as Example 2, that 2,4,6-trimethoxybenzaldehyde is used as the substrate to obtain the target product of formula I-15 with a yield of 93%, >20:1 d.r., as a white solid.

### Structural Characterization Data of Product I-15:

¹H NMR (400 MHz, CDCl₃) δ 7.18 (tdd, J=7.9, 6.2, 1.3 Hz, 1H), 6.61 (m, 2H), 6.49 (d, J=3.8 Hz, 1H), 6.10 (s, 2H), 3.79 (s, 3H), 3.64 (s, 6H), 1.06 (d, J=12.2 Hz, 9H). ³¹P NMR (162 MHz, CDCl₃) δ 10.67. ¹⁹F NMR (376 MHz, CDCl₃) δ −104.43. ¹³C NMR (101 MHz, CDCl₃) δ 166.58 (d, J=9.5 Hz), 163.29 (dd, J=244.3, 12.0 Hz), 161.41 (d, J=1.7 Hz), 159.23, 131.33 (d, J=9.3 Hz, 2C), 111.92 (dd, J=27.9, 18.2 Hz), 109.92 (d, J=16.1 Hz), 106.24 (dd, J=23.3, 1.9 Hz), 105.98 (d, J=3.0 Hz), 90.82 (s, 2C), 75.89 (d, J=24.0 Hz), 55.58 (s, 2C), 55.29, 31.37 (d, J=18.5 Hz), 26.73 (dd, J=14.8, 1.4 Hz, 3C). High Resolution Mass Spectro: calculated value: [M+H]⁺: 379.1469; Found value: 379.1471.

### Preparation of Compound I-16:

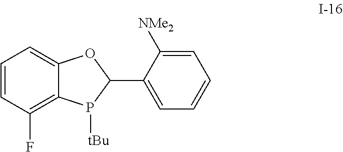 [SMILES: CN(C)c1ccccc1C1Oc2cccc(F)c2P1C(C)(C)C]

The corresponding substrate phosphine III-2 (0.2 mmol) was dissolved in 2 mL of dry tetrahydrofuran under an argon atmosphere at room temperature. Then, potassium tert butoxide solid (0.3 mmol, 1.5 equiv.) and the corresponding 2-dimethylaminobenzaldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-50:1) as the eluent, and the target product of formula I-16 was obtained with a yield of 79% and a non enantioselectivity greater than 5:1 as a colorless oil.

### Structural Characterization Data of Product I-16:

¹H NMR (400 MHz, CDCl₃) δ7.31-7.18 (m, 3H), 7.10-7.05 (m, 1H), 7.00 (td, J=7.9, 7.0, 2.2 Hz, 1H), 6.87 (dd, J=8.1, 0.8 Hz, 1H), 6.68-6.61 (m, 1H), 6.31 (d, J=1.8 Hz, 1H), 2.66 (s, 6H), 1.11 (d, J=12.0 Hz, 9H). ³¹P NMR (162 MHz, CDCl₃) δ 13.78. ¹⁹F NMR (377 MHz, CDCl₃) δ −103.23. ¹³C NMR (100 MHz, CDCl₃) δ 166.48, 166.39, 165.90, 165.78, 163.45, 163.33, 150.95, 150.91, 137.29, 137.15, 132.41, 132.32, 128.09, 128.06, 125.11, 125.06, 124.95, 124.94, 121.75, 121.73, 110.95, 110.73, 110.67, 110.45, 107.82, 107.80, 107.59, 107.57, 106.82, 106.78, 82.27, 82.00, 77.32, 77.00, 76.68, 45.31, 45.29, 32.60, 32.37. High Resolution Mass Spectro: calculated value: [M+H]⁺: 332.1574; Found value: 332.1576.

### Preparation of Compound I-17:

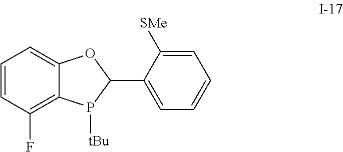 [SMILES: CSc1ccccc1C1Oc2cccc(F)c2P1C(C)(C)C]

The corresponding substrate phosphine III-2 (0.2 mmol) was dissolved in 2 mL of dry tetrahydrofuran under an argon atmosphere at room temperature. Then, potassium tert butoxide solid (0.3 mmol, 1.5 equiv.) and the corresponding 2-mercaptobenzaldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-50:1) as the eluent, and the target product of formula I-17 was obtained with a yield of 93% and a non enantioselectivity greater than 20:1 as a white solid.

### Structural Characterization Data of Product I-17:

¹H NMR (400 MHz, CDCl₃) δ 7.36-7.27 (m, 2H), 7.20 (tt, J=8.0, 1.8 Hz, 1H), 7.07-6.99 (m, 2H), 6.91 (d, J=8.6 Hz, 1H), 6.68 (td, J=8.0, 3.1 Hz, 1H), 6.36 (s, 1H), 2.54 (s, 3H), 1.14 (d, J=12.3 Hz, 9H). ³¹P NMR (162 MHz, CDCl₃) δ 9.67. ¹⁹F NMR (377 MHz, CDCl₃) δ −102.76. ¹³C NMR (100 MHz, CDCl₃) δ166.28, 166.19, 166.01, 165.88, 163.55, 163.42, 139.07, 138.92, 133.89, 133.84, 132.82, 132.73, 127.84, 127.82, 127.61, 127.59, 125.67, 125.65, 124.78, 124.73, 110.07, 109.86, 109.79, 109.58, 108.23, 108.22, 108.01, 107.99, 106.72, 106.69, 82.88, 82.60, 77.32, 77.00, 76.68, 33.47, 33.45, 33.24, 33.23, 27.38, 27.24, 17.07, 17.04. High Resolution Mass Spectro: calculated value: [M+H]⁺: 335.1029; Found value: 335.1028.

### Preparation of Compound I-18:

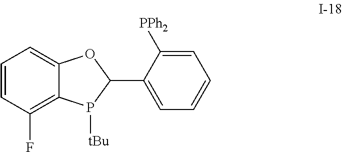

The operation steps are the same as Example 2, except that 2-diphenylphosphono benzaldehyde is used as the substrate to obtain the target product of formula I-18 with a yield of 76%, >20:1 d.r., as a white solid.

### Structural Characterization Data of Product I-18:

¹H NMR (400 MHz, CDCl₃) δ 7.38-7.22 (m, 11H), 7.20 (dd, J=7.2, 1.4 Hz, 1H), 7.17-7.09 (m, 2H), 6.94 (dt, J=8.9, 3.0 Hz, 1H), 6.83-6.75 (m, 2H), 6.68 (td, J=7.9, 3.1 Hz, 1H), 0.98 (d, J=12.4 Hz, 9H). ³¹P NMR (162 MHz, CDCl₃) δ 10.45 (d, J=20.8 Hz), −19.65 (d, J=20.6 Hz). ¹⁹F NMR (376 MHz, CDCl₃) δ −102.69. ¹³C NMR (101 MHz, CDCl₃) δ 166.46, 166.37, 165.91, 165.79, 163.45, 163.33, 145.84, 145.69, 145.61, 145.46, 136.56, 136.45, 135.89, 135.80, 134.70, 134.00, 133.84, 133.82, 133.64, 132.73, 132.64, 132.60, 132.56, 132.46, 132.42, 129.59, 129.57, 128.80, 128.64, 128.58, 128.53, 128.46, 127.89, 127.86, 125.48, 125.42, 125.37, 110.41, 110.17, 109.90, 108.05, 108.03, 107.82, 107.80, 106.73, 106.69, 83.60, 83.32, 83.03, 77.32, 77.00, 76.68, 33.36, 33.13, 27.26, 27.23, 27.12, 27.09. High Resolution Mass Spectro: calculated value: [M+H]⁺: 473.1594; Found value: 473.1595.

### Preparation of Product I-19:

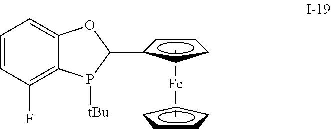

The operation steps are the same as Example 2, except that ferrocenecarboxaldehyde is used as the substrate to obtain the target product of formula I-19 with a yield of 66%, >10:1 d.r., as a yellow oil.

### Structural Characterization Data of Product I-19:

¹H NMR (400 MHz, CDCl₃) δ 7.26 (td, J=8.1, 6.2 Hz, 1H), 6.75 (d, J=7.3 Hz, 1H), 6.66 (td, J=8.8, 3.3 Hz, 1H), 5.73 (s, 1H), 4.17 (m, 4H), 4.09 (s, 5H), 1.09 (d, J=12.5 Hz, 9H). ³¹P NMR (162 MHz, CDCl₃) δ 0.58. ¹⁹F NMR (376 MHz, CDCl₃) δ −103.56 (d, J=1.9 Hz). ¹³C NMR (101 MHz, CDCl₃) δ 165.28 (d, J=8.7 Hz), 164.28 (dd, J=246.8, 12.5 Hz), 132.59 (d, J=9.2 Hz), 110.21 (dd, J=27.4, 18.8 Hz), 107.44 (dd, J=22.9, 1.9 Hz), 107.11 (d, J=3.3 Hz), 88.25 (d, J=21.4 Hz), 82.21 (d, J=26.6 Hz), 68.77 (d, J=1.1 Hz, 5C), 68.37 (dd, J=14.2, 1.3 Hz, 2C), 67.09 (dd, J=111.5, 8.6 Hz, 2C), 31.75 (d, J=18.8 Hz), 26.93 (d, J=14.5 Hz, 3C). High Resolution Mass Spectro: calculated value: [M+H]⁺: 397.0814; Found value: 397.0811.

### Preparation of Product I-20:

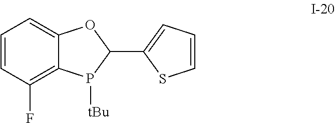 [SMILES: CC(C)(C)P1c2c(F)cccc2OC1c1cccs1]

The operation steps are the same as Example 2, except that dithiophene formaldehyde is used as the substrate to obtain the target product of formula I-20 with a yield of 90%, >20:1 d.r., as a colorless oil.

### Structural Characterization Data of Product I-20:

¹H NMR (400 MHz, CDCl₃) δ 7.27 (m, 1H), 7.21 (dd, J=5.1, 1.2 Hz, 1H), 7.13-7.05 (m, 1H), 6.93 (ddd, J=4.9, 3.5, 1.1 Hz, 1H), 6.79 (dd, J=8.2, 0.8 Hz, 1H), 6.68 (tdd, J=8.0, 3.3, 0.8 Hz, 1H), 6.15 (s, 1H), 1.08 (d, J=12.7 Hz, 9H). ³¹P NMR (162 MHz, CDCl₃) δ 9.51. ¹⁹F NMR (376 MHz, CDCl₃) δ −103.02. ¹³C NMR (101 MHz, CDCl₃) δ 164.63 (d, J=8.6 Hz), 164.33 (dd, J=247.5, 12.5 Hz), 144.26 (d, J=21.8 Hz), 132.85 (d, J=9.1 Hz), 126.82, 125.42 (d, J=3.2 Hz), 124.80 (d, J=10.7 Hz), 109.56 (dd, J=27.5, 19.9 Hz), 108.05 (dd, J=22.7, 1.9 Hz), 107.46 (d, J=3.3 Hz), 80.97 (d, J=26.9 Hz), 32.02 (d, J=20.1 Hz), 26.81 (d, J=14.3 Hz, 3C). High Resolution Mass Spectro: calculated value: [M+H]⁺: 295.0716; Found value: 295.0719.

### Preparation of Product I-21:

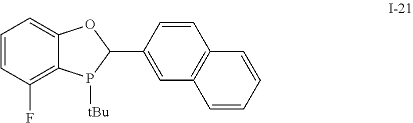 [SMILES: CC(C)(C)P1c2c(F)cccc2OC1c1ccc2ccccc2c1]

The operation steps are the same as Example 2, except that 2-naphthaldehyde is used as the substrate to obtain the target product of formula I-21 with a yield of 87%, >20:1 d.r., as a white solid.

### Structural Characterization Data of Product I-21:

¹H NMR (400 MHz, CDCl₃) § 7.84-7.74 (m, 3H), 7.70 (s, 1H), 7.50-7.40 (m, 2H), 7.40-7.27 (m, 2H), 6.96 (dd, J=8.1, 0.7 Hz, 1H), 6.71 (td, J=7.7, 2.5 Hz, 1H), 6.18 (s, 1H), 1.16 (d, J=12.6 Hz, 9H). ³¹P NMR (162 MHz, CDCl₃) δ 11.43. ¹⁹F NMR (376 MHz, CDCl₃) δ −102.87. ¹³C NMR (101 MHz, CDCl₃) δ 165.83 (d, J=8.7 Hz), 164.53 (dd, J=247.5, 12.2 Hz), 138.31 (d, J=16.7 Hz), 133.17, 132.87 (d, J=9.2 Hz), 132.71 (d, J=1.8 Hz), 128.56, 127.92, 127.66, 126.26, 125.89, 123.31 (d, J=3.1 Hz), 123.24 (d, J=3.2 Hz), 109.22 (dd, J=27.5, 20.6 Hz), 108.08 (dd, J=22.9, 1.8 Hz), 107.00 (d, J=3.3 Hz), 85.12 (d, J=27.7 Hz), 32.11 (d, J=21.4 Hz), 26.86 (d, J=14.6 Hz, 3C). High Resolution Mass Spectro: calculated value: [M+H]⁺: 339.1309; Found value: 339.1308.

### Preparation of Product I-22:

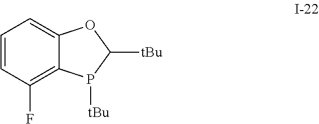 [SMILES: CC(C)(C)C1Oc2cccc(F)c2P1C(C)(C)C]

The operation steps are the same as Example 2, except that tevaleraldehyde is used as the substrate to obtain the target product of formula I-22 with a yield of 92%, >20:1 d.r., as a colorless oil.

### Structural Characterization Data of Product I-22:

¹H NMR (600 MHz, CDCl₃) δ 7.20 (td, J=8.1, 6.2 Hz, 1H), 6.69 (dd, J=8.1, 0.8 Hz, 1H), 6.58 (tdd, J=8.0, 3.3, 0.8 Hz, 1H), 4.57 (d, J=1.6 Hz, 1H), 1.00 (d, J=12.6 Hz, 9H), 0.98 (s, 9H). ³¹P NMR (243 MHz, CDCl₃) δ −9.70. ¹⁹F NMR (377 MHz, CDCl₃) δ −103.33. ¹³C NMR (151 MHz, CDCl₃) δ 166.76, 166.70, 164.83, 164.74, 163.19, 163.11, 132.28, 132.21, 110.14, 110.03, 109.96, 109.85, 107.05, 107.04, 106.90, 106.89, 106.32, 106.30, 93.93, 93.73, 77.23, 77.02, 76.81, 36.19, 36.08, 30.97, 30.85, 26.87, 26.77, 25.95, 25.89. High Resolution Mass Spectro: calculated value: [M+H]⁺: 269.1465; Found value: 269.1464.

### Example 13: Preparation of Compound II-1

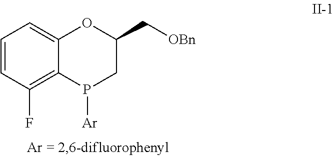

The corresponding substrate phosphine III-1 (0.2 mmol) was dissolved in 2 mL of dry tetrahydrofuran under an argon atmosphere at room temperature. Then, potassium tert butoxide solid (0.3 mmol, 1.5 equiv.) and the corresponding (R)-benzyloxymethyl epoxyethane (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-10:1) as the eluent, and the target product of formula II-1 was obtained with a yield of 86% and a non enantioselectivity greater than 20:1 as a colorless oily fluid.

### Structural Characterization Data of Product II-1:

¹H NMR (400 MHz, CDCl₃) δ 7.39-7.28 (m, 7H), 7.14 (tdd, J=8.3, 6.9, 1.5 Hz, 1H), 6.84 (td, J=8.2, 1.7 Hz, 2H), 6.72 (d, J=8.2 Hz, 1H), 6.57-6.50 (m, 1H), 4.63 (s, 2H), 4.60 (d, J=2.9 Hz, 1H), 4.49-4.34 (m, 1H), 3.80 (ddd, J=10.3, 5.6, 0.8 Hz, 1H), 3.71-3.62 (m, 1H), 2.62 (ddd, J=13.3, 11.7, 6.6 Hz, 1H), 2.30 (ddd, J=20.0, 13.3, 1.1 Hz, 1H). ³¹P NMR (162 MHz, CDCl₃) δ −74.51 (q, J=42.1 Hz). ¹⁹F NMR (377 MHz, CDCl₃) δ −101.14 (d, J=43.1 Hz), −104.99 (d, J=41.5 Hz). ¹³C NMR (151 MHz, CDCl₃) δ 171.15, 166.22, 164.51, 162.87, 159.99, 137.86, 132.40, 130.03, 129.96, 128.43, 127.77, 127.71, 113.60, 111.79, 111.61, 107.53, 107.38, 77.21, 77.00, 76.79, 74.70, 74.67, 73.50, 73.18, 73.12, 60.38, 21.02, 20.56, 14.17. High Resolution Mass Spectro: calculated value: [M+H]⁺: 403.1069; Found value: 403.1066.

### Example 14: Preparation of Compound II-2

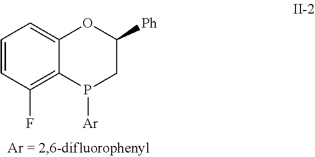

The operation steps are the same as Example 3, except that (R)-phenyl oxirane is used as the substrate to obtain the target product of formula II-2 with a yield of 46%, 2:1 d.r., as a colorless oil.

### Structural Characterization Data of Product II-2:

¹H NMR (600 MHz, CDCl₃) δ 7.48 (d, J=7.7 Hz, 2H), 7.43-7.37 (m, 2H), 7.37-7.29 (m, 2H), 7.17 (td, J=8.3, 6.7 Hz, 1H), 6.89-6.82 (m, 2H), 6.77 (d, J=8.2 Hz, 1H), 6.59 (td, J=8.5, 3.1 Hz, 1H), 5.25 (t, J=11.7 Hz, 1H), 3.37-2.85 (m, 1H), 2.43 (dd, J=20.7, 13.4 Hz, 1H). ³¹P NMR (243 MHz, CDCl₃) δ −72.18 (m). ¹⁹F NMR (565 MHz, CDCl₃) δ −101.06 (m), −104.80 (m). ¹³C NMR (151 MHz, CDCl₃) δ 166.22, 166.16, 166.10, 164.64, 164.56, 164.51, 164.45, 163.03, 162.95, 160.74, 160.69, 141.42, 141.37, 132.53, 132.46, 132.39, 130.16, 130.09, 128.89, 128.82, 128.67, 128.27, 128.18, 128.11, 125.85, 113.80, 113.78, 111.81, 111.78, 111.65, 111.63, 107.64, 107.49, 77.55, 77.51, 26.00, 25.97. High Resolution Mass Spectro: calculated value: [M+H]⁺: 359.0807; Found value: 359.0809.

### Example 15: Preparation of Ligand (Ligand 4 as an Example)

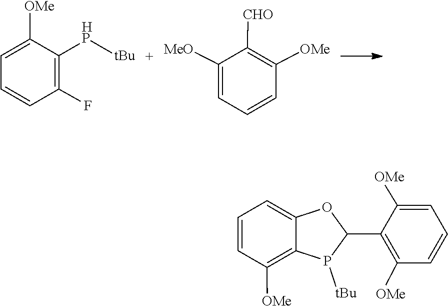

The corresponding substrate phosphine hydrogen (0.2 mmol) was dissolved in 2 mL of dry tetrahydrofuran under an argon atmosphere at room temperature. Then, potassium tert butoxide solid (0.3 mmol, 1.5 equiv.) and the corresponding 2,6-dimethoxybenzaldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the product 4 was obtained with a yield of 88% and a non enantioselectivity greater than 20:1 as a colorless oily fluid.

### Structural Characterization Data of Product 4:

63.4 mg, 80% yield, 24 h, colorless oil. ¹H NMR (400 MHz, Chloroform-d) δ 7.23-7.14 (m, 2H), 6.57-6.40 (m, 5H), 3.85 (s, 3H), 3.64 (s, 6H), 1.07 (d, J=11.9 Hz, 9H). ³¹P NMR (162 MHz, Chloroform-d) δ 13.31. ¹³C NMR (101 MHz, Chloroform-d) δ 166.2, 161.0 (d, J=11.7 Hz), 158.7, 131.3, 129.4 (d, J=1.8 Hz), 117.9 (d, J=16.0 Hz), 112.2 (d, J=14.5 Hz), 104.4, 103.7, 101.8 (d, J=2.4 Hz), 75.7 (d, J=23.9 Hz), 55.8, 55.4, 31.8 (d, J=19.4 Hz), 27.3 (d, J=14.2 Hz).

### Example 16: Preparation of Ligand (Ligand 1 as an Example)

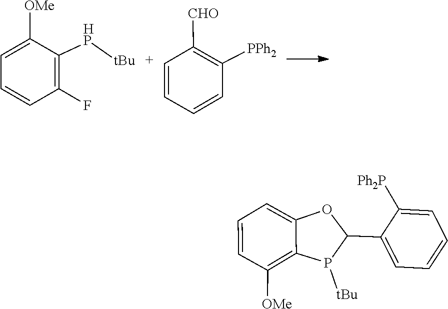

The corresponding substrate phosphine hydrogen (0.2 mmol) was dissolved in 2 mL of dry tetrahydrofuran under an argon atmosphere at room temperature. Then, potassium tert butoxide solid (0.3 mmol, 1.5 equiv.) and the corresponding 2-triphenylphospho benzaldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the product 1 was obtained with a yield of 83% and a non enantioselectivity greater than 20:1 as a white solid.

### Structural Characterization Data of Product 1:

White solid, 80 mg, 83% yield, >20:1 dr. ¹H NMR (600 MHz, CDCl₃) δ (ppm) 7.35-7.31 (m, 6H), 7.31-7.23 (m, 6H), 7.20-7.13 (m, 2H), 7.10 (t, J=7.3 Hz, 1H), 6.90 (dd, J=7.5, 4.8 Hz, 1H), 6.74 (d, J=4.5 Hz, 1H), 6.65 (d, J=8.1 Hz, 1H), 6.49 (dd, J=8.1, 3.5 Hz, 1H), 3.81 (s, 3H), 0.96 (d, J=12.0 Hz, 9H). ¹³C NMR (101 MHz, CDCl₃) δ (ppm) 166.1, 162.4 (d, J=11.7 Hz), 146.5 (dd, J=22.9, 15.4 Hz), 136.6 (dd, J=67.4, 10.5 Hz), 134.7, 134.1 (d, J=18.9 Hz), 133.9 (d, J=19.4 Hz), 132.6, 129.7, 128.8 (d, J=9.8 Hz), 128.7 (d, J=4.8 Hz), 128.6 (d, J=6.6 Hz), 127.7 (d, J=2.1 Hz), 125.6 (t, J=5.7 Hz), 104.1, 103.1 (d, J=2.0 Hz), 83.0 (t, J=28.5 Hz), 55.5, 33.7 (d, J=22.6 Hz), 27.6 (dd, J=13.3, 2.8 Hz). ³¹P NMR (243 MHz, CDCl₃) δ (ppm) 11.07 (d, J=17.7 Hz), −19.44 (d, J=17.6 Hz).

### Example 17: Preparation of Ligand (Ligand 5 as an Example)

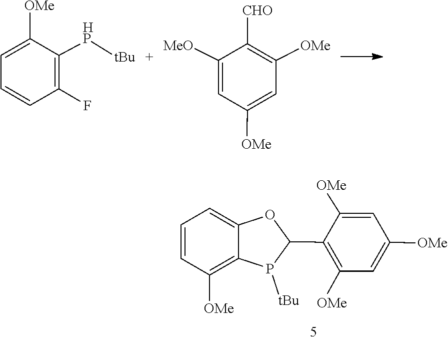

The corresponding substrate phosphine hydrogen (4.0 mmol) was dissolved in 40 mL of dry tetrahydrofuran under an argon atmosphere at room temperature. Then, potassium tert butoxide solid (6.0 mmol, 1.5 equiv.) and the corresponding 2,4,6-trimethoxybenzaldehyde (6.0 mmol, 1.5 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the product 5 was obtained with a yield of 89% and a non enantioselectivity greater than 20:1 as a white solid.

### Structural Characterization Data of Product 5:

White solid, 1.38 g, 89% yield, >20:1 dr. ¹H NMR (400 MHz, CDCl₃) δ (ppm) 7.17 (t, J=8.1 Hz, 1H), 6.50-6.39 (m, 3H), 6.08 (s, 2H), 3.85 (s, 3H), 3.78 (s, 3H), 3.63 (s, 6H), 1.05 (d, J=11.8 Hz, 9H). ¹³C NMR (101 MHz, CDCl₃) δ (ppm) 166.1, 161.3 (d, J=1.5 Hz), 161.0 (d, J=11.5 Hz), 159.5, 131.2, 112.9-110.1 (m), 103.8, 101.7 (d, J=2.2 Hz), 91.1, 75.6 (d, J=23.2 Hz), 55.8, 55.5, 31.8 (d, J=19.2 Hz), 27.3 (d, J=14.2 Hz). ³¹P NMR (162 MHz, CDCl₃) δ (ppm) 12.42.

### Example 18: Preparation of Ligand (Ligand 51 as an Example)

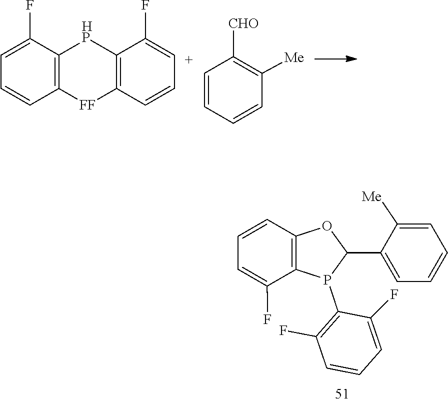

The corresponding substrate phosphine hydrogen (0.2 mmol) was dissolved in 2 mL of dry tetrahydrofuran under an argon atmosphere at room temperature. Then, potassium tert butoxide solid (0.3 mmol, 1.5 equiv.) and the corresponding 2-methylbenzaldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the product 51 was obtained with a yield of 88% and a non enantioselectivity greater than 20:1 as a colorless oily fluid.

### Structural Characterization Data of Product 51:

Colorless oil, 63 mg, 88% yield, >20:1 dr. ¹H NMR (600 MHz, CDCl₃) δ (ppm) 7.40-7.33 (m, 2H), 7.23-7.18 (dd, J=7.2, 2.1 Hz, 3H), 7.17-7.12 (t, J=7.0 Hz, 1H), 6.95 (d, J=8.2 Hz, 1H), 6.90 (t, J=8.1 Hz, 2H), 6.68 (td, J=8.0, 3.8 Hz, 1H), 6.58 (s, 1H), 2.38 (s, 3H). ¹³C NMR (151 MHz, CDCl₃) δ (ppm) 167.0 (d, J=7.6 Hz), 164.8 (dt, J=249.6, 8.7 Hz), 164.5 (dd, J=249.4, 13.6 Hz), 138.0 (d, J=21.0 Hz), 134.3 (d, J=4.8 Hz), 133.8 (d, J=9.2 Hz), 132.9 (t, J=10.7 Hz), 131.0 (d, J=2.2 Hz), 127.9 (d, J=3.3 Hz), 126.4 (d, J=2.5 Hz), 124.4 (d, J=6.2 Hz), 112.7-111.5 (m), 108.2 (d, J=21.8 Hz), 107.1 (d, J=3.4 Hz), 85.7 (d, J=22.3 Hz), 20.0 (d, J=6.3 Hz). ¹⁹F NMR (565 MHz, CDCl₃) δ (ppm) −101.44 (m), −103.10 (m). ³¹P NMR (243 MHz, CDCl₃) δ (ppm) −37.48 (td, J=33.0, 32.4, 6.9 Hz).

### Example 19: Preparation of Ligand (Ligand 52 as an Example)

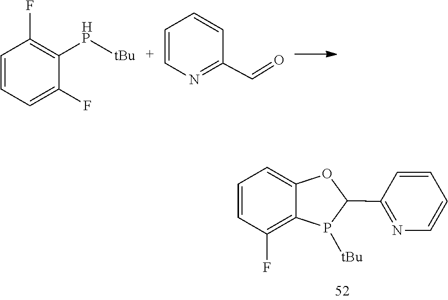

The corresponding substrate phosphine hydrogen (0.2 mmol) was dissolved in 2 mL of dry tetrahydrofuran under an argon atmosphere at room temperature. Then, potassium tert butoxide solid (0.3 mmol, 1.5 equiv.) and the corresponding pyridine-2-aldehyde (0.3 mmol, 1.5 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the product 52 was obtained with a yield of 88% and a non enantioselectivity greater than 20:1 as a white solid.

### Structural Characterization Data of Product 52:

Colorless oil, 51 mg, 88% yield, >20:1 dr. ¹H NMR (400 MHz, CDCl₃) δ (ppm) 8.59 (d, J=4.9 Hz, 1H), 7.58 (td, J=7.7, 1.8 Hz, 1H), 7.32 (td, J=8.1, 6.1 Hz, 1H), 7.20-7.09 (m, 2H), 6.91 (dd, J=8.2, 0.8 Hz, 1H), 6.70 (tdd, J=8.0, 3.2, 0.8 Hz, 1H), 6.09 (s, 1H), 1.14 (d, J=12.7 Hz, 9H). ¹³C NMR (101 MHz, CDCl₃) δ (ppm) 166.0-165.7 (m), 163.4 (d, J=12.6 Hz), 160.6 (d, J=14.3 Hz), 149.7, 136.9, 132.9 (d, J=9.2 Hz), 122.2 (d, J=2.5 Hz), 119.3 (d, J=3.6 Hz), 109.7 (d, J=27.7 Hz), 108.8-108.2 (m), 107.1 (d, J=3.3 Hz), 86.3 (d, J=28.6 Hz), 32.3 (d, J=21.4 Hz), 27.0 (d, J=14.5 Hz). ¹⁹F NMR (376 MHz, CDCl₃) δ (ppm) −102.71. ³¹P NMR (162 MHz, CDCl₃) δ (ppm) 11.69.

### Example 20: Preparation of Ligand (Ligand 53 as an Example)

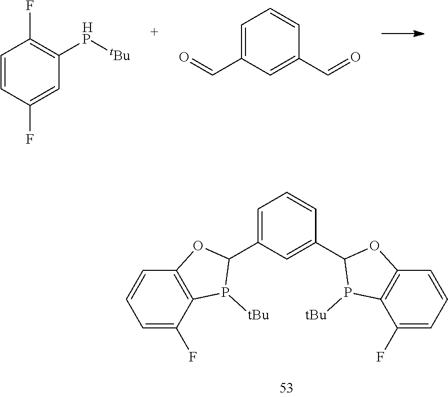

The corresponding substrate phosphine hydrogen (0.5 mmol) was dissolved in 2 mL of dry tetrahydrofuran under an argon atmosphere at room temperature. Then, potassium tert butoxide solid (0.6 mmol, 3.0 equiv.) and the corresponding 1,3-benzenedialdehyde (0.2 mmol, 1.0 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the product 53 was obtained with a yield of 49% and a non enantioselectivity of 6:1 as a white solid.

### Structural Characterization Data of Product 53:

White solid, 49 mg, 49% yield, 6:1 dr. ¹H NMR (400 MHz, CDCl₃) δ (ppm) 7.31-7.19 (m, 3H), 7.10 (d, J=6.7 Hz, 3H), 6.79 (ddd, J=13.7, 8.1, 0.8 Hz, 2H), 6.67 (dt, J=8.0, 4.1 Hz, 2H), 5.94 (d, J=3.4 Hz, 2H), 1.08 (d, J=12.6 Hz, 18H). ¹³C NMR (101 MHz, CDCl₃) δ (ppm) 165.8 (d, J=8.5 Hz), 163.4 (d, J=12.4 Hz), 141.7 (d, J=16.6 Hz), 132.9 (d, J=9.2 Hz), 129.0, 124.0 (dt, J=7.8, 2.1 Hz), 121.5 (t, J=7.0 Hz), 109.4 (dd, J=27.6, 20.8 Hz), 108.2 (d, J=22.7 Hz), 107.1-106.6 (m), 84.9 (d, J=28.1 Hz), 32.1 (d, J=20.8 Hz), 27.0 (d, J=14.6 Hz). ¹⁹F NMR (376 MHz, CDCl₃) δ (ppm) −102.99. ³¹P NMR (162 MHz, CDCl₃) δ (ppm) 12.36, 12.30. HRMS (ESI-TOF) m/z: [M+H]⁺ Calcd for C₂₈H₃₁F₂O₂P₂⁺=499.1762; Found 499.1761.

### Example 21: Preparation of Ligand (Ligand 54 as an Example)

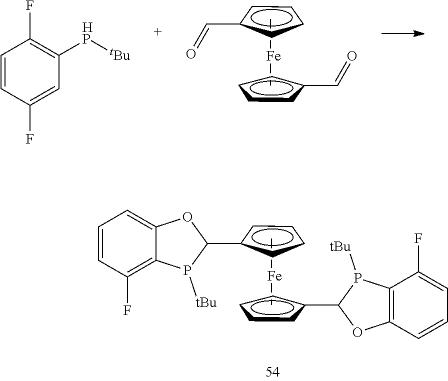

The corresponding substrate phosphine hydrogen (0.5 mmol) was dissolved in 2 mL of dry tetrahydrofuran under an argon atmosphere at room temperature. Then, potassium tert butoxide solid (0.6 mmol, 3.0 equiv.) and the corresponding ferrocenedicarboxaldehyde (0.2 mmol, 1.0 equiv.) were added in sequence, and the mixture was kept stirring until the raw materials were completely consumed detected by TLC. Purification was carried out by column chromatography, with degassed petroleum ether ethyl acetate (100:0-20:1) as the eluent, and the product 54 was obtained with a yield of 49% and a non enantioselectivity greater than 20:1 as a yellow liquid.

### Structural Characterization Data of Product 54:

Yellow oil, 53 mg, 44% yield, >20:1 dr. ¹H NMR (400 MHz, CDCl₃) δ (ppm) 7.24 (td, J=8.1, 6.2 Hz, 2H), 6.74 (t, J=7.8 Hz, 2H), 6.65 (tdd, J=7.9, 3.4, 1.5 Hz, 2H), 5.75 (d, J=7.5 Hz, 2H), 4.24-3.97 (m, 8H), 1.07 (dd, J=12.6, 4.3 Hz, 18H). ¹³C NMR (101 MHz, CDCl₃) δ (ppm) 165.4 (dd, J=8.8, 5.3 Hz), 164.4 (ddd, J=246.7, 12.4, 3.8 Hz), 132.8 (d, J=9.1 Hz), 110.3 (ddd, J=27.4, 19.0, 4.7 Hz), 107.7 (d, J=22.9 Hz), 107.3 (dd, J=5.2, 3.2 Hz), 89.1 (dd, J=21.9, 15.5 Hz), 82.2 (dd, J=26.6, 6.1 Hz), 69.4, 69.4 (d, J=30.5 Hz), 68.4 (dd, J=46.1, 8.2 Hz), 67.5 (dd, J=14.7, 8.0 Hz), 32.0 (d, J=19.3 Hz), 27.1 (dd, J=14.5, 2.6 Hz). ¹⁹F NMR (376 MHz, CDCl₃) δ (ppm) −103.46. ³¹P NMR (162 MHz, CDCl₃) δ (ppm) 1.63, 1.08.

### Example 22: Preparation of Ligand (Ligand 55 as an Example)

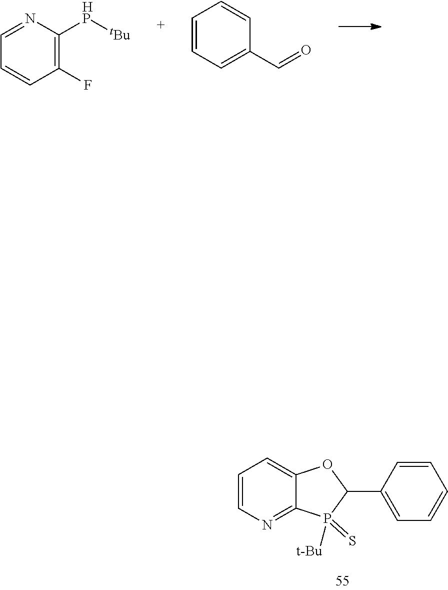

The corresponding substrate phosphine hydrogen (0.2 mmol) was dissolved in 2 mL of dry tetrahydrofuran under an argon atmosphere at room temperature. Then, potassium tert butoxide solid (0.3 mmol, 1.5 equiv.) and the corresponding bromobenzaldehyde (0.3 mmol, 1.5 equiv.) were added in sequence, and the mixture was kept stirring until the raw materials were completely consumed detected by TLC. Subsequently, sulfur powder (0.6 mmol, 3.0 equiv.) was added under an argon atmosphere and stirred at room temperature for 0.5 hours. Purification was carried out by column chromatography, with degassed petroleum ether ethyl acetate (100:0-20:1) as the eluent, and the product 55 was obtained with a yield of 91% and a non enantioselectivity greater than 20:1 as a white solid.

### Structural Characterization Data of Product 55:

White solid, 55 mg, 91% yield, >20:1 dr. ¹H NMR (400 MHz, CDCl₃) δ (ppm) 8.49 (dd, J=3.9, 1.8 Hz, 1H), 7.41-7.32 (m, 5H), 7.25-7.22 (m, 2H), 5.93 (d, J=1.4 Hz, 1H), 1.36 (d, J=17.6 Hz, 9H). ¹³C NMR (101 MHz, CDCl₃) δ (ppm) 158.0 (d, J=28.7 Hz), 146.2 (d, J=14.9 Hz), 140.4 (d, J=101.5 Hz), 133.8 (d, J=2.2 Hz), 128.6 (d, J=2.6 Hz), 128.1 (d, J=1.8 Hz), 127.1 (d, J=2.8 Hz), 126.9 (d, J=4.3 Hz), 120.0 (d, J=5.0 Hz), 79.2 (d, J=40.4 Hz), 36.2 (d, J=47.9 Hz), 24.4. ³¹P NMR (162 MHz, CDCl₃) δ (ppm) 65.47.

### Example 23: Preparation of Ligand (Ligand 21 as an Example)

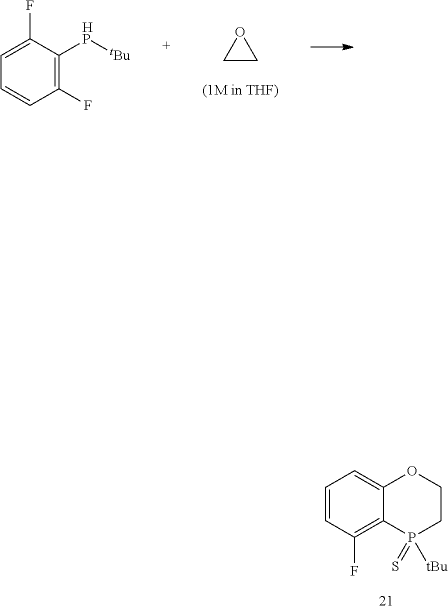

The corresponding substrate phosphine hydrogen (0.2 mmol) was dissolved in 1 mL of dry tetrahydrofuran under an argon atmosphere at room temperature. Then, potassium tert butoxide solid (0.3 mmol, 1.5 equiv.) and the corresponding ethylene oxide (1.0 mL, 1.0 mmol, 5.0 equiv.) were added in sequence, and the mixture was kept stirring until the raw materials were completely consumed detected by TLC. Subsequently, sulfur powder (0.6 mmol, 3.0 equiv.) was added under an argon atmosphere and stirred at room temperature for 0.5 hours. Purification was carried out by column chromatography, with degassed petroleum ether ethyl acetate (100:0-20:1) as the eluent, and the product 21 was obtained with a yield of 80% as a colorless oily fluid.

### Structural Characterization Data of Product 21:

82.0 mg, 80% yield, 24 h, colorless oil. ¹H NMR (400 MHz, CDCl₃) δ (ppm) 7.33 (q, J=7.9 Hz, 1H), 6.79-6.70 (m, 2H), 4.66-4.52 (m, 1H), 4.51-4.41 (m, 1H), 2.85-2.75 (m, 1H), 2.22-2.08 (m, 1H), 1.27 (d, J=18.1 Hz, 9H). ¹³C NMR (100 MHz, CDCl₃) δ (ppm) 164.1 (d, J=250.1 Hz), 160.5 (dd, J=7.3, 1.4 Hz), 133.5 (d, J=10.7 Hz), 114.8 (dd, J=4.4, 3.6 Hz), 109.1 (dd, J=23.2, 4.4 Hz), 105.4 (dd, J=63.9, 22.8 Hz), 77.3, 77.0, 76.7, 63.4 (d, J=6.6 Hz), 35.8 (d, J=52.1 Hz), 30.1 (d, J=51.7 Hz), 25.3 (dd, J=4.3, 2.8 Hz). ¹⁹F NMR (376 MHz, CDCl₃) δ (ppm) −96.10. ³¹P NMR (162 MHz, CDCl₃) δ (ppm) 39.40.

### Example 24: Preparation of Ligand (Ligand 23 as an Example)

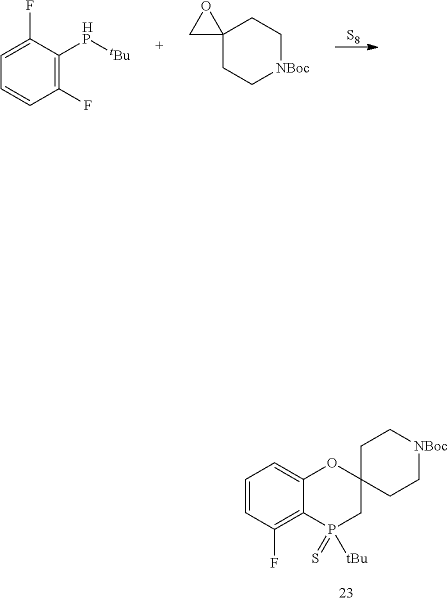

The corresponding substrate phosphine hydrogen (0.4 mmol) was dissolved in 1 mL of dry tetrahydrofuran under an argon atmosphere at room temperature. Then, potassium tert butoxide solid (0.6 mmol, 1.5 equiv.) and the corresponding epoxy compound (0.6 mmol, 1.5 equiv.) were added in sequence, and the mixture was kept stirring until the raw materials were completely consumed detected by TLC. Subsequently, sulfur powder (4.0 mmol, 10.0 equiv.) was added under an argon atmosphere and stirred at room temperature for 2 hours. Purification was carried out by column chromatography, with degassed petroleum ether ethyl acetate (100:0-5:1) as the eluent, and the product 23 was obtained with a yield of 89% as a white solid.

### Structural Characterization Data of Product 23:

White solid, 152 mg, 89% yield. ¹H NMR (400 MHz, CDCl₃) δ (ppm) 7.38 (q, J=8.1 Hz, 1H), 6.84-6.77 (m, 2H), 4.04-3.84 (m, 1H), 3.80-3.66 (m, 1H), 3.40 (t, J=10.5 Hz, 1H), 2.95-2.82 (m, 1H), 2.70 (dd, J=14.5, 6.0 Hz, 1H), 2.44 (d, J=15.0 Hz, 1H), 2.22 (t, J=14.9 Hz, 1H), 1.91-1.76 (m, 3H), 1.45 (s, 9H), 1.22 (d, J=18.0 Hz, 9H). ¹³C NMR (101 MHz, CDCl₃) δ (ppm) 164.2 (d, J=253.0 Hz), 157.0 (d, J=6.3 Hz), 154.7, 133.9 (d, J=11.2 Hz), 115.6 (dd, J=4.5, 3.2 Hz), 110.0 (dd, J=23.5, 4.6 Hz), 105.4 (dd, J=64.3, 20.4 Hz), 79.8, 75.7 (d, J=5.8 Hz), 39.8, 39.3, 39.1, 39.0, 36.9, 36.4, 31.8, 28.4, 24.7 (t, J=2.9 Hz). ¹⁹F NMR (376 MHz, CDCl₃) δ (ppm) −95.21. ³¹P NMR (162 MHz, CDCl₃) δ (ppm) 32.17.

### Example 25: Palladium Catalyzed Suzuki Coupling Reaction

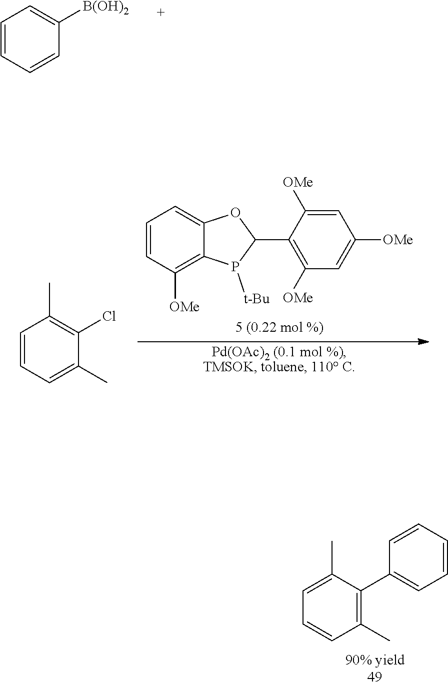

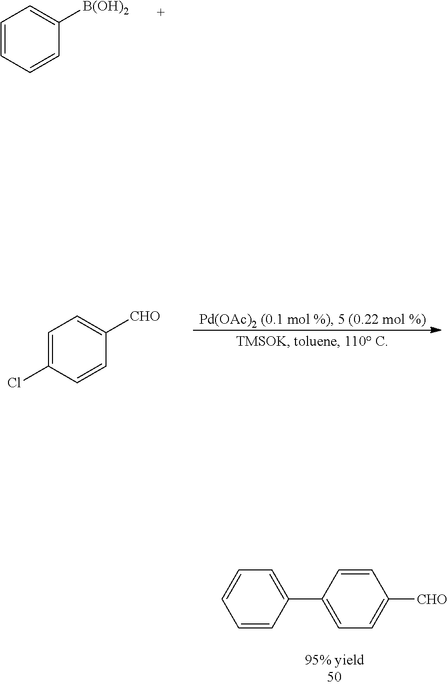 [SMILES: OB(O)c1ccccc1 ; O=Cc1ccc(Cl)cc1 ; "Pd(OAc)2 (0.1 mol %), 5 (0.22 mol %)" ; "TMSOK, toluene, 110° C." ; O=Cc1ccc(-c2ccccc2)cc1]

Under an argon atmosphere at room temperature, corresponding aryl chlorides (0.5 mmol), phenylboronic acid (0.75 mmol), and potassium trimethylsilanol (1.0 mmol) were sequentially added to a 10 mL pressure resistant sealed tube, and the mixture was dissolved in 1 mL of toluene, and finally a pre-complexed palladium/ligand complex solution (0.1% mol) was added. The reaction was carried out at 100° C. for 24 hours. Purification was carried out by column chromatography to obtain the final product 49 or 50.

### Structural Characterization Data of Product 49:

82.0 mg, 90% yield, 24 h, colorless oil. ¹H NMR (400 MHz, CDCl₃): δ (ppm) 7.16-7.24 (m, 5H), 7.39 (dt, J=7.0, 1.2 Hz, 1H), 7.48 (dt, J=7.0, 1.1 Hz, 2H), 2.10 (s, 6H). ¹³C NMR (100 MHz, CDCl₃): δ (ppm) 141.8, 141.0, 136.0, 129.0, 128.4, 127.2, 127.0, 126.6, 20.8.

### Structural Characterization Data of Product 50:

86.5 mg, 95% yield, 24 h, colorless oil. ¹H NMR (400 MHz, DMSO-d₆) δ 10.04 (s, 1H), 8.05-7.94 (m, 2H), 7.93-7.85 (m, 2H), 7.83-7.71 (m, 2H), 7.54-7.39 (m, 3H). ¹³C NMR (101 MHz, DMSO-d₆) δ (ppm) 193.2, 146.3, 139.3, 135.6, 130.6, 129.6, 129.1, 127.8, 127.6.

### Example 26: Palladium Catalyzed C—N Coupling Reaction

Ligand 56 was prepared by using the method described in Example 3.

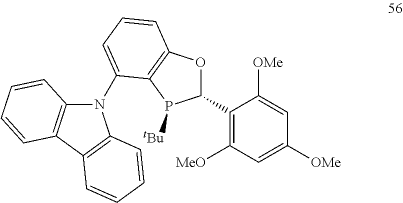 [SMILES: COc1cc(OC)c([C@H]2Oc3cccc(-n4c5ccccc5c5ccccc54)c3[P@]2C(C)(C)C)c(OC)c1]

### Structural Characterization Data of Product 56:

White solid. ¹H NMR (400 MHz, CDCl₃): δ (ppm) 8.10 (dd, J=7.7, 3.8 Hz, 2H), 7.56 (dd, J=8.2, 2.9 Hz, 1H), 7.40 (ddd, J=12.3, 8.7, 4.9 Hz, 4H), 7.30-7.21 (m, 2H), 7.00 (dd, J=7.7, 2.9 Hz, 1H), 6.95 (d, J=8.1 Hz, 1H), 6.53 (d, J=3.8 Hz, 1H), 6.17 (s, 2H), 3.82 (s, 3H), 3.74 (s, 6H), 0.59 (d, J=12.1 Hz, 9H). ¹³C NMR (100 MHz, CDCl₃): δ (ppm) 166.7, 161.3, 159.1, 141.4, 139.1, 139.0, 138.9, 131.2, 125.8, 125.3, 124.2, 123.9, 123.6, 123.2, 120.2, 119.9, 119.8, 119.7, 119.5, 119.4, 112.1, 112.0, 110.4, 110.3, 110.1, 109.4, 91.0, 77.3, 77.0, 76.7, 75.5, 75.2, 55.7, 55.4, 31.4, 31.2, 26.5, 26.3. ³¹P NMR (162 MHz, CDCl₃) δ 15.52.

### Preparation of Compound 57:

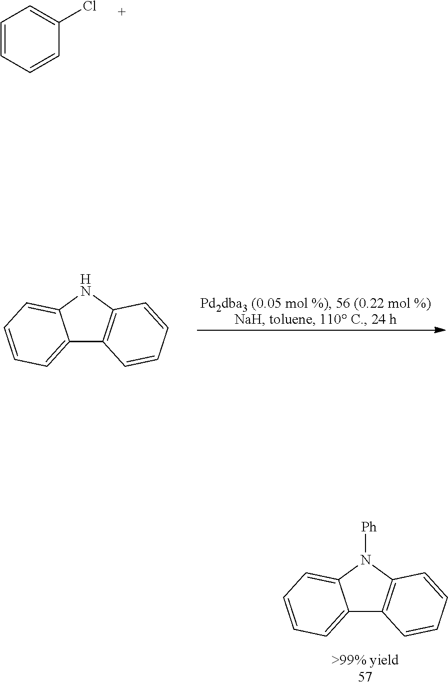 [SMILES: Clc1ccccc1 ; c1ccc2c(c1)[nH]c1ccccc12 ; "Pd2dba3 (0.05 mol %), 56 (0.22 mol %)NaH, toluene, 110° C., 24 h" ; c1ccc(-n2c3ccccc3c3ccccc32)cc1]

Under an argon atmosphere at room temperature, a toluene solution with a molar concentration of 0.002 mmol/mL of [Pd] was prepared from the corresponding Pd₂dba₃ and ligand 56 in a 0.5:2.2 molar ratio (e.g. Pd₂dba₃ (0.05 mol %), 56 (0.22 mol %)). The complex solution was stirred for 30 minutes under an argon atmosphere. Then, the corresponding carbazole (1.1 mmol) and sodium hydride (1.1 mmol) were added to a dry 10 mL pressure resistant sealed tube, and 1 mL of toluene was added. The mixture was stirred at room temperature for 10 minutes, followed by the addition of chlorobenzene (1.0 mmol), the corresponding [Pd] catalyst, and further toluene was added to make the solvent volume of the reaction 3.0 mL. After the addition was complete, the reaction tube was placed at 110° C. for 24 hours. Subsequently, the solvent was removed by vacuum distillation, and the final target product 57 was obtained by column chromatography using pure petroleum ether as the eluent. The corresponding separation yield was described in the above equation.

When Pd₂dba₃ is 0.025 mol % and 56 is 0.11 mol %, the reaction can also proceed smoothly.

### Structural Characterization Data of Product 57:

White solid. ¹H NMR (400 MHz, CDCl₃): δ (ppm) 8.09 (d, J=7.8 Hz, 2H), 7.53-7.46 (m, 4H), 7.41-7.31 (m, 5H), 7.23 (ddd, J=8.0, 5.8, 2.3 Hz, 2H). ¹³C NMR (400 MHz, CDCl₃) δ 140.8, 137.6, 129.8, 127.3, 127.0, 125.9, 123.3, 120.2, 119.9, 109.7, 77.3, 77.0, 76.7.

### Preparation of Compound 58:

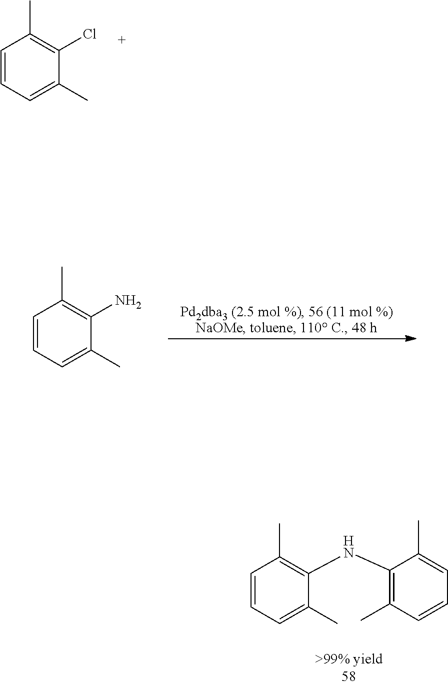

The corresponding Pd₂dba₃ (4.6 mg, 0.005 mmol) and ligand 56 (11.55 mg, 0.022 mmol) were dissolved in 1 mL of toluene under an argon atmosphere at room temperature. The solution of the complex was stirred under an argon atmosphere for 30 minutes. Then, the corresponding 2,6-dimethylchlorobenzene (0.2 mmol), 2,6-dimethylaniline (0.4 mmol) and sodium methoxide (0.4 mmol) were added to a dry 10 mL pressure sealed tube, and 1 mL of toluene was added. After the addition was complete, the reaction tube was placed at 110° C. for 24 hours. Subsequently, the solvent was removed by vacuum distillation, and the final target product 58 was obtained by column chromatography using pure petroleum ether as the eluent. The corresponding separation yield was described in the above equation.

### Structural Characterization Data of Product 58:

White solid, 45.1 mg, >99% yield. ¹H NMR (400 MHz, CDCl₃): δ (ppm) 6.97 (d, J=7.5 Hz, 4H), 6.83 (t, J=7.5 Hz, 2H), 4.79 (s, 1H), 2.00 (s, 12H). ¹³C NMR (400 MHz, CDCl₃) δ 13C NMR (101 MHz, CDCl₃) δ 141.7, 129.5, 128.7, 121.7, 77.3, 77.0, 76.7, 19.1.

### Example 27

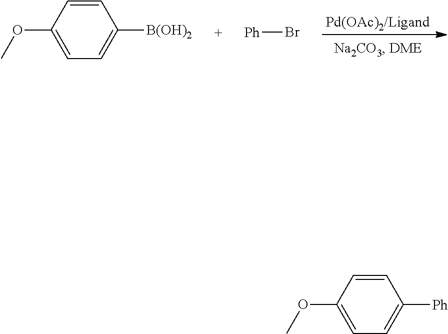

Under an argon atmosphere, bromobenzene (3.14 g, 20 mmol), p-methoxyphenylboronic acid (3.65 g, 24 mmol), palladium acetate (44.8 mg, 0.2 mmol), ligand I-15 prepared in the present embodiment (131.4 mg, 0.4 mmol) were added to a 100 mL flask. Then, sodium carbonate solution (3.0 M, 20 mL) and 20 mL of dimethoxyethane were added. The reaction system was heated to reflux for 24 hours, and 4-phenylbenzyl ether (3.35 g, 91% yield) was obtained by column chromatography, petroleum ether and ethyl acetate were used as developing agents (PE/EA=20/1).

### Example 28

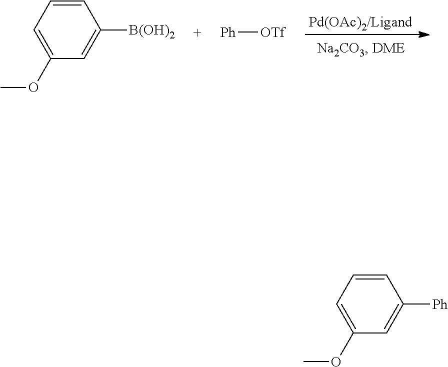

Under an argon atmosphere, PhOTf (4.52 g, 20 mmol), m-methoxyphenylboronic acid (3.65 g, 24 mmol), palladium acetate (44.8 mg, 0.2 mmol), ligand I-15 (131.4 mg, 0.4 mmol) were added to a 100 mL flask. Then, sodium carbonate solution (3.0 M, 20 mL) and 20 mL of dimethoxyethane were added. The reaction system was heated to reflux for 24 hours, and 3-phenylbenzyl ether (3.50 g, 95% yield) was obtained by column chromatography, petroleum ether and ethyl acetate were used as developing agents (PE/EA=20/1).

### Example 29

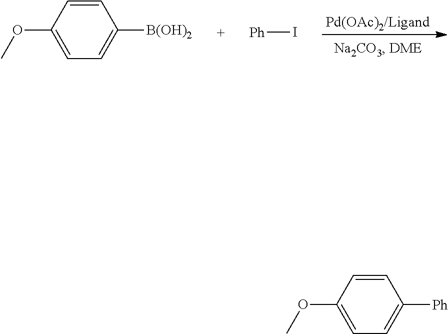

Under an argon atmosphere, iodobenzene (4.08 g, 20 mmol), p-methoxyphenylboronic acid (3.65 g, 24 mmol), palladium acetate (44.8 mg, 0.2 mmol), ligand I-15 (131.4 mg, 0.4 mmol) were added to a 100 mL flask. Then, sodium carbonate solution (3.0 M, 20 mL) and 20 mL of dimethoxyethane were added. The reaction system was heated to reflux for 24 hours, and 4-phenylbenzyl ether (3.46 g, 94% yield) was obtained by column chromatography, petroleum ether and ethyl acetate were used as developing agents (PE/EA=20/1).

The method of the present invention has been described by the preferred examples, and it is apparent that related persons can make modifications or proper changes and combination to the compounds and preparation methods described herein so as to achieve and apply the technology of the present application, without departing from the content, spirit and scope of the present invention. Those skilled in the art may use the content herein for reference to improve the technological parameters appropriately to achieve it. In particularly, it should be pointed out, that all similar substitutions and modifications are obvious to those skilled in the art and are considered to be included in the present invention.

What is claimed is:

1. A substituted 2,3-dihydrobenzo[d][1,3]oxaphosphole ligand, characterised in that it is represented by the structure of formula (I), 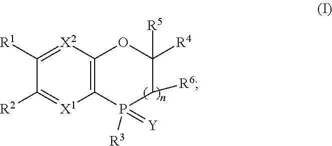
wherein R⁴ is hydrogen, alkyl, alkenyl, cycloalkyl, heterocyclyl, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)—, dicycloalkyloxyphospho(═O)—, ferrocene, aryl, heteroaryl, alkyloxy, cycloalkyloxy, heterocyclyloxy, aryloxy, heteroaryloxy, cycloalkylalkyl, heterocyclylalkyl, arylalkyl or heteroarylalkyl; alternatively, R⁴ and R⁵, together with the carbon atom to which they are attached, form a cycloalkyl or heterocyclyl group; alternatively, R⁴ and the adjacent R⁶, together with the carbon atoms to which they are attached, form a cycloalkyl or heterocyclyl group;
X¹ is N or CR^{X1};
X² is N or CR^{X2};
each of R^{X1} and R^{X2} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy, —SO₂NMe₂ or halo; 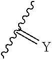
is ═O or ═S; or 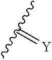
is absent;
R¹ is hydrogen, alkyl, aryl, heteroaryl, alkyloxy, —SO₂NMe₂ or halo;
R² is hydrogen, alkyl, aryl, heteroaryl, alkyloxy, —SO₂NMe₂ or halo;
or R¹ and R², together with the carbon atoms to which they are attached, form an aryl or heteroaryl group;
R³ is hydrogen, alkyl, cycloalkyl, aryl, heteroaryl or alkyloxy;
R⁵ is hydrogen, alkyl, aryl, heteroaryl or alkyloxy;
R⁶ is hydrogen;
n is 0 or 1;
or R⁴ is 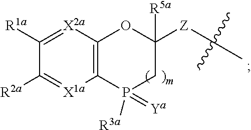
wherein X^{1a} is N or CRX^{1a}, X^{2a} is N or CR^{X2a};
each of R^{X1a} and R^{X2a} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy, fluoro, chloro, bromo or iodo; 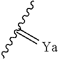
is ═O or ═S; or 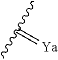
is absent;
Z is a bond, cycloalkylene, heterocyclylene, arylene, heteroarylene, alkylene or 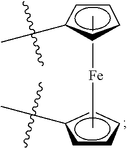
R^{1a} is hydrogen, alkyl, aryl, heteroaryl, alkyloxy or halo;
R^{2a} is hydrogen, alkyl, aryl, heteroaryl, alkyloxy or halo;
or R^{1a} and R^{2a}, together with the carbon atoms to which they are attached, form an aryl or heteroaryl group;
R^{3a} is hydrogen, alkyl, cycloalkyl, aryl, heteroaryl or alkyloxy;
R^{5a} is hydrogen, alkyl, aryl, heteroaryl or alkyloxy;
m is 0 or 1;
the alkyl, alkenyl, cycloalkyl, heterocyclyl, aryl, heteroaryl, cycloalkylene, heterocyclylene, arylene, heteroarylene and alkylene mentioned in the single groups or complex groups hereinabove are each independently optionally substituted with 1, 2, 3, 4 or 5 substituents selected from hydroxy, amino, halo, carboxyl, sulfonyl, alkyl, haloalkyl, cycloalkyl, heterocyclyl, aryl, heteroaryl, alkyloxy, alkylthio, cycloalkyloxy, heterocyclyloxy, aryloxy, heteroaryloxy, alkyl-NH—, (alkyl)₂N—, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O) and dicycloalkyloxyphospho(═O)—.

2. The substituted 2,3-dihydrobenzo[d][1,3]oxaphosphole ligand of claim 1, characterised in that it is represented by the structure of formula I-A or II-A, 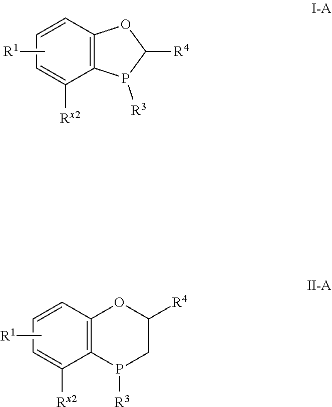
wherein
R¹ is H, F or SO₂NMe₂;
R^{X2} is F, Cl, Br or I;
R³ is aryl or alkyl;
R⁴ is hydrogen, aryl, heteroaryl or alkyl;
the aryl or heteroaryl is unsubstituted or substituted with 1, 2, 3, 4 or 5 substituents selected from halo, —PPh₂, —N(alkyl)₂, C₁₋₆alkyl, C₁₋₆haloalkyl, C₁₋₆alkyloxy and C₁₋₆alkylthio.

3. The substituted 2,3-dihydrobenzo[d][1,3]oxaphosphole ligand of claim 1, characterised in that
R¹ is H or F;
R^{X2} is F;
R³ is C₆₋₁₀aryl or C₁₋₆alkyl;
R⁴ is C₆₋₁₀aryl, 3-6 membered heteroaryl or C₁₋₆alkyl;
the C₆₋₁₀aryl, 3-6 membered heteroaryl or C₁₋₆alkyl is unsubstituted or substituted with 1, 2, 3, 4 or 5 substituents selected from halo, —PPh₂, —NMe₂, SMe₂, —SMe, C₁₋₆haloalkyl and C₁₋₆alkyloxy.

4. The substituted 2,3-dihydrobenzo[d][1,3]oxaphosphole ligand of claim 1, characterised in that it is selected from one of the following structures:

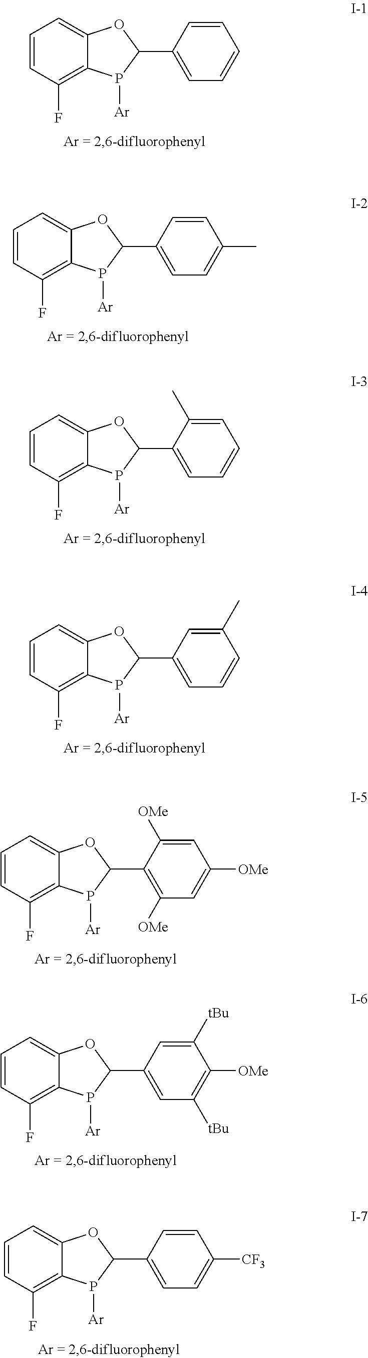

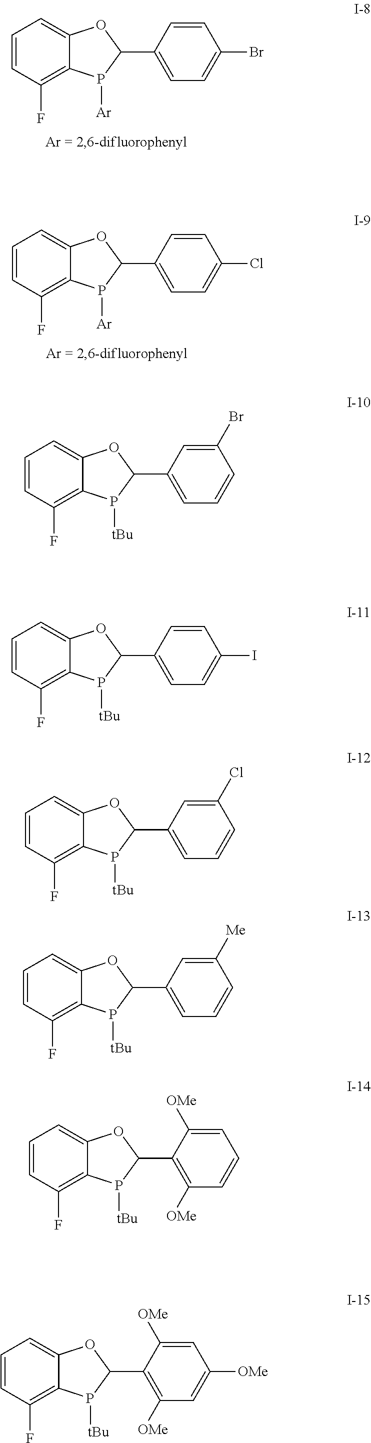

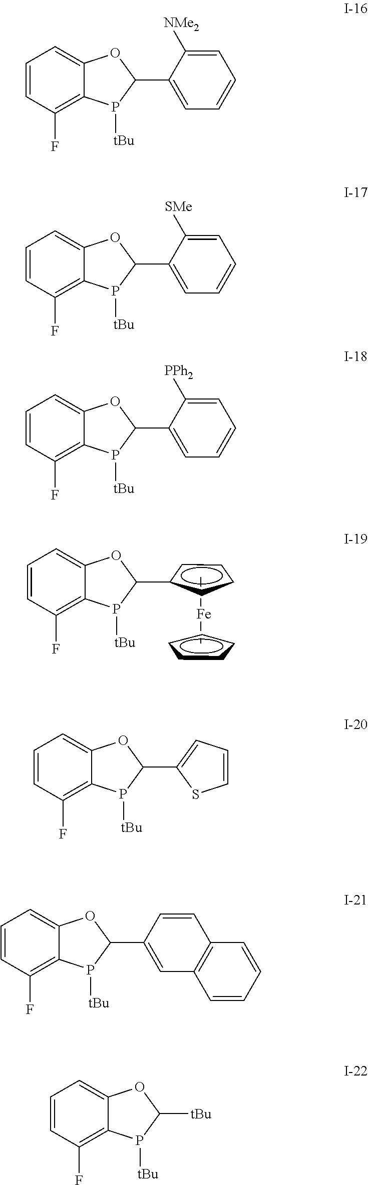

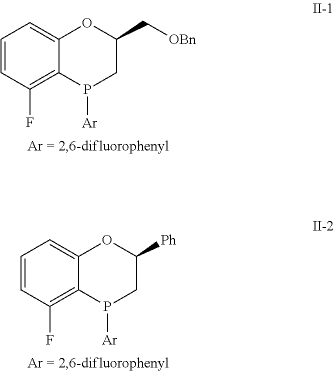

5. The substituted 2,3-dihydrobenzo[d][1,3]oxaphosphole ligand of claim 1, characterised in that it is represented by the structure of formula I-B, 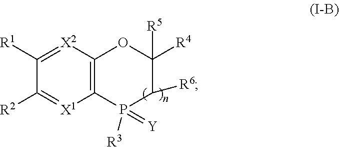
wherein R⁴ is hydrogen, alkyl, alkenyl, cycloalkyl, heterocyclyl, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)—, dicycloalkyloxyphospho(═O)—, ferrocene, aryl, heteroaryl, alkyloxy, cycloalkyloxy, heterocyclyloxy, aryloxy, heteroaryloxy, cycloalkylalkyl, heterocyclylalkyl, arylalkyl or heteroarylalkyl; alternatively, R⁴ and R⁵, together with the carbon atom to which they are attached, form a cycloalkyl or heterocyclyl group; alternatively, R⁴ and the adjacent R⁶, together with the carbon atoms to which they are attached, form a cycloalkyl or heterocyclyl group;
X¹ is N or CR^{X1};
X² is N or CR^{X2};
each of R^{X1} and R^{X2} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy or halo; 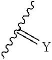
is ═O or ═S; or 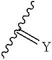
is absent;
R¹ is hydrogen, alkyl, aryl, heteroaryl, alkyloxy or halo;
R² is hydrogen, alkyl, aryl, heteroaryl, alkyloxy or halo;
or R¹ and R², together with the carbon atoms to which they are attached, form an aryl or heteroaryl group;
R³ is hydrogen, alkyl, cycloalkyl, aryl, heteroaryl or alkyloxy;
R⁵ is hydrogen, alkyl, aryl, heteroaryl or alkyloxy;
R⁶ is hydrogen;
n is 0 or 1;
or R⁴ is 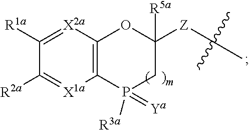
wherein X^{1a} is N or CRX^{1a};
X^{2a} is N or CR^{X2a};
each of R^{X1a} and R^{X2a} is independently hydrogen, amino, hydroxy, alkyl-NH—, (alkyl)₂N—, alkyl, cycloalkyl, heterocyclyl, aryl, heteroaryl, alkyloxy, cycloalkyloxy, heterocyclyloxy, aryloxy, heteroaryloxy, fluoro, chloro, bromo or iodo; 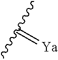
is ═O or ═S; or 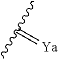
is absent;
Z is a bond, cycloalkylene, heterocyclylene, arylene, heteroarylene, alkylene or 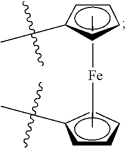
R^{1a} is hydrogen, alkyl, aryl, heteroaryl, alkyloxy or halo;
R^{2a} is hydrogen, alkyl, aryl, heteroaryl, alkyloxy or halo;
or R^{1a} and R^{2a}, together with the carbon atoms to which they are attached, form an aryl or heteroaryl group;
R^{3a} is hydrogen, alkyl, cycloalkyl, aryl, heteroaryl or alkyloxy;
R^{5a} is hydrogen, alkyl, aryl, heteroaryl or alkyloxy;
m is 0 or 1;
the alkyl, alkenyl, cycloalkyl, heterocyclyl, aryl, heteroaryl, cycloalkylene, heterocyclylene, arylene, heteroarylene and alkylene mentioned in the single groups or complex groups hereinabove are each independently optionally substituted with 1, 2, 3, 4 or 5 substituents selected from hydroxy, amino, halo, carboxyl, sulfonyl, alkyl, haloalkyl, cycloalkyl, heterocyclyl, aryl, heteroaryl, alkyloxy, alkylthio, cycloalkyloxy, heterocyclyloxy, aryloxy, heteroaryloxy, alkyl-NH—, (alkyl)₂N—, alkyl-OC(═O)—, alkyl-C(═O)O—, dialkylphospho, diarylphospho, di(3,5-dimethyl-4-methoxyphenyl)phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O)—, dicycloalkylphospho(═O)—, dialkoxyphospho(═O)—, diaryloxyphospho(═O)— and dicycloalkyloxyphospho(═O)—;
with the proviso that the structures of formula (I-B) do not include the following structures: 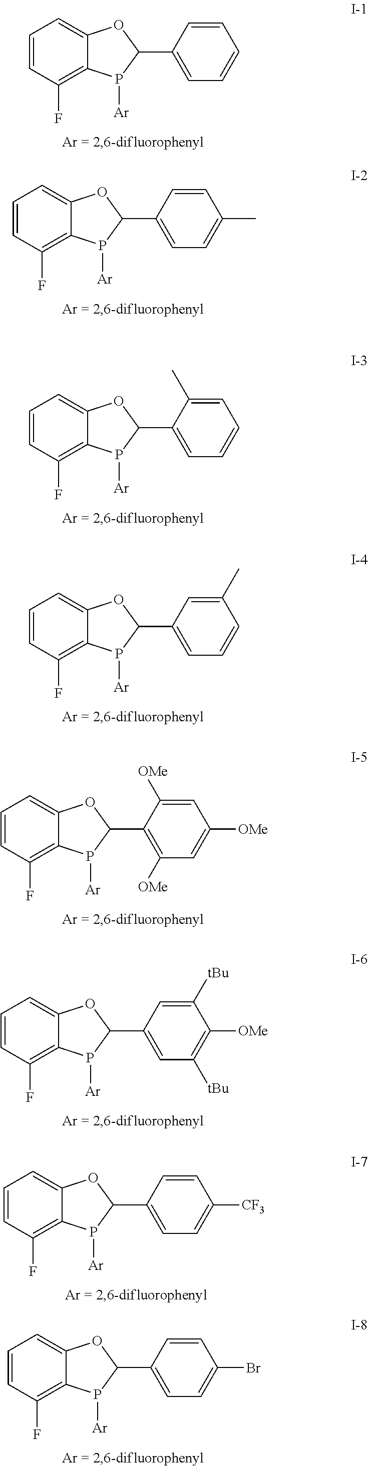 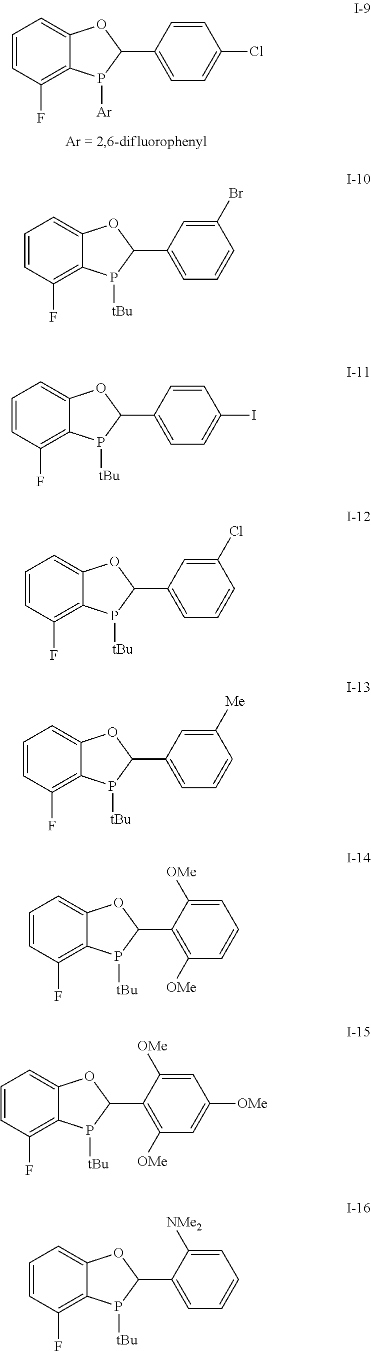 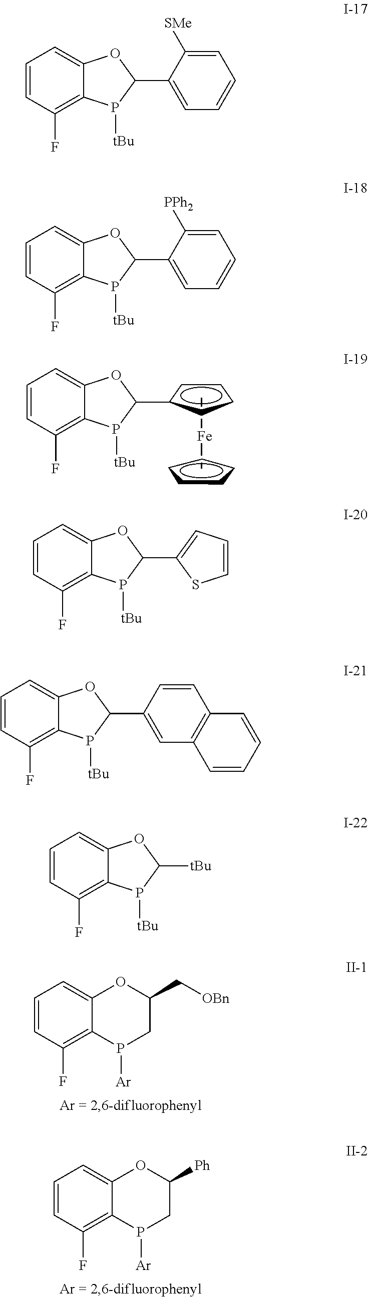
or with the proviso that in the structure of formula (I-B), when R^{X1a} is F, 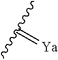
is ═O or ═S;
or with the proviso that in the structure of formula (I-B), when R^{X1a} is F, R⁴ is hydrogen, alkyl, alkenyl, cycloalkyl, heterocyclyl, alkylOC(═O)—, alkylC(═O)O—, dialkylphospho, diarylphospho, di(3,5-dimethyl-4-methoxyphenyl) phospho, di(3,5-di-tert-butyl-4-methoxyphenyl)phospho, dicycloalkylphospho, dialkoxyphospho, diaryloxyphospho, dicycloalkyloxyphospho, dialkylphospho(═O)—, diarylphospho(═O), dicycloalkylphospho(═O)—, dialkoxyphospho(═O)—, diaryloxyphospho(═O), dicycloalkyloxyphospho(═O), ferrocene, alkyloxy, cycloalkyloxy, heterocyclyloxy, aryloxy, heteroaryloxy, cycloalkylalkyl, heterocyclylalkyl, arylalkyl or heteroarylalkyl; alternatively, R⁴ and R⁵, together with the carbon atom to which they are attached, form a cycloalkyl or heterocyclyl group; alternatively, R⁴ and the adjacent R⁶, together with the carbon atoms to which they are attached, form a cycloalkyl or heterocyclyl group; or R⁴ is

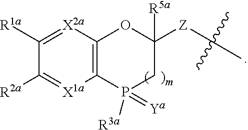

6. The substituted 2,3-dihydrobenzo[d][1,3]oxaphosphole ligand of claim 1, characterised in that each of 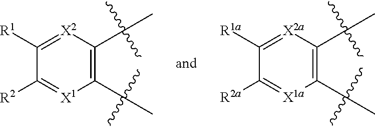
is independently selected from:

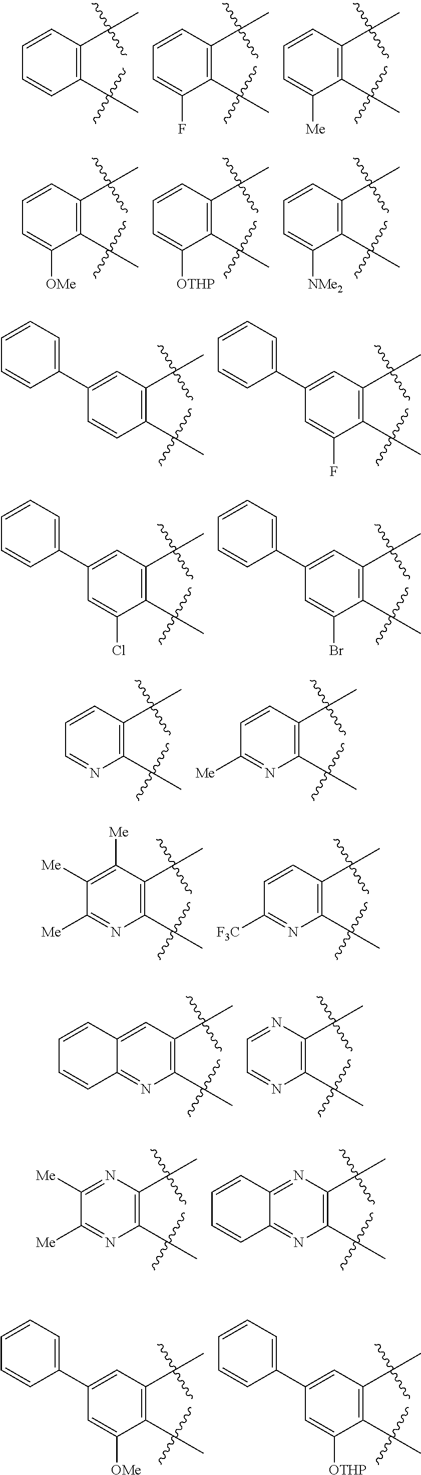

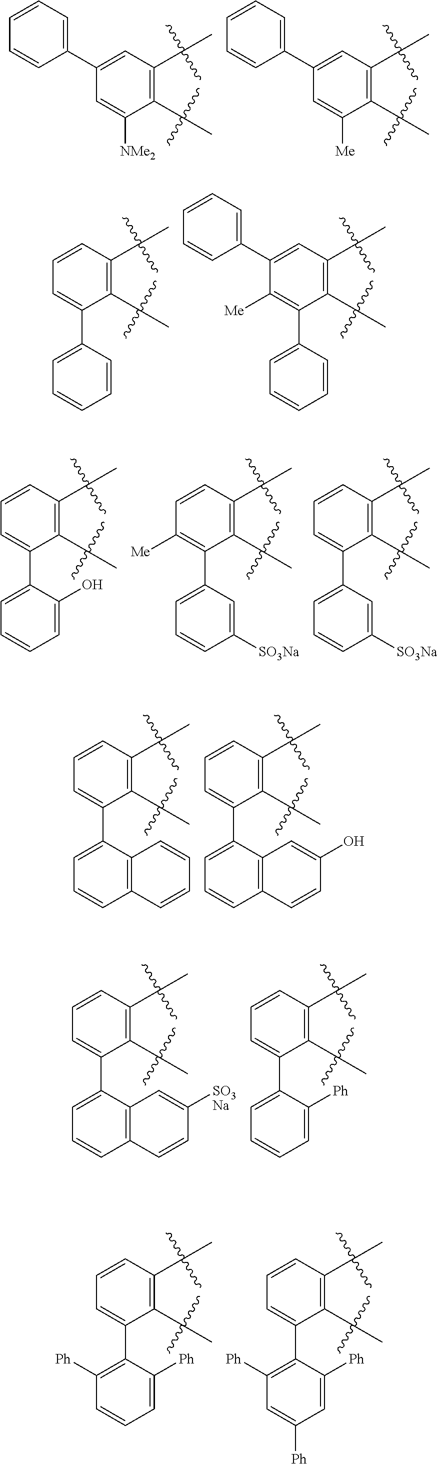

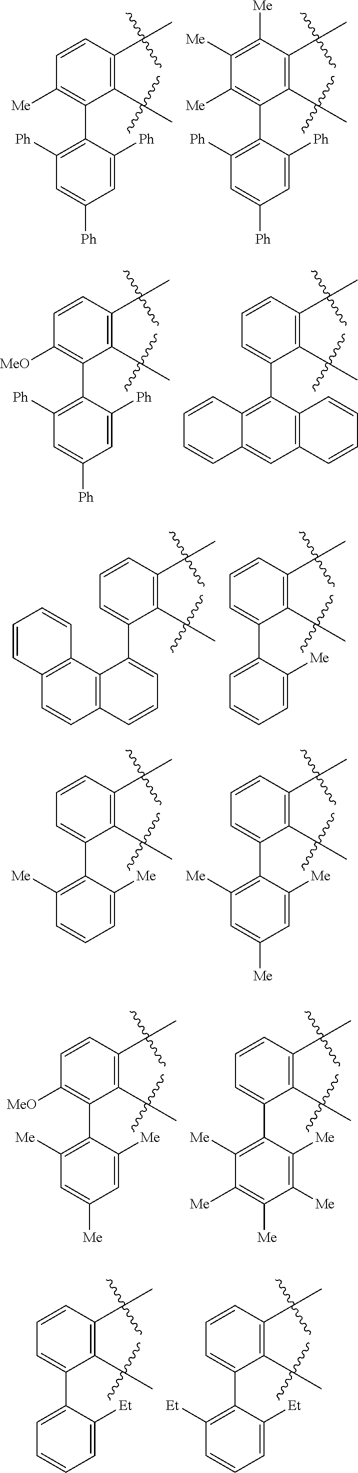

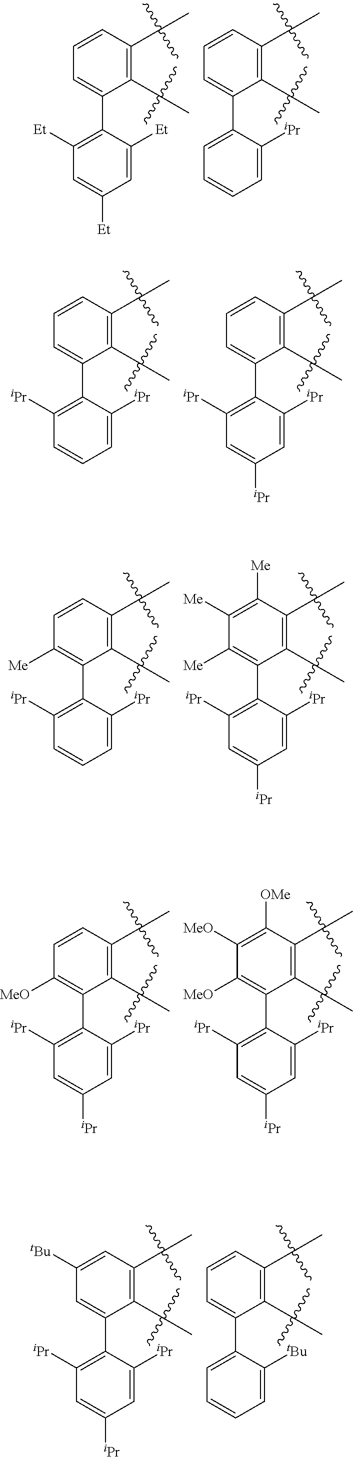

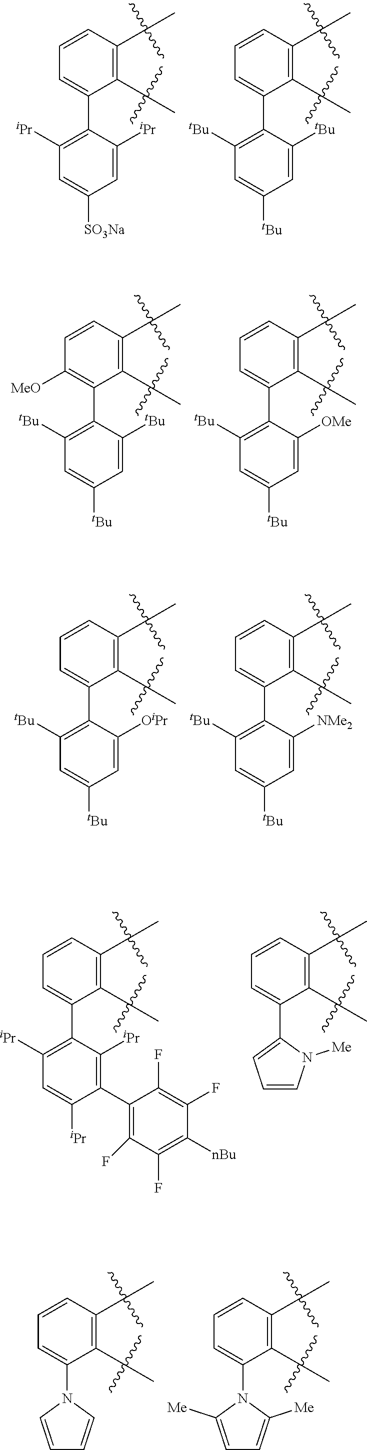

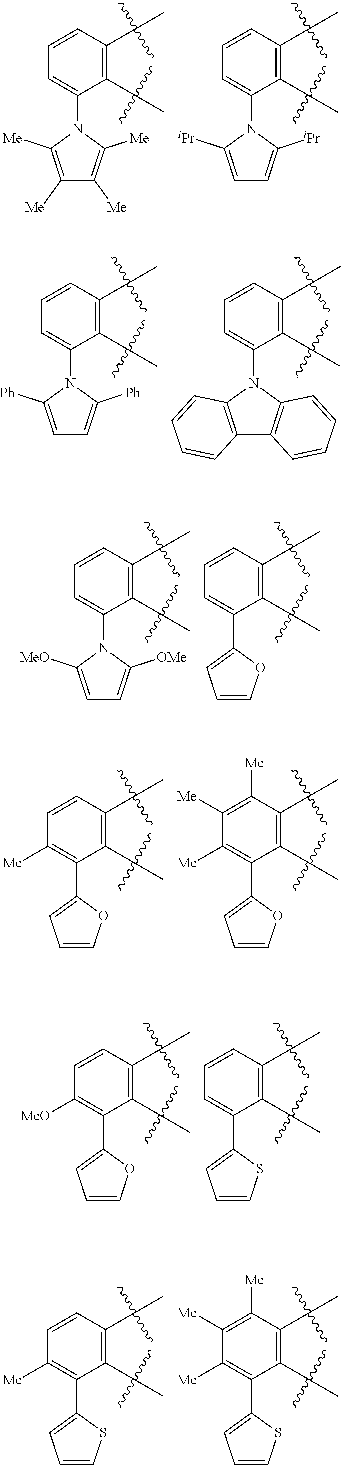

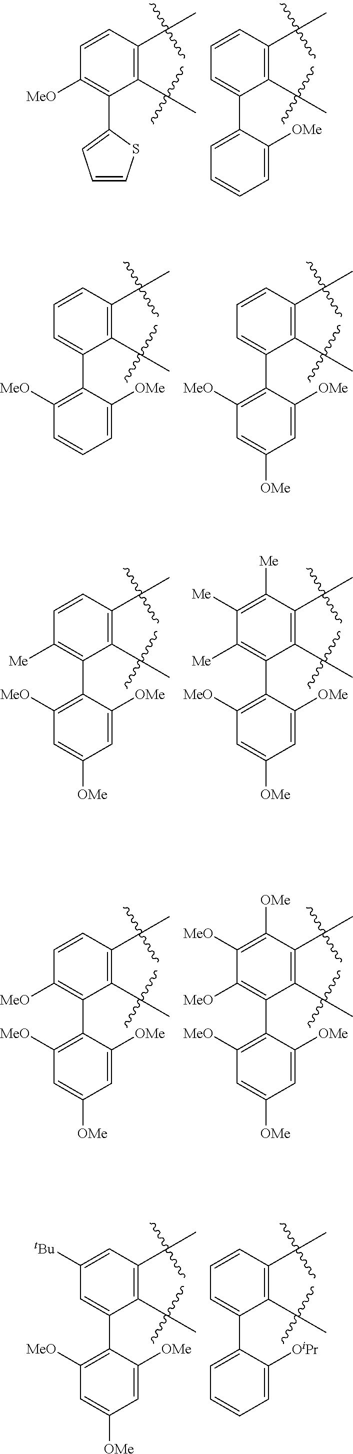

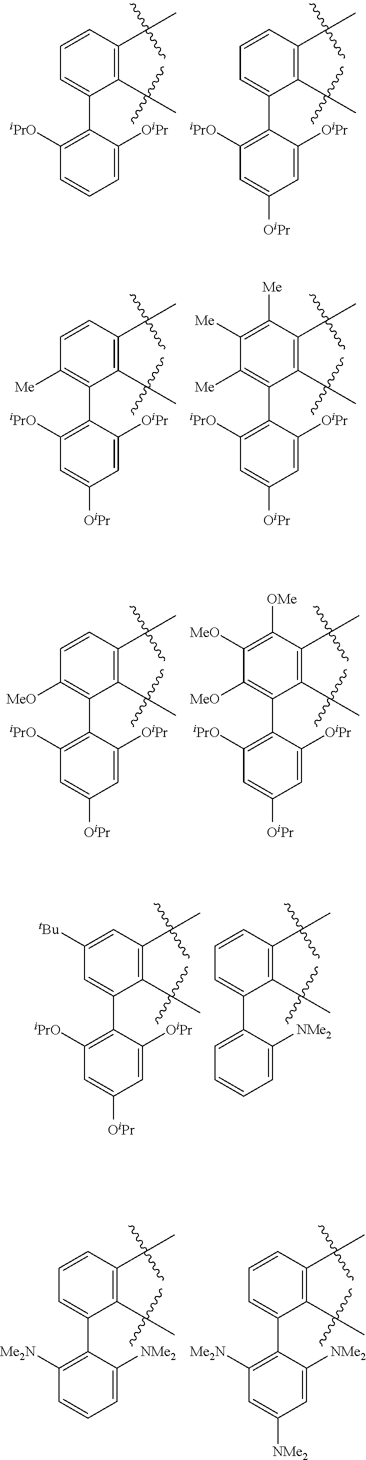

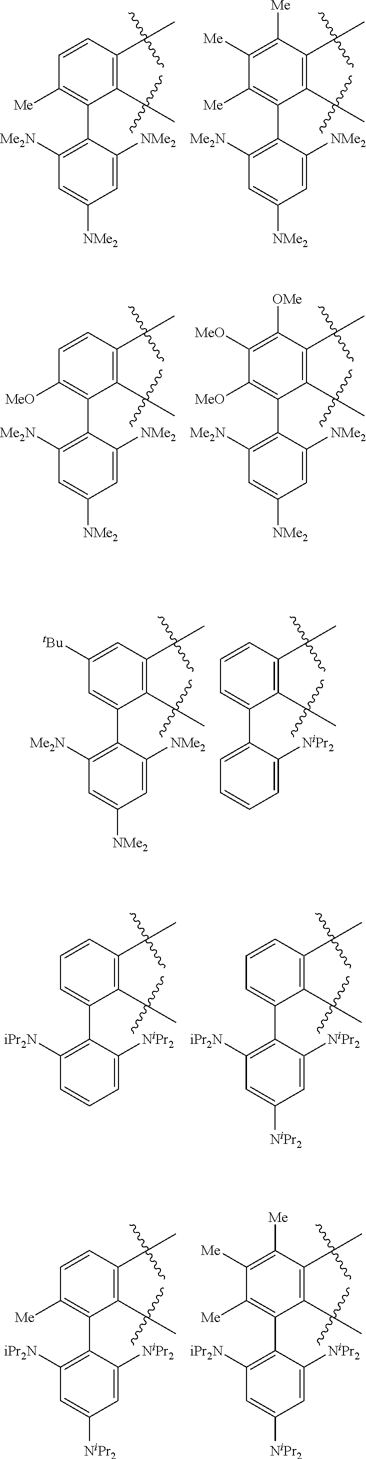

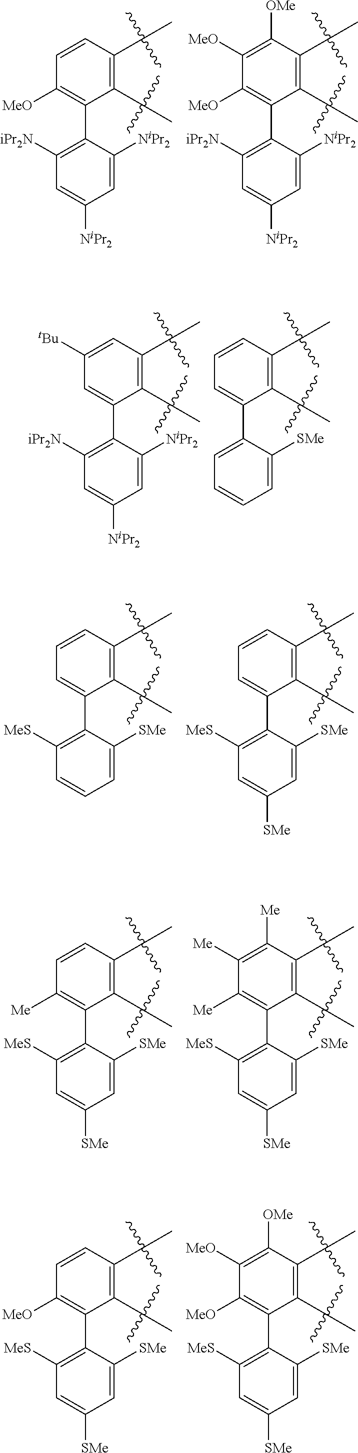

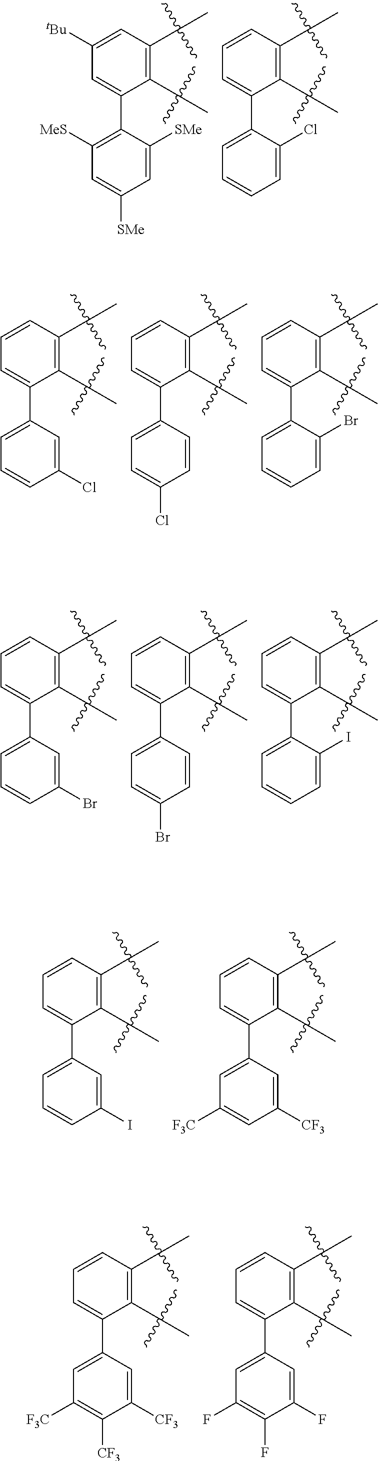

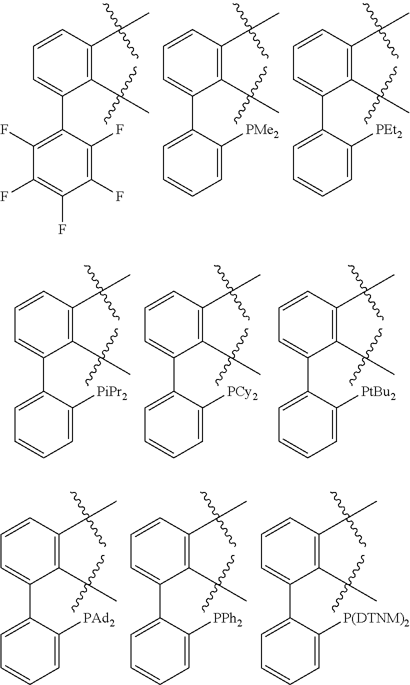

7. The substituted 2,3-dihydrobenzo[d][1,3]oxaphosphole ligand of claim 1, characterised in that R⁴ is selected from:

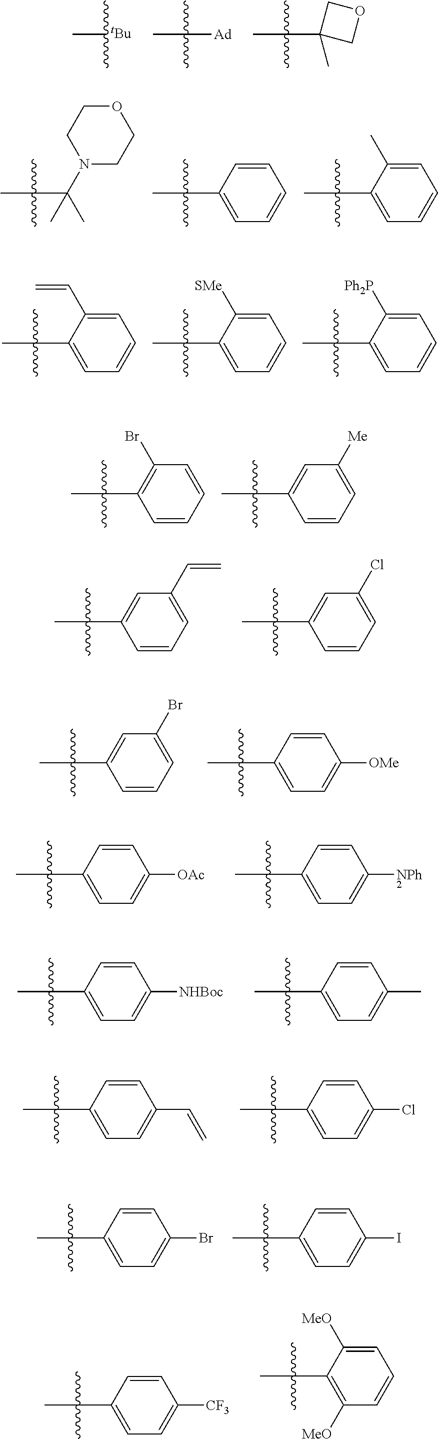

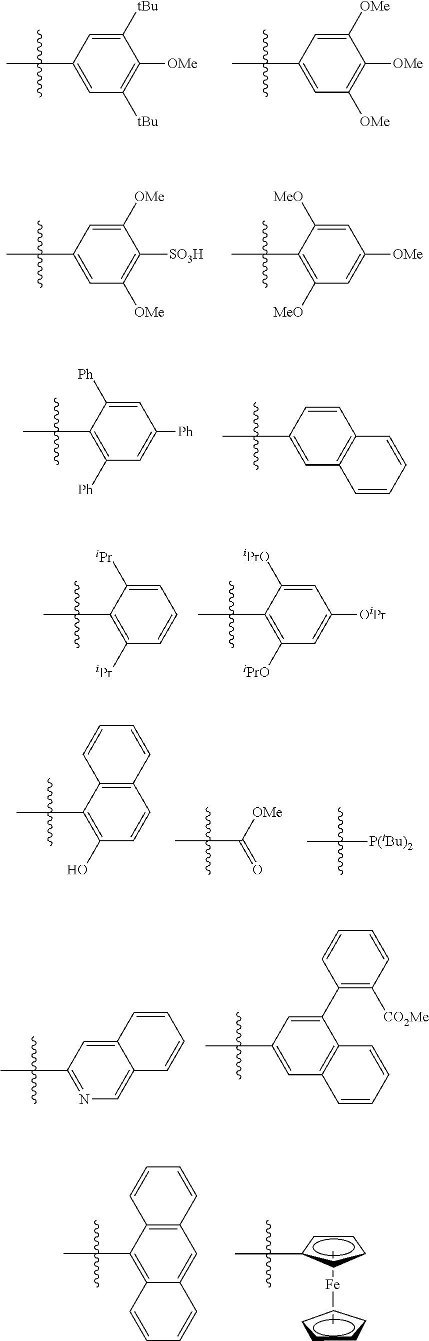

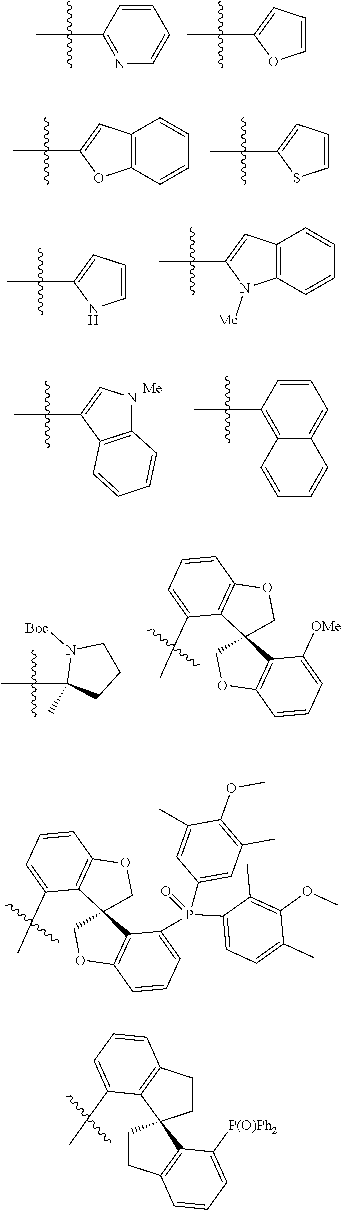

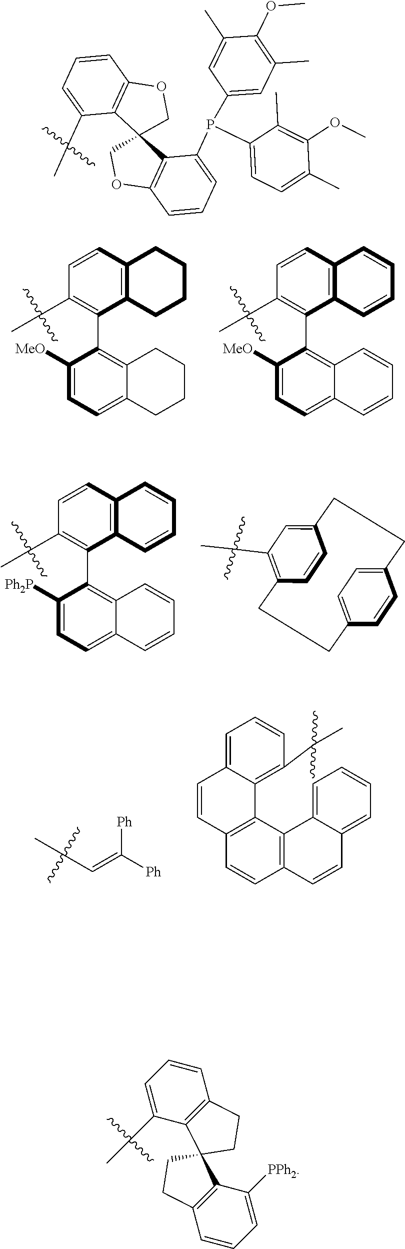

8. The substituted 2,3-dihydrobenzo[d][1,3]oxaphosphole ligand of claim 1, characterised in that each of R³ and R^{3a} are independently selected from:

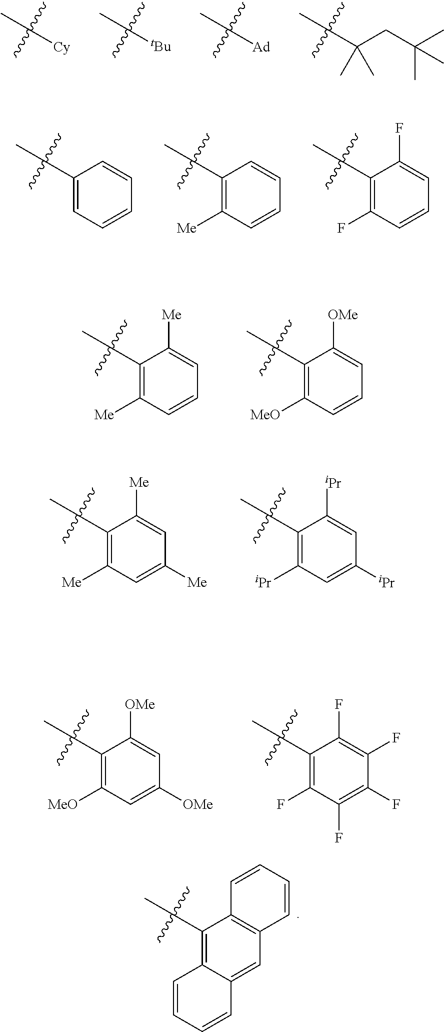

9. The substituted 2,3-dihydrobenzo[d][1,3]oxaphosphole ligand of claim 1, characterised in that Z is selected from:

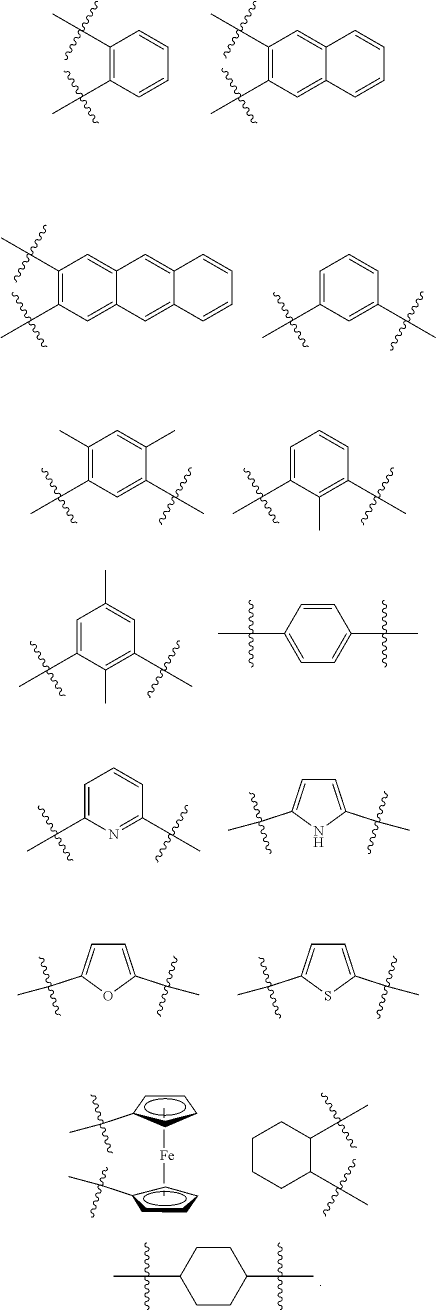

10. The substituted 2,3-dihydrobenzo[d][1,3]oxaphosphole ligand of claim 1, characterised in that the compound of formula (I) is selected from:

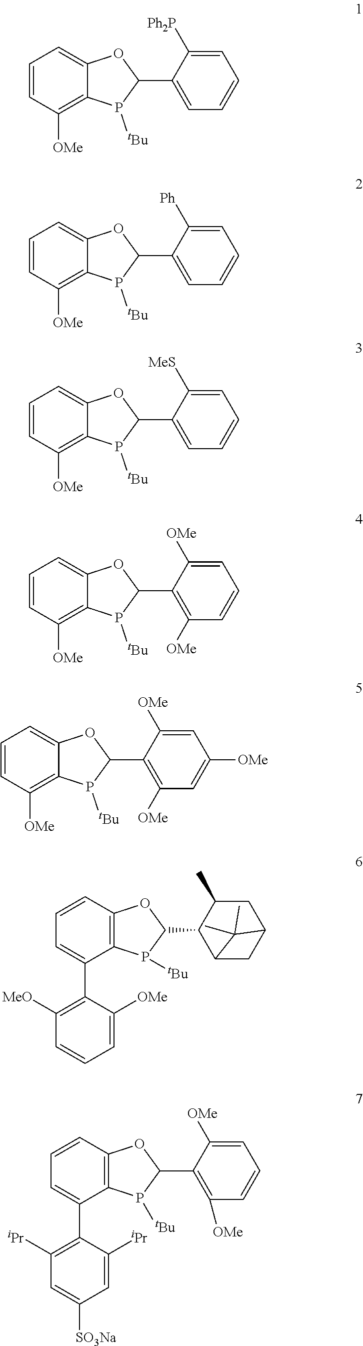

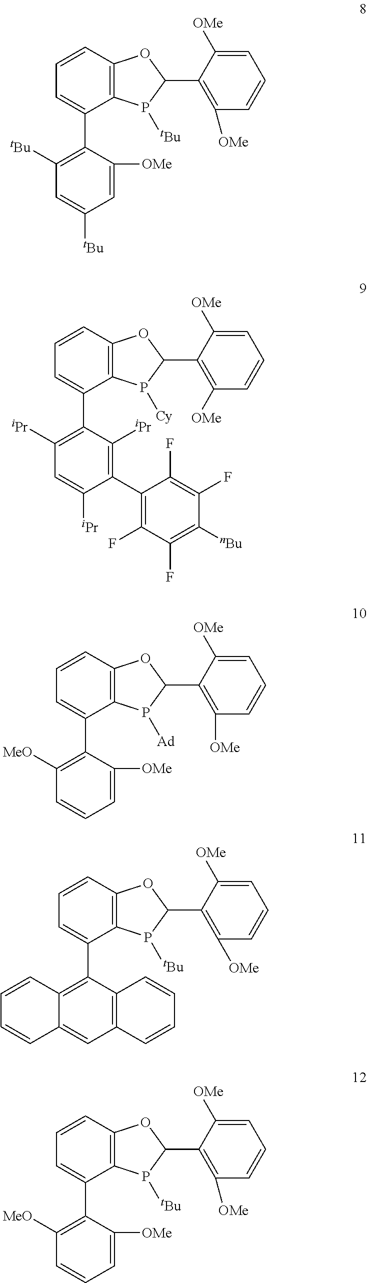

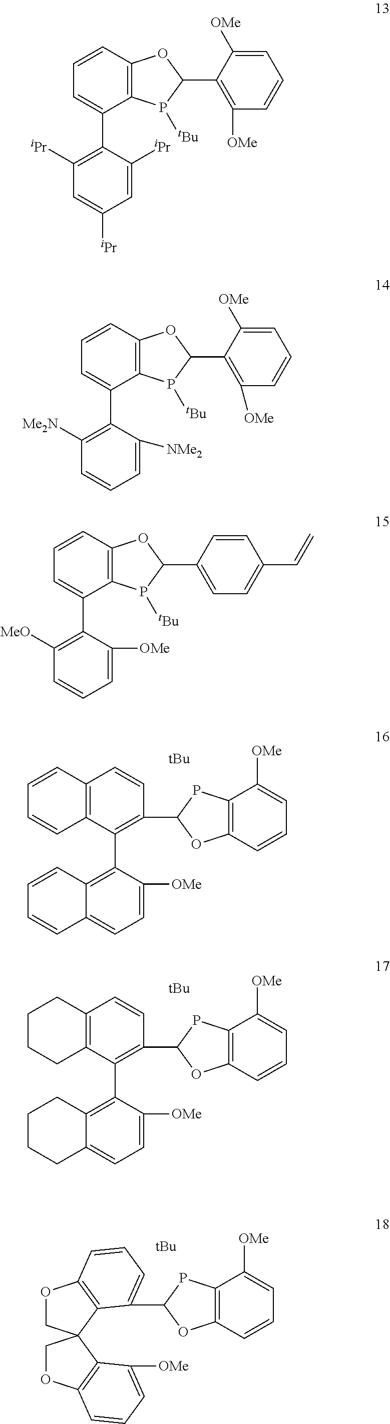

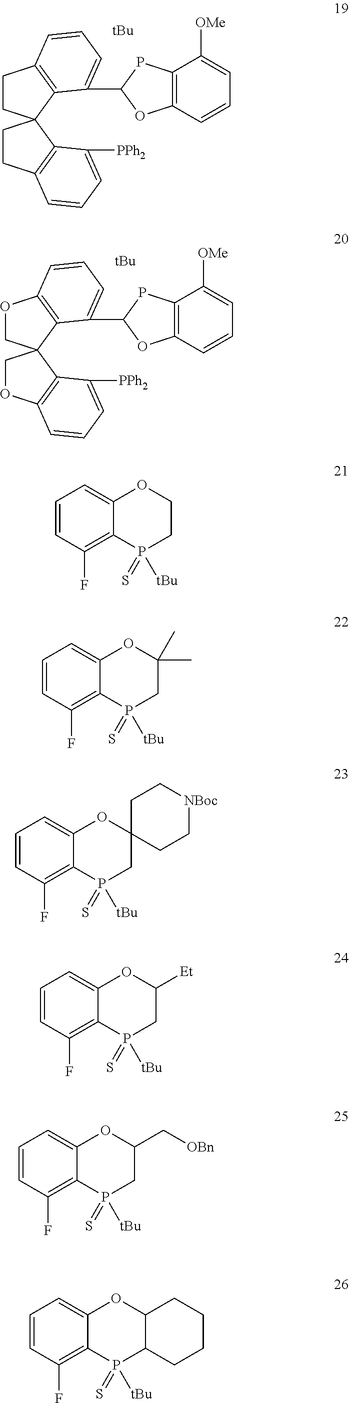

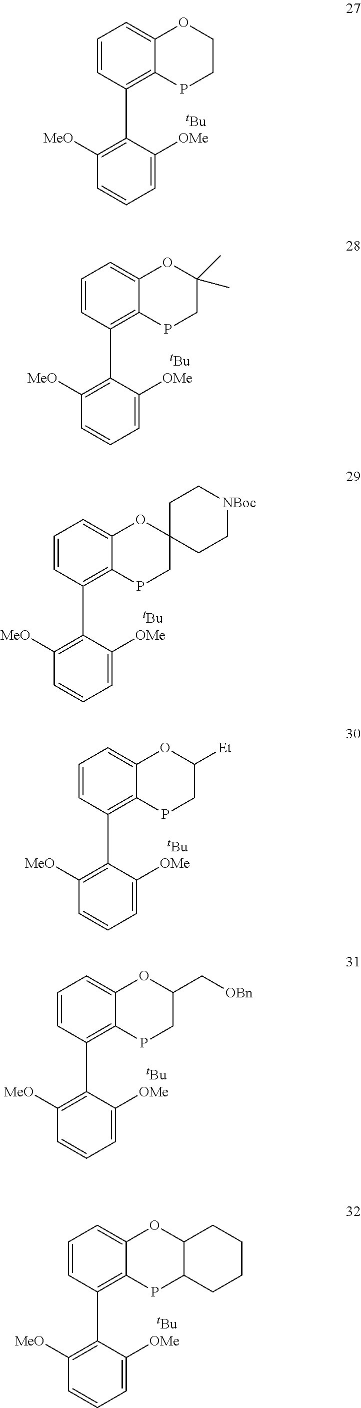

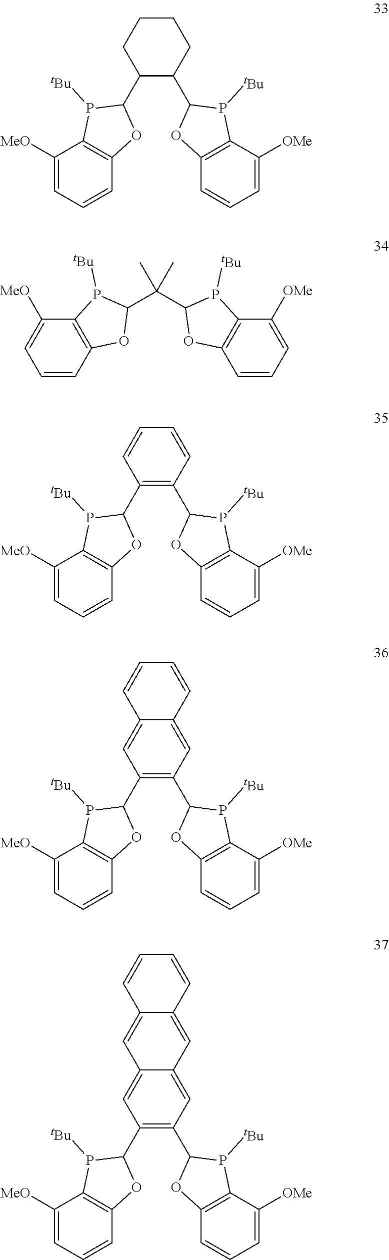 [SMILES: COc1cccc2c1P(C(C)(C)C)C(C1CCCCC1C1Oc3cccc(OC)c3P1C(C)(C)C)O2 ; COc1cccc2c1P(C(C)(C)C)C(C(C)(C)C1Oc3cccc(OC)c3P1C(C)(C)C)O2 ; COc1cccc2c1P(C(C)(C)C)C(c1ccccc1C1Oc3cccc(OC)c3P1C(C)(C)C)O2 ; COc1cccc2c1P(C(C)(C)C)C(c1cc3ccccc3cc1C1Oc3cccc(OC)c3P1C(C)(C)C)O2 ; COc1cccc2c1P(C(C)(C)C)C(c1cc3cc4ccccc4cc3cc1C1Oc3cccc(OC)c3P1C(C)(C)C)O2]

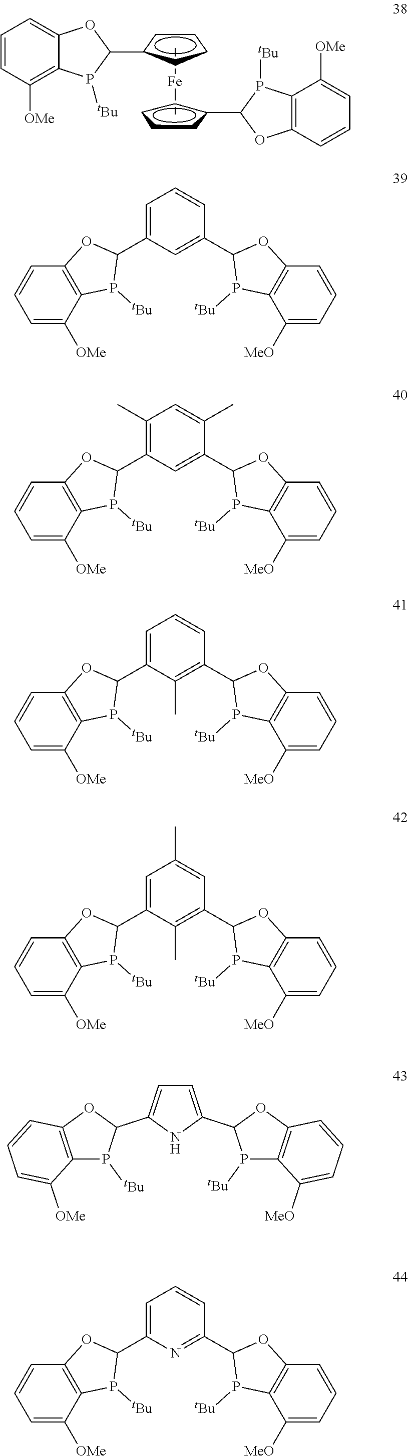

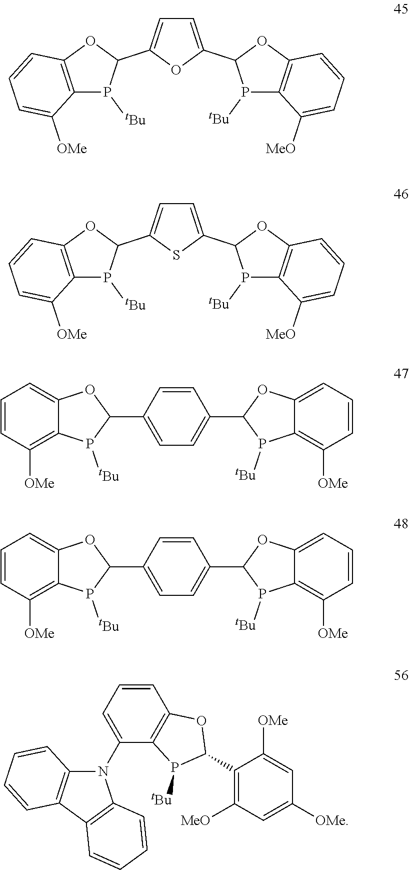 [SMILES: COc1cccc2c1P(C(C)(C)C)C(c1ccc(C3Oc4cccc(OC)c4P3C(C)(C)C)o1)O2 ; COc1cccc2c1P(C(C)(C)C)C(c1ccc(C3Oc4cccc(OC)c4P3C(C)(C)C)s1)O2 ; COc1cccc2c1P(C(C)(C)C)C(c1ccc(C3Oc4cccc(OC)c4P3C(C)(C)C)cc1)O2 ; COc1cccc2c1P(C(C)(C)C)C(c1ccc(C3Oc4cccc(OC)c4P3C(C)(C)C)cc1)O2 ; COc1cc(OC)c([C@H]2Oc3cccc(-n4c5ccccc5c5ccccc54)c3[P@]2C(C)(C)C)c(OC)c1]

11. A diphosphine acetate palladium complex, characterised in that it is represented by the structure of formula (II-B), 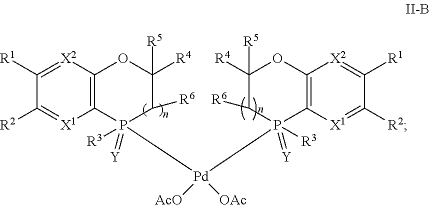
wherein R¹, R², R³, R⁴, R⁵, R⁶, X¹, X³, Y and n have the definition as claimed in claim 1.

12. A coupling reaction method, characterized in that the method uses a biphosphine acetate palladium complex prepared from a biphosphine acetate palladium complex of claim 11 as a catalyst.

13. The method of claim 12, characterized in that the coupling reaction method is C—C coupling reaction, which comprises reacting aryl halides and aryl boronic acid compounds in an organic solvent under alkaline conditions in the presence of the bisphosphonate palladium acetate complex;
or the coupling reaction method is C—N coupling reaction, which comprises reacting aryl halides and aromatic amine compounds in an organic solvent under alkaline conditions in the presence of the bisphosphonate palladium acetate complex;
the solvent is one or more of N,N-dimethylformamide, toluene, benzotrifluoride, carbon tetrachloride, dioxane, hexafluoroisopropanol or ethyl acetate; the base is one or more of potassium tert-butoxide, sodium tert-butoxide, lithium methoxide, cesium carbonate, sodium acetate, sodium hydroxide, potassium hydroxide or sodium hydroxide.

14. The method of claim 13, characterized in that the aryl boronic acid compounds include aryl boronic acid or aryl borate esters; the aryl halides include aryl chlorides and aryl bromides; the aromatic amine compounds include aromatic primary amine or aromatic secondary amine.

15. A preparation method of a substituted 2,3-dihydrobenzo[d][1,3]oxaphosphole ligand comprising:
S11. stirring formula III with a base in an organic solvent to obtain a salt solution of formula III under inert gas protection,
S12. adding the aldehyde of formula IV or epoxide derivative V to the obtained salt solution of formula III, and reacting at 0-50° C. to obtain the 2,3-dihydrobenzo[d][1,3]oxaphosphole derivative of formula I; 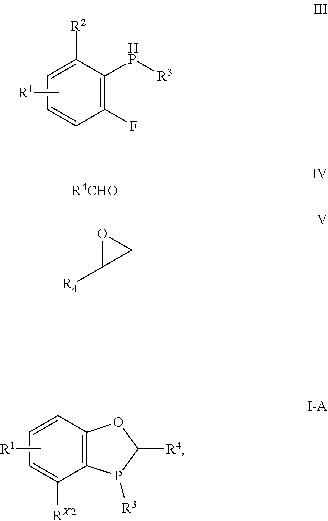
wherein R¹ is H, F or —SO₂NMe₂;
R^{X2} is F, Cl, Br or I;
R³ is aryl or alkyl;
R⁴ is aryl, heteroaryl or alkyl;
the aryl or heteroaryl is unsubstituted or substituted with 1, 2, 3, 4 or 5 substituents selected from halo, —PPh₂, —NH(alkyl), —N(alkyl)₂, C₁₋₆alkyl, C₁₋₆haloalkyl, C₁₋₆alkyloxy and C₁₋₆alkylthio.

16. The method of claim 15, wherein the organic solvent is selected from one or more of 1,4-dioxane, tetrahydrofuran, ether, dichloromethane, 1,2-dichloroethane, toluene, n-hexane, or dimethyl sulfoxide.

17. The method of claim 15, wherein the base is selected from one or more of cesium carbonate, potassium carbonate, sodium carbonate, potassium tert butoxide, sodium tert butoxide, lithium tert butoxide, sodium ethoxide, sodium acetate or sodium hydroxide.

18. The method of claim 15, wherein in the mixed system in step S12, the molar ratio of arylphosphine hydrogen of formula III, aldehyde of formula IV of epoxy compound of formula V, and base is 1:1.5:1.5.

19. Use of the substituted 2,3-dihydrobenzo[d][1,3]oxaphosphole ligand of claim 1 in the coupling reaction of aryl boronic acid compounds and aryl halides; 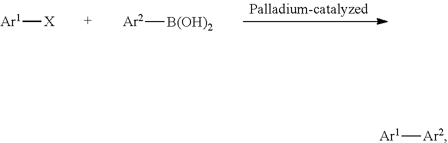
wherein each of Ar¹ and Ar² is independently aryl or heteroaryl.
